SEQUENCE LISTING (36 sequences)

SEQ ID NO: 1 - DNA, 20 bp, synthetic construct
  misc_feature 1..20: note = P5 primer
  source 1..20: mol_type = other DNA
aatgatacggcgaccaccga

SEQ ID NO: 2 - DNA, 24 bp, synthetic construct
  misc_feature 1..24: note = P7 primer
  source 1..24: mol_type = other DNA
caagcagaagacggcatacgagat

SEQ ID NO: 3 - protein, 19 aa, synthetic construct
  REGION 1..19: note = FA-terminal peptide
FAGVAMPGAEDDVVGSGSK

SEQ ID NO: 4 - protein, 20 aa, synthetic construct
  REGION 1..20: note = AFA-terminal peptide
AFAGVAMPGAEDDVVGSGSK

SEQ ID NO: 5 - protein, 19 aa, synthetic construct
  REGION 1..19: note = AA-terminal peptide
AAGVAMPGAEDDVVGSGSK

SEQ ID NO: 6 - protein, 12 aa, synthetic construct
  REGION 1..12: note = Snoop tag
KLGDIEFIKVNK

SEQ ID NO: 7 - protein, 723 aa, Thermomonas hydrothermalis
  REGION 1..723: note = Dipeptidyl peptidase from Thermomonas hydrothermalis (with the signal peptide)
MHKTRLVAALAAALATLAPAAWADEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 8 - protein, 20 aa, synthetic construct
  REGION 1..20: note = Insertion peptide
ESTFEKLRLPSRKVDALDHF

SEQ ID NO: 9 - DNA, 34 bp, synthetic construct
  misc_feature 1..34: note = DBCO-DNA
  source 1..34: mol_type = other DNA
  modified_base 1: mod_base = OTHER; note = 5'-phosphorylated
  modified_base 22: mod_base = OTHER; note = t with the dibenzocyclooctyne group (DBCO)
caagttctcagtaatgcgtagtccgcgacactag

SEQ ID NO: 10 - protein, 749 aa, synthetic construct
  REGION 1..749: note = 10XHis wt Mus musculus TdT (132-510)
MGHHHHHHHHHHSSGHIDDDDKHMSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNANQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 11 - protein, 813 aa, synthetic construct
  REGION 1..813: note = 10XHis wt human Pol theta (1792-2590)
MGHHHHHHHHHHSSGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 12 - protein, 494 aa, Lepisosteus oculatus
  REGION 1..494: note = TdT from Lepisosteus oculatus
MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

SEQ ID NO: 13 - protein, 355 aa, synthetic construct
  REGION 1..355: note = Truncated TdT from Lepisosteus oculatus
TVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

SEQ ID NO: 14 - protein, 494 aa, synthetic construct
  REGION 1..494: note = Mutant TdT from Lepisosteus oculatus
MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNKIFTDAFAILAENAEFNESEGPCLAFRRAASLLKSLPHAISSSKDLEGLPCLGDQTKAVIEDILEYGQCSKVQDVLCDDRYQTIKLFTSVFGVGLKTAEKWYRKGLHSLEEVQADNTIHFTKMQKAGFLYYDDISAAVCKAEADAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIPPWQRNA

SEQ ID NO: 15 - protein, 355 aa, synthetic construct
  REGION 1..355: note = Truncated Mutant TdT from Lepisosteus oculatus
TVSQYACQRRTTMENHNKIFTDAFAILAENAEFNESEGPCLAFRRAASLLKSLPHAISSSKDLEGLPCLGDQTKAVIEDILEYGQCSKVQDVLCDDRYQTIKLFTSVFGVGLKTAEKWYRKGLHSLEEVQADNTIHFTKMQKAGFLYYDDISAAVCKAEADAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIPPWQRNA

SEQ ID NO: 16 - protein, 517 aa, synthetic construct
  REGION 1..517: note = F-binder-Terminal deoxynucleotidyl transferase (TdT) conjugate
GSSHHHHHHGGSSDSPVDLKPKPKVKPKLERPKLYKVMLLNDNSTPGEFVTVVLKAVFRMSEDTGRRVMMTAHRFGSAVVVVSERDIAETKAKEATDLGKEAGFPLMFTTEPEEGAPSGAPSGAPSGAPSGAPSGAPSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 17 - protein, 178 aa, Rattus norvegicus
  REGION 1..178: note = Neutrophil gelatinase-associated lipocalin
QDSTQNLIPAPPLISVPLQPGFWTERFQGRWFVVGLAGNAVQKERQSRFTMYSTIYELQEDNSYNVTSILVRGQGCRYWIRTFVPSSRPGQFTLGNIHSYPQIQSYDVQVADTDYDQFAMVFFQKTSENKQYFKVTLYGRTKGLSDELKERFVSFAKSLGLKDNNIVFSVPTDQCIDN

SEQ ID NO: 18 - protein, 164 aa, Homo sapiens
  REGION 1..164: note = Prostaglandin-H2 D-isomerase
QVSVQPNFQQDKFLGRWFSAGLASNSSWLREKKAALSMAKSVVAPATDGGLNLTSTFLRKNQCETRTMLLQPAGSLGSYSYRSPHFGSTYSVSVVETDYDQYALLYSQGSKGPGEDFRMATLYSRTQTPRAELKEKFTAFSKAQGFTEDTIVFLPQTDKCMTEQ

SEQ ID NO: 19 - protein, 154 aa, Homo sapiens
  REGION 1..154: note = Lipocalin-1 (Tear lipocalin)
ASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYSEGELHGKPVRGVKLVGRDPKNNLEALEDFEKAAGARGLSTESILIPRQSETCSPGSD

SEQ ID NO: 20 - protein, 256 aa, Homo sapiens
  REGION 1..256: note = Carbonic anhydrase II
HWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASF

SEQ ID NO: 21 - protein, 256 aa, Homo sapiens
  REGION 1..256: note = Carbonic anhydrase I
WGYDDKNGPEQWSKLYPIANGNNQSPVDIKTSETKHDTSLKPISVSYNPATAKEIINVGHSFHVNFEDNQDRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 22 - protein, 185 aa, Staphylococcus aureus
  REGION 1..185: note = Peptide deformylase
MLTMKDIIRDGHPTLRQKAAELELPLTKEEKETLIAMREFLVNSQDEEIAKRYGLRSGVGLAAPQINISKRMIAVLIPDDGSGKSYDYMLVNPKIVSHSVQEAYLPTGEGCLSVDDNVAGLVHRHNRITIKAKDIEGNDIQLRLKGYPAIVFQHEIDHLNGVMFYDHIDKDHPLQPHTDAVEVLE

SEQ ID NO: 23 - protein, 698 aa, Pseudoxanthomonas mexicana
  REGION 1..698: note = dipeptide aminopeptidase without signal peptide
GEGMWVPQQLPEIAGPLKKAGLKLSPQQISDLTGDPMGAVVALGGCTASFVSPNGLVVTNHHCAYGAIQLNSTAENNLIKNGFNAPTTADEVSAGPNARVFVLDEITDVTKDAKAAIAAAGDDALARTKALEAFEKKLIADCEAEAGFRCRLYSFSGGNTYRLFKNLEIKDVRLAYAPPGSVGKFGGDIDNWMWPRHTGDFAFYRAYVGKDGKPAAFSKDNVPYQPKHWLKFADQPLGAGDFVMVAGYPGSTNRYALAAEFDNTAQWTYPTIARHYKNQIAMVEAAGKQNADIQVKYAATMAGWNNTSKNYDGQLEGFKRIDAAGQKLREEAAVLGWLKGQGAKGQPALDAHAKLLDLLEQSKATRDRDLTLALFNNTAMLGSATQLYRLSIEREKPNAERESGYQERDLPAIEGGLKQLERRYVAAMDRQLQEYWLNEYIKLPADQRVAAVDAWLGGNDAAAVKRALDRLAGTKLGSTEERLKWFAADRKAFEASNDPAIQYAVAVMPTLLKLEQERKTRAGENLAARPVYLQALADYKKSQGEFVYPDANLSLRITFGNVMGYAPKDGMEYTPFTTLEGVVAKETGQDPFDSPKALLDAVAAKRYGGLEDKRIGSVPVNYLSDLDITGGNSGSPVLDAHGKLVGLAFDGNWESVSSNWVFDPKMTRMIAVDGRYLRWIMQEVYPAPQLLKEMNVGK

SEQ ID NO: 24 - protein, 723 aa, synthetic construct
  REGION 1..723: note = engineered cleavase
MHKTRLVAALAAALATLAPAAWADEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDMGMWPTHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNRTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFAGTLESVASNWVFDPVLTRMISVDQRYMRWIMQEVMPAPQLLEELGVPPRQ

SEQ ID NO: 25 - DNA, 11 bp, synthetic construct
  misc_feature 1..11: note = Test polynucleotide
  source 1..11: mol_type = other DNA
cttcctcttcc

SEQ ID NO: 26 - DNA, 15 bp, synthetic construct
  misc_feature 1..15: note = Test polynucleotide
  source 1..15: mol_type = other DNA
cccctttttgggaaa

SEQ ID NO: 27 - protein, 13 aa, synthetic construct
  REGION 1..13: note = SpyTag
AHIVMVDAYKPTK

SEQ ID NO: 28 - protein, 14 aa, synthetic construct
  REGION 1..14: note = Modified SpyTag
CAHIVMVDAYKPTK

SEQ ID NO: 29 - DNA, 32 bp, synthetic construct
  misc_feature 1..32: note = Illumina sequencing adaptor
  source 1..32: mol_type = other DNA
agatcggaagagcacacgtctgaactccagtc

SEQ ID NO: 30 - protein, 4 aa, synthetic construct
  REGION 1..4: note = Peptide linker
GGGS

SEQ ID NO: 31 - protein, 4 aa, synthetic construct
  REGION 1..4: note = Peptide linker
GSGS

SEQ ID NO: 32 - protein, 11 aa, synthetic construct
  REGION 1..11: note = Peptide linker
GSGTAGGGSGS

SEQ ID NO: 33 - protein, 8 aa, synthetic construct
  REGION 1..8: note = Peptide linker
SGGSGGSG

SEQ ID NO: 34 - protein, 374 aa, Enterobacteria phage T4
  REGION 1..374: note = RNA ligase 1 of Enterobacteria phage T4
MQELFNNLMELCKDSQRKFFYSDDVSASGRTYRIFSYNYASYSDWLLPDALECRGIMFEMDGEKPVRIASRPMEKFFNLNENPFTMNIDLNDVDYILTKEDGSLVSTYLDGDEILFKSKGSIKSEQALMANGILMNINHHRLRDRLKELAEDGFTANFEFVAPTNRIVLAYQEMKIILLNVRENETGEYISYDDIYKDATLRPYLVERYEIDSPKWIEEAKNAENIEGYVAVMKDGSHFKIKSDWYVSLHSTKSSLDNPEKLFKTIIDGASDDLKAMYADDEYSYRKIEAFETTYLKYLDRALFLVLDCHNKHCGKDRKTYAMEAQGVAKGAGMDHLFGIIMSLYQGYDSQEKVMCEIEQNFLKNYKKFIPEGY

SEQ ID NO: 35 - protein, 7 aa, synthetic construct
  REGION 1..7: note = Test peptide
FARDCSN

SEQ ID NO: 36 - protein, 27 aa, synthetic construct
  REGION 1..27: note = Test peptide
  SITE 6: note = linked to azide
CGSGSKSGGSGGSGAHIVMVDAYKPTK

(19) United States
(12) Patent Application Publication
(10) Pub. No.: US 2024/0053350 A1
(43) Pub. Date: Feb. 15, 2024

(54) HIGH THROUGHPUT PEPTIDE IDENTIFICATION USING CONJUGATED BINDERS AND KINETIC ENCODING

(71) Applicant: Encodia, Inc., San Diego, CA (US)

(72) Inventor: Maziar S. ARDEJANI, San Diego, CA (US)

(21) Appl. No.: 18/466,543

(22) Filed: Sep. 13, 2023

Related U.S. Application Data
(63) Continuation of application No. PCT/US2023/065816, filed on Apr. 14, 2023.
(60) Provisional application No. 63/331,702, filed on Apr. 15, 2022.

(51) Int. Cl. G01N 33/68; G01N 33/53; G01N 33/543

(52) U.S. Cl. CPC G01N 33/6803; G01N 33/5308; G01N 33/54306

(57) ABSTRACT

The present disclosure relates to methods and kits for high-throughput, highly parallel peptide identification employing immobilization of peptides associated with nucleic acid recording tags on a solid support, followed by contacting of immobilized peptides with peptide-binding agents conjugated to a writer enzyme, which is capable of catalyzing covalent addition of a nucleic acid moiety (e.g., nucleotides) onto the nucleic acid recording tags located in proximity. As a result, structural information of immobilized peptides, as well as kinetic information of binder-peptide interactions, can be encoded into nucleotide sequences associated with the peptides. Finally, identities of immobilized peptides can be decoded using sequences of the associated recording tags.

### CROSS-REFERENCE TO RELATED APPLICATIONS

This application is a continuation of International Patent Application No. PCT/US2023/065816, filed Apr. 14, 2023, entitled “HIGH THROUGHPUT PEPTIDE IDENTIFICATION USING CONJUGATED BINDERS AND KINETIC ENCODING,” which claims priority to U.S. Provisional Application No. 63/331,702, filed Apr. 15, 2022, which are herein incorporated by reference in their entireties for all purposes.

### REFERENCE TO AN ELECTRONIC SEQUENCE LISTING

The contents of the electronic sequence listing (776532004301SEQLIST.xml; Size: 55,112 bytes; and Date of Creation: Sep. 11, 2023) is herein incorporated by reference in its entirety.

### TECHNICAL FIELD

This disclosure generally relates to biotechnology, and in particular to highly parallel identification of peptide(s), which utilizes conjugates of peptide-binding agents and a writer enzyme, as well as nucleic acid encoding of molecular recognition events. The disclosure finds utility in a variety of methods and related kits for high-throughput peptide identification with applications in various fields, e.g., biology and medicine.

### BACKGROUND

High-throughput identification of proteins remains challenging for several reasons. The use of affinity-based assays is often difficult due to several challenges. One significant challenge is multiplexing the readout of a collection of affinity agents to a collection of cognate peptides; another challenge is minimizing cross-reactivity between the affinity agents and off-target peptides; and a third challenge is developing an efficient high-throughput read-out platform.

Recently, methods have been disclosed that utilize binding agents for high-throughput peptide identification and sequencing, for example, U.S. Pat. Nos. 9,435,810 B2, 10,473,654 B1, 9,625,469 B2, 10,006,917 B2, and the following published patent applications: WO2010065531A1, US 20190145982 A1, US 20200217853 A1, US 20200348308 A1, US 20200400677 A1, US 20200217853 A1, and US 20180299460 A1. Some of these methods utilize N-terminal amino acid (NTAA) recognition by binding agents as a critical step in a peptide identification and sequencing assay. A number of methods to evolve specific NTAA binders from different scaffolds for recognizing a particular terminal amino acid have also been proposed, including directed evolution approaches to derive amino acyl tRNA synthetases, N-recognins such as ClpS and ClpS2, anticalins, and aminopeptidases, which are disclosed, for example, in U.S. Pat. No. 9,435,810 B2 and US patent publication 2019/0145982 A1. However, identifying binding agents that afford amino acid specificity with sufficiently strong affinity has proven challenging. Binding affinity and/or specificity towards an N-terminal amino acid residue (P1) can vary depending on neighboring amino acid residues of the peptide to be analyzed, e.g., the penultimate amino acid residue (P2) and the antepenultimate amino acid residue (P3). In addition, engineered NTAA binders may exhibit less selective binding, where the binder may bind with similar affinity to two or more different amino acid residues. Accordingly, there remains a need for improved techniques relating to identification of peptide(s) in a sample. The present disclosure addresses this and the related needs.

### BRIEF SUMMARY

The present disclosure describes novel and improved approaches for performing highly-parallel identification of peptides by utilizing peptide-binding agents conjugated to a writer enzyme, so that structural information of peptides is encoded as nucleotide sequences associated with the peptides. These and other aspects of the disclosure will be apparent upon reference to the following detailed description. Various references are set forth herein which describe in more detail certain background information, procedures, compounds and/or compositions, and are each hereby incorporated by reference in their entireties.

The summary is not intended to be used to limit the scope of the claimed subject matter. Other features, details, utilities, and advantages of the claimed subject matter will be apparent from the detailed description including those aspects disclosed in the accompanying drawings and in the appended claims.

Several variants of the ProteoCode™ assay that allow for high-throughput peptide characterization and identification have been disclosed in the following published patent applications: US 2019/0145982 A1, US 2020/0348308 A1, US 2020/0348307 A1 and US 2021/0208150 A1. During an exemplary assay, an immobilized peptide associated with a nucleic acid recording tag is contacted with binding agents capable of binding to the peptide, wherein each binding agent comprises a nucleic acid coding tag with identifying information regarding the binding agent. During each binding cycle, the coding tag and the recording tag are located in a sufficient proximity for interaction, and the information regarding the binding agent that bound to the peptide at this cycle is transferred between the coding tag to the recording tag, e.g., from the coding tag to the recording tag, thus generating an extended recording tag.

After two or more successive binding cycles, a nucleic acid encoded library representative of the binding history of the macromolecule is generated and encoded in the extended recording tag. Following the analysis of the extended recording tag (usually by a nucleic acid sequencing method), information about the binding agents bound to the peptide at each cycle can be decoded, providing information regarding components of the peptide to which the binding agents were bound. Thus, the ProteoCode™ assay represents an unconventional way of characterizing, identifying or quantifying the peptide's components, and is suitable for highly-parallel, high-throughput peptide characterization, such as peptide identification and/or de novo sequencing.

Provided herein is an alternative workflow and architecture for high-throughput peptide identification assay that utilizes similar immobilization of peptide(s) associated with a recording tag on a solid support and in addition employs peptide-binding agents conjugated to a writer enzyme, which is capable of catalyzing covalent addition of a nucleic acid moiety to a terminus of the recording tag. As a result, structural information of immobilized peptide(s) is encoded as nucleotide sequences. Then, identities of immobilized peptide(s) can be decoded from sequences of corresponding associated recording tags by calculating probabilities of occurrence of specific types of amino acid residues in corresponding places in amino acid sequences of the peptide(s).

Potential advantages of this approach compared to the previously described Proteocode™ assay include: a) removing interference of coding tag-recording tag (such as DNA-DNA) interaction during binder-peptide interaction, which may reduce the background signal; b) removing requirement for amino acid-selective binders that specifically bind to a particular type of NTAA residues (such as Ala-specific binder or Glu-specific binder). Binders in the described approach may be less selective and recognize, for example, functional classes of NTAA residues, such as negatively charged residues, positively charged residues, small hydrophobic residues, aromatic residues, and so on, or recognize other NTAA residue types. This is the case because several binders can be used simultaneously for decoding on a single NTAA residue, and in addition, kinetic information of binder-NTAA interaction can also be encoded.

In some embodiments, provided herein is a method for analyzing a peptide, wherein the peptide and/or an associated nucleic acid recording tag are joined to a support. In some embodiments, the peptide is joined (e.g., covalently or noncovalently, and/or directly or indirectly via a linker) to the support, and the associated nucleic acid recording tag is joined (e.g., covalently or noncovalently, and/or directly or indirectly via a linker) to the peptide. For example, the associated nucleic acid recording tag can be joined to the support via the peptide. In some embodiments, the associated nucleic acid recording tag is joined (e.g., covalently or noncovalently, and/or directly or indirectly via a linker) to the support, and the peptide is joined (e.g., covalently or noncovalently, and/or directly or indirectly via a linker) to the associated nucleic acid recording tag. For example, the peptide can be joined to the support via the associated nucleic acid recording tag. In some embodiments, the peptide and the associated nucleic acid recording tag is each joined (e.g., covalently or noncovalently, and/or directly or indirectly via a linker) to the support independent of one another, and the peptide and the associated nucleic acid recording tag are in vicinity (e.g., the peptide and the nucleic acid recording tag can be “associated” with each other due to their co-localization on the support).

One embodiment of this disclosure provides a method for analyzing a peptide, wherein the peptide and/or an associated nucleic acid recording tag are joined to a support, the method comprising: a) contacting the peptide with a first composition comprising a first conjugate and a first nucleic acid moiety, wherein the first conjugate comprises a first binding agent that binds to the peptide, wherein the first binding agent is conjugated to a first writer enzyme that catalyzes covalent addition of the first nucleic acid moiety to a terminus of the nucleic acid recording tag to generate an extended nucleic acid recording tag joined to the support; b) contacting the peptide with a second composition comprising a second conjugate and a second nucleic acid moiety, wherein the second conjugate comprises a second binding agent that binds to the peptide, wherein the second binding agent is conjugated to a second writer enzyme capable of catalyzing covalent addition of the second nucleic acid moiety to a terminus of the extended nucleic acid recording tag to generate a further extended nucleic acid recording tag joined to the support; and c) analyzing the further extended nucleic acid recording tag to obtain information regarding binding kinetics and/or selectivity of the first binding agent binding to the peptide and information regarding binding kinetics and/or selectivity of the second binding agent binding to the peptide, thereby analyzing the peptide.

In some embodiments, the peptide is contacted with the first composition and with the second composition sequentially, e.g., the peptide can be contacted with the first composition (optionally followed by removing excess molecules of the first conjugate and/or the first nucleic acid moiety) and then with the second composition, or vice versa.

In some embodiments, the peptide is contacted with the first composition and with the second composition simultaneously. The first and second compositions can be contacted with the peptide as separate compositions or pre-mixed, followed by contacting the mixture with the peptide.

In some embodiments, the method comprises: a) contacting the peptide with a first composition comprising a first conjugate and a first nucleic acid moiety, wherein the first conjugate comprises a first binding agent that binds to a terminal amino acid (TAA) or a modified TAA of the peptide, wherein the first binding agent is conjugated to a first writer enzyme that catalyzes covalent addition of the first nucleic acid moiety to a terminus of the nucleic acid recording tag to generate an extended nucleic acid recording tag joined to the support; b) contacting the peptide with a second composition comprising a second conjugate and a second nucleic acid moiety, wherein the second conjugate comprises a second binding agent that binds to the terminal amino acid (TAA) or the modified TAA of the peptide, wherein the second binding agent is conjugated to a second writer enzyme that catalyzes covalent addition of the second nucleic acid moiety to a terminus of the extended nucleic acid recording tag to generate a further extended nucleic acid recording tag joined to the support, wherein the further extended nucleic acid recording tag is identified to obtain information regarding binding kinetics and/or selectivity of the first binding agent binding to the TAA or the modified TAA and information regarding binding kinetics and/or selectivity of the second binding agent binding to the TAA or the modified TAA, thereby identifying the TAA or the modified TAA of the peptide.

In some embodiments, the method comprises c) cleaving the peptide to generate a cleaved peptide, thereby removing the TAA or the modified TAA to expose a new TAA, and, optionally, modifying the new TAA to yield a newly modified TAA. In some embodiments, the method comprises contacting the peptide with a third composition comprising a third conjugate and a third nucleic acid moiety, wherein the third conjugate comprises a third binding agent that binds to the terminal amino acid (TAA) or the modified TAA of the peptide, wherein the third binding agent is conjugated to a third writer enzyme that catalyzes covalent addition of the third nucleic acid moiety to a terminus of the further extended nucleic acid recording tag to generate an even further extended nucleic acid recording tag joined to the support, wherein the even further extended nucleic acid recording tag is identified to obtain information regarding binding kinetics and/or selectivity of the first binding agent binding to the TAA or the modified TAA, information regarding binding kinetics and/or selectivity of the second binding agent binding to the TAA or the modified TAA, and information regarding binding kinetics and/or selectivity of the third binding agent binding to the TAA or the modified TAA, thereby identifying the TAA or the modified TAA of the peptide.

Provided herein is also a method for analyzing a peptide, wherein the peptide and/or an associated nucleic acid recording tag are joined to a support, the method comprising the steps of a) contacting the peptide with a mixture of compositions comprising a first composition and a second composition, wherein (i) the first composition comprises a first conjugate and a first nucleic acid moiety; the first conjugate comprises a first binding agent that binds to the peptide; the first binding agent is conjugated to a first writer enzyme that catalyzes covalent addition of the first nucleic acid moiety to a terminus of the nucleic acid recording tag; and the first nucleic acid moiety is tethered to and controllably cleavable from the first writer enzyme; (ii) the second composition comprises a second conjugate and a second nucleic acid moiety; the second conjugate comprises a second binding agent that binds to the peptide; the second binding agent is conjugated to a second writer enzyme that catalyzes covalent addition of the second nucleic acid moiety to the terminus of the nucleic acid recording tag; and the second nucleic acid moiety is tethered to and controllably cleavable from the second writer enzyme, thereby generating an extended nucleic acid recording tag joined to the support, wherein the extended nucleic acid recording tag comprises covalent addition of the first and/or second nucleic acid moiety; b) cleaving the first nucleic acid moiety from the first writer enzyme and/or cleaving the second nucleic acid moiety from the second writer enzyme, thereby releasing the first and/or second writer enzyme from the extended nucleic acid recording tag; c) optionally, repeating steps (a) and (b) one or more times to generate a further extended nucleic acid recording tag joined to the solid support; and d) analyzing the extended nucleic acid recording tag or the further extended nucleic acid recording tag and obtaining information regarding binding kinetics and/or selectivity of the binding agents bound to the peptide, thereby analyzing the peptide.

Provided herein is also a method for identifying a component of a peptide, the method comprising the steps of: (a) providing the peptide and/or an associated nucleic acid recording tag joined to a solid support; (b) contacting the peptide with a first composition comprising a first conjugate and a first nucleic acid moiety, wherein the first conjugate comprises a first binding agent capable of binding to the peptide, wherein the first binding agent is conjugated to a writer enzyme capable of catalyzing covalent addition of the first nucleic acid moiety to a terminus of the nucleic acid recording tag; (c) following the binding of the first conjugate to the peptide, allowing the writer enzyme of the first conjugate to catalyze covalent addition of the first nucleic acid moiety to the terminus of the nucleic acid recording tag to generate an extended nucleic acid recording tag joined to the solid support; (d) contacting the peptide with a second composition comprising a second conjugate and a second nucleic acid moiety, wherein the second conjugate comprises a second binding agent capable of binding to the peptide, wherein the second binding agent is conjugated to a writer enzyme capable of catalyzing covalent addition of the second nucleic acid moiety onto a terminus of the extended nucleic acid recording tag; (e) following the binding of the second conjugate to the peptide, allowing the writer enzyme of the second conjugate to catalyze covalent addition of the second nucleic acid moiety to the terminus of the extended nucleic acid recording tag to generate a further extended nucleic acid recording tag joined to the solid support; (f) optionally, repeating steps (d)-(e) one or more times by replacing the second composition with a third or higher order composition comprising a third or higher order conjugate and a third or higher order nucleic acid moiety, wherein the third or higher order conjugate comprises a third or higher order binding agent capable of binding to the peptide fused via a flexible linker to a writer enzyme capable of catalyzing covalent addition of the third or higher order nucleic acid moiety onto a terminus of the extended or further extended nucleic acid recording tag; and by allowing the writer enzyme of the third or higher order conjugate to catalyze covalent addition of the third or higher order nucleic acid moiety to the terminus of nucleic acid recording tag extended after step (e) or after previous addition(s) to generate a further extended nucleic acid recording tag joined to the solid support; and (g) analyzing the further extended nucleic acid recording tag and obtaining information regarding binding kinetics or selectivity of the first binding agent binding to the peptide and information regarding binding kinetics or selectivity of the second binding agent binding to the peptide, thereby identifying a component of the peptide.

Provided herein is also a method for identifying a component of a peptide, the method comprising the steps of: (a) providing the peptide and/or an associated nucleic acid recording tag joined to a solid support; (b) contacting the peptide with a mixture comprising a first composition, a second composition, and, optionally, a third or higher order composition, wherein (i) the first composition comprises a first conjugate and a first nucleic acid moiety; the first conjugate comprises a first binding agent capable of binding to the peptide; the first binding agent is conjugated to a writer enzyme capable of catalyzing covalent addition of the first nucleic acid moiety to a terminus of the nucleic acid recording tag; and the first nucleic acid moiety is covalently tethered to the writer enzyme via a linker comprising a selectively cleavable linkage; (ii) the second composition comprises a second conjugate and a second nucleic acid moiety; the second conjugate comprises a second binding agent capable of binding to the peptide; the second binding agent is conjugated to a writer enzyme capable of catalyzing covalent addition of the second nucleic acid moiety to a terminus of the nucleic acid recording tag; and the second nucleic acid moiety is covalently tethered to the writer enzyme via a linker comprising a selectively cleavable linkage; (iii) the third or higher order composition comprises a third or higher order conjugate and a third or higher order nucleic acid moiety; the third or higher order conjugate comprises a third or higher order binding agent capable of binding to the peptide; the third or higher order binding agent is conjugated to a writer enzyme capable of catalyzing covalent addition of the third or higher order nucleic acid moiety to a terminus of the nucleic acid recording tag; and the third or higher order nucleic acid moiety is covalently tethered to the writer enzyme via a linker comprising a selectively cleavable linkage; (c) following binding of the first conjugate, the second conjugate or the third or higher order conjugate to the peptide, allowing the writer enzyme of the conjugate bound to the peptide to catalyze covalent addition of the nucleic acid moiety covalently tethered to the writer enzyme to the terminus of the nucleic acid recording tag to generate an extended nucleic acid recording tag joined to the solid support; (d) cleaving the selectively cleavable linkage and releasing the writer enzyme from the nucleic acid moiety on the terminus of the extended nucleic acid recording tag; (e) optionally, repeating steps (b)-(d) one or more times to generate a further extended nucleic acid recording tag joined to the solid support; and (f) analyzing the extended nucleic acid recording tag or the further extended nucleic acid recording tag and obtaining information regarding binding kinetics or selectivity of the binding agent(s) bound to the peptide, thereby identifying a component of the peptide.

In some embodiments, provided herein is a conjugate which comprises a binding agent conjugated via a linker to a writer enzyme, wherein said conjugate is configured to bind to a peptide comprising an associated nucleic acid recording tag joined to a solid support, said binding agent is configured to bind to said peptide, and said writer enzyme is configured to catalyze covalent addition of a nucleotide moiety onto a terminus of said nucleic acid recording tag. In some embodiments, provided herein is a conjugate which comprises a binding agent conjugated via a first linker to a writer enzyme, wherein said conjugate is configured to bind to a peptide, wherein the peptide and an associated nucleic acid recording tag are joined to a support, said binding agent is configured to bind to said peptide, and said writer enzyme is configured to catalyze covalent addition of a nucleic acid moiety onto a terminus of said nucleic acid recording tag.

In some embodiments, provided herein is a composition comprising two or more conjugates (e.g., conjugates of any one or more embodiments of the present disclosure), wherein each conjugate comprises a binding agent conjugated via a first linker to a writer enzyme, wherein each binding agent is configured to bind to a peptide, wherein the peptide and an associated nucleic acid recording tag are joined to a support, and each writer enzyme is configured to catalyze covalent addition of a nucleic acid moiety onto a terminus of each nucleic acid recording tag.

Yet another embodiment of this disclosure provides a kit for analyzing or identifying a peptide or a component (e.g., an amino acid sequence of one or more residues) thereof, which kit comprises a conjugate or a composition as described above, and an instruction for using the conjugate or the composition for analyzing or identifying the peptide or component thereof.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIGS. 1A-1B show two exemplary designs of a conjugate that comprises a binding agent (binder) capable of binding to an immobilized peptide (e.g., at an NTAA residue of the peptide) conjugated to a writer enzyme (e.g., a T4 DNA ligase, a T4 RNA ligase, or a template-independent polymerase, such as TdT) capable of catalyzing covalent addition of a nucleic acid moiety onto a nucleic acid recording tag (RT) associated with the peptide. Several diverse binder-writer conjugates that recognize different physicochemical classes ofNTAA residues (modified NTAA residues or unmodified NTAA residues) with different affinities towards particular NTAA residues are used in the assay. FIG. 1A. Each binder-writer conjugate is added separately to the immobilized peptide (e.g., a peptide analyte or a fragment thereof) supplemented with a particular substrate (e.g., a nucleotide such as a dNTP, e.g., dATP only, dTTP or dUTP only, dCTP only, or dGTP only; or a single-stranded or double-stranded oligonucleotide). Upon binding of the conjugate to the immobilized peptide, the writer enzyme extends the RT by adding the substrate or a portion thereof to the 3′ hydroxyl of the RT. With extended binding, the writer enzyme can further extend the RT by adding additional units of the substrate to the extended RT, and the length of nucleotide additions depends on binding kinetics of the binder towards the particular type or class (e.g., physicochemical class) of the NTAA residue of the peptide analyte. FIG. 1B. Several diverse binder-writer fusions that recognize different physicochemical classes of NTAAs with different affinity are added as a mixture. Each binder-writer fusion is covalently conjugated with a particular substrate (specific dNTP, or specific single or double stranded oligonucleotide) via a cleavable linker, and can add only the conjugated substrate to the growing extended RT.

FIG. 2A-2B show an exemplary design of the peptide identification assay using binder-writer conjugates (encoders) shown on FIG. 1A. The assay comprises both kinetic encoding (FIG. 2A) and probabilistic decoding (FIG. 2B). FIG. 2A. An exemplary multistep encoding cycle is shown, which encodes an NTAA residue of the peptide. A peptide-recording tag (RT) conjugate is immobilized onto a solid support. An encoder that specifically binds to the NTAA localizes the writer (TdT) close to the RT. In each step, TdT catalyzes the addition of a specific nucleotide triphosphate (dNTP) to the 3′-end of the RT (generating an extended RT), whereas apyrase degrades the substrate to limit the extension duration. A washing step is performed at the end of each step to remove the step-specific encoder and reaction byproducts and minimize carryover to the next step. Then, next encoder with another specific dNTP is provided to the immobilized peptide. After all encoders are reacted with the peptide, the NTAA residue of the peptide is cleaved off, and another cycle starts with the peptide having a newly exposed NTAA residue. FIG. 2B. Each encoding cycle generates a unique nucleic acid barcode on the extended RT for each amino acid residue of the peptide. This barcode (CCCCTTTTTGGGAAA, SEQ ID NO: 26, containing subsequent x1, x2, x3, x4 stretches of specific dNTPs) is provided to a probabilistic neural network which is trained to relate the sequence of the nucleic acid barcode to amino acid identity.

FIG. 3 depicts an exemplary ProteoCode™ peptide sequencing assay with N-terminal amino acid (NTAA)-specific binding agents. (1) Peptide molecules are each associated with a DNA recording tag (RT) and attached to beads at a low molecular density, a sparsity that permits only intramolecular information transfer to occur. The peptide N-terminal amino acid (NTAA) residues are labeled with an N-terminal modification (NTM). An exemplary peptide sequence (FARDCSN; SEQ ID NO: 35) is shown. (2) Next, immobilized and labeled peptides are contacted with binding agents specific for labeled NTAA (labeled F-specific binding agent is shown). Each binding agent comprises a DNA coding tag (CT) that comprises identifying information regarding the binding agent. After binding and washing, the coding tag identifying information is transferred enzymatically to the recording tag (via extension or ligation), generating an extended RT. (3) The labeled NTAA is removed by using mild Edman-like elimination chemistry or by an engineered cleavase enzyme. The cycle 1-2-3 is repeated n times. After n cycles, the extended RT representing the n amino acids of the peptide sequence is formed and can be sequenced by NGS. A representative structure of the extended RT after 7 cycles is shown.

FIGS. 4A-4D depicts an exemplary design of a Binder-Writer-dNTP fusion complex. (FIG. 4A). A writer enzyme is expressed as a fusion of a SnoopTag (SnpT) peptide with the Writer enzyme and a C-terminal SpyCatcher (SpyC) domain. (FIG. 4B). SpyTag peptide is coupled to a nucleic acid moiety (dNTP or dN4P or dN5P) as described, and bioconjugated to the SnpT-Writer-SpyC complex via the SpyCatcher-SpyTag isopeptide bond formation to form a nucleotide-tethered writer complex. (FIG. 4C) The nucleotide-writer complex is coupled to the binder-SnpC fusion protein by isopeptide bond formation between the SnoopCatcher (SnpC) and SnoopTag (SnpT) generating a final binder-writer-dNTP complex, shown in (FIG. 4D).

FIG. 5 shows an exemplary design of triple nucleotide tethered SpyTag-based peptide. A Spytag-based peptide comprises a flexible linker attached to Spytag peptide and an N-terminal cysteine residue (CGSGSK_{N3}SGGSGGSGAHIVMVDAYKPTK; SEQ ID NO: 36) that can be labeled with thioester-derivatized nucleotide, and then with a maleimide nucleotide. The third nucleotide can be coupled with CuCAAC or SpAAC click chemistry via a lysine azide group incorporated during peptide synthesis. The three different nucleotides are designed to have phosphate length differences and or base/ribose sugar modifications to tune the incorporation rates by the template-independent polymerase (e.g., modified TdT, modified Polθ, modified PolX, etc.) used as a writer enzyme. This differential incorporation rate enables an ordered addition of nucleotides, and incorporation of a triplet code such as the ACT code to recording tag is shown. The A is incorporated from a pentaphosphate A nucleotide (dA5P), which allows for a faster reaction (incorporates faster) than d4CP or dT3P.

FIGS. 6A-6D shows exemplary designs of phosphate-tethered nucleotides (FIG. 6A). A basic structure of a triphosphate nucleotide is shown comprised of a 5′ triphosphate, a base (A, C, T, G, etc.), a five ring ribose sugar with 2′ and 3′ groups (3′ OH/2′ H for dN3P). Reversible terminators generally have a modified 3′ group such as a 3′-O azidylmethyl group. The phosphate groups are labeled with Greek letters with the innermost phosphate labeled with the 1st letter and enumerating outward. (FIG. 6B). A tetraphosphate nucleotide is shown (dN4P). (FIG. 6C) pentaphosphate nucleotide is shown (dN5P). (FIG. 6D) final binder-writer-dNTP complex.

FIGS. 7A-7B. Design of Barcoded Nucleotides (FIG. 7A). A basic structure of an oligonucleotide barcode labeled triphosphate nucleotide is shown (attached to a binder-writer via it 5′ polyphosphate moiety). An oligonucleotide barcode is attached to the base via a C5 or C7 linkage (pyrimidine or purine, respectively). The barcode can be tethered by either its 3′ or 5′ end; a 3′ tethering is illustrated. A photo-cleavable (PC) linker is shown for illustration. A 5′ phosphate moiety is useful for enzymatic ligation to the 3′ OH after deblocking. (FIG. 7B). A nucleotide structure is similar to that shown in A except designed for chemical ligation (CuAAC) to form a triazole linkage between the 5′ azide on the barcode with the 3′-O-alkyne on the 3′ position of the nucleotide.

FIGS. 8A-8B. Ligation of barcoded oligonucleotide to 3′ of incorporated nucleotide on Recording Tag (rTag) (FIG. 8A). Ligation with a ssDNA ligase, such as CircLigase, is used to ligate the 5′ phosphate terminus of the oligonucleotide barcode to the 3′ hydroxyl of the incorporated nucleotide. (FIG. 8B) After ligation, the linker between the base and the barcode is cleaved and a uracil (U) adjacent to the barcode is cleaved with USER enzyme. USER cleavage generates a 3′ phosphate group on the cleaved sequence that can be removed with alkaline phosphatase.

FIGS. 9A-9B show exemplary structures of cleavable linkers between the writer enzyme and nucleic acid moiety within binder-writer-nucleic acid moiety conjugates.

FIG. 10 shows exemplary encoding reactions performed on an immobilized set of 484 peptides (22×22 combination of different P1 and P2 residues, P1 being an NTAA residue, and P2 being the next amino acid residue after the NTAA residue (a penultimate terminal amino acid residue) of said peptides; each peptide is associated with a DNA recording tag; each cell of the array represents an encoding efficiency of the given binder that binds to a specific combination of P1-P2 residues of the target peptide) by using a free TdT enzyme (left panel) and TdT-F4R10 conjugate (right panel). The encoding efficiency was calculated as TIE_len: an average number of nucleotide bases incorporated into corresponding recording tags after 4 cycles of encoding using four individual dNTPs. Left panel shows no specificity for number of nucleotide bases incorporated into corresponding recording tags (incorporation occurs unspecifically because of a high TdT concentration used). In contrast, the right panel of FIG. 10 shows preferential encoding by the TdT-F4R10 conjugate across peptides having F, Y and W NTAA residues, consistent with the known binding selectivity of the F4R10 binder (see Example 15 for additional details).

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured. All publications, including patent documents, scientific articles and databases, referred to in this application are incorporated by reference in their entireties for all purposes. Citation of the publications or documents is not intended as an admission that any of them is pertinent prior art, nor does it constitute any admission as to the contents or date of these publications or documents.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

As used herein, the term “sample” refers to anything which may contain an analyte for which an analyte assay is desired. As used herein, a “sample” can be a solution, a suspension, liquid, powder, a paste, aqueous, non-aqueous or any combination thereof. In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom.

As used herein, the term “peptide” encompasses peptides, polypeptides, and proteins, and refers to a molecule comprising a chain of two or more amino acids joined by peptide bonds. In some embodiments, a peptide comprises 2 to 50 amino acids. A component of a peptide may comprise a single amino acid residue (such as a terminal amino acid residue), two or more amino acid residues, a part or parts of a peptide, a part or parts of a peptide that is/are recognized by a binding agent (for example, an epitope recognized by an antibody), or the whole peptide. In some embodiments, a peptide does not comprise a secondary, tertiary, or higher structure. In some embodiments, the peptide is a protein. In some embodiments, a protein comprises 30 or more amino acids. In some embodiments, in addition to a primary structure, a protein comprises a secondary, tertiary, or higher structure. The amino acids of the peptides are most typically L-amino acids, but may also be D-amino acids, modified amino acids, amino acid analogs, amino acid mimetics, or any combination thereof. Peptides may be naturally occurring, synthetically produced, or recombinantly expressed. Peptides may be synthetically produced, isolated, recombinantly expressed, or be produced by a combination of methodologies as described above. Peptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring (or natural) types of amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). These 20 amino acids form 20 specific types of amino acid residues present in peptides. An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, n-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids. The term “amino acid residue” refers to an amino acid incorporated into a peptide that forms peptide bond(s) with neighboring amino acid(s).

As used herein, the term “post-translational modification” refers to modifications that occur on a peptide after its translation, e.g., translation by ribosomes, is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. The term post-translational modification can also include peptide modifications that include one or more detectable labels.

The term “detectable label” as used herein refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Examples of detectable labels include a dye, a fluorophore, a chromophore, a fluorescent nanoparticle (e.g., quantum dot), a radiolabel, an enzyme (e.g., alkaline phosphatase, luciferase or horseradish peroxidase), or a chemiluminescent or bioluminescent molecule.

As used herein, the term “linker” refers to one or more of a nucleotide, a nucleotide analog, an amino acid, a peptide, a peptide, a polymer, or a non-nucleotide chemical moiety that is used to join two molecules. A linker may be used to join a recording tag with a peptide, a peptide with a support, a recording tag with a solid support, etc. In certain embodiments, a linker joins two molecules via enzymatic reaction or chemistry reaction (e.g., a click chemistry reaction). In certain embodiments, the nucleic acid recording tag is associated directly or indirectly to the peptide analyte via a non-nucleotide chemical moiety.

The terminal amino acid at one end of a peptide or peptide chain that has a free amino group is referred to herein as the “N-terminal amino acid” (NTAA). The terminal amino acid at the other end of the chain that has a free carboxyl group is referred to herein as the “C-terminal amino acid” (CTAA). In certain embodiments, an NTAA, CTAA, or both may be modified or labeled with a moiety or a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a peptide, a coding tag, a plurality of coding tags from an encoding cycle, a sample peptides, a set of samples, peptides within a compartment (e.g., droplet, bead, or separated location), peptides within a set of compartments, a fraction of peptides, a spatial region or set of spatial regions. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual peptide, sample, library, etc. A barcode can also be used for deconvolution of a collection of coding tags configured to react selectively with a specific type of amino acid residue(s) present in an immobilized peptide.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag. Sp′ refers to spacer sequence complementary to Sp. A spacer sequence may comprise sufficient number of bases to anneal to a complementary spacer sequence in a recording tag to initiate a primer extension (also referred to as polymerase extension) reaction, or provide a “splint” for a ligation reaction.

As used herein, the term “recording tag” refers to a nucleic acid molecule, or a sequenceable polymer molecule (see, e.g., Niu et al., 2013, Nat. Chem. 5:282-292; Roy et al., 2015, Nat. Commun. 6:7237) that is associated with a peptide during the assay and accumulates information related to peptide's amino acid identity. This information is added to the growing recording tag by a writer enzyme during the assay, so that a single NTAA residue of the peptide generates a nucleotide string (barcode) on the recording tag, which can uniquely identify the assayed NTAA residue. A recording tag may be directly linked to a peptide, linked to a peptide via a multifunctional linker, or associated with a peptide by virtue of its proximity (or co-localization) on a support. A recording tag may be associated via its 5′ end or 3′ end or at an internal site, as long as the linkage is compatible with the method used to generate barcode information encoding the NTAA residue on the recording tag. A recording tag may further comprise other functional components, e.g., a universal priming site, unique molecular identifier, another barcode (e.g., a sample barcode, a fraction barcode, spatial barcode, a compartment tag, etc.).

As used herein, the term “unique molecular identifier” or “UMI” refers to a nucleic acid molecule of about 3 to about 40 bases in length providing a unique identifier tag for each peptide to which the UMI is linked. A peptide UMI can be used to computationally deconvolute sequencing data from a plurality of extended recording tags to identify extended recording tags that originated from an individual peptide. A peptide UMI can be used to accurately count originating peptide molecules by collapsing NGS reads to unique UMIs.

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing.

As used herein, the term “extended recording tag” refers to a recording tag on which information encoding NTAA residue of the associated immobilized peptide (a barcode region) is generated following binding of the binder to the NTAA of the peptide. Barcode region can be generated on the recording tag by a single writer enzyme or by different writer enzymes that catalyze covalent addition of nucleic acid moiety to the 3′ hydroxyl of the nucleic acid recording tag. An extended recording tag may comprise information encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more NTAA residues (each NTAA residue is represented by a different barcode). The sequence of an extended recording tag may reflect the sequential order of the amino acid residues of the associated peptide, since the amino acid residues are configured to be processed sequentially (in each encoding cycle, current NTAA residue of the peptide is processed and then cleaved off to expose new NTAA residue to be processed in the next cycle).

As used herein, the term “binding agent”, or “binder”, refers to a nucleic acid molecule (e.g., an aptamer), a polypeptide, a carbohydrate, or a macromolecule that binds to, associates, unites with, recognizes, or combines with a binding target, e.g., a peptide or a component or feature of a peptide. A binding agent may form a covalent association or non-covalent association with the peptide or component or feature of a peptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. In preferred embodiments, a binding agent binds specifically to a chemically modified NTAA residue of the target peptide over a non-modified or unlabeled NTAA residue.

As used herein, the term “selectivity” refers to the ability of a binding agent to preferentially bind to one or to several amino acid residues of a peptide analyte, optionally modified with a chemical modification. In preferred embodiments, “selectivity” describes preferential binding of a binding agent to a single terminal amino acid residue, optionally modified with a chemical modification. In some embodiments, a binding agent may exhibit selective binding to a particular amino acid residue or modified amino acid residue. In some embodiments, a binding agent may exhibit selective binding to a particular class or type of amino acid residues or modified amino acid residues. In some embodiments, a binding agent may exhibit particular binding kinetics (e.g., higher association rate constant and/or lower dissociation rate constant) to a particular class or type of amino acid residues or modified amino acid residues, compared to other amino acid residues or modified amino acid residues. In some embodiments, a binding agent may exhibit selective binding to a component or feature of a peptide analyte (e.g., a binding agent may selectively bind to one of the 20 possible natural amino acid residues modified with a chemical modification, and bind with very low affinity or not at all to the other 19 natural amino acid residues modified with the same chemical modification). In other embodiments, binding agent may exhibit less selective binding, where the binding agent is capable of binding or configured to bind to a plurality of components or features of a peptide analyte (e.g., a binding agent may bind with similar affinity to two or more different amino acid residues modified with a chemical modification). In preferred embodiments, a binding agent is conjugated with a writer enzyme, which may be joined to the binding agent by a linker such that both the binding agent and the writer enzyme remain functional within the conjugate. In some embodiments, selectivity of each binding agent conjugated with the writer enzyme towards NTAA resides or modified NTAA resides of peptide analytes is determined in advance, before performing contacting steps of the disclosed methods.

In some embodiments, a binding agent comprises a polypeptide, e.g., an antibody fragment or an engineered polypeptide binder. In other embodiments, a binding agent comprises an aptamer. In some embodiments, the polypeptide binding agent and the writer enzyme are parts of a fusion molecule such as a fusion polypeptide.

As used herein, the term “binding kinetics” describes the speed at which a binding agent binds to and dissociates from a binding partner, such as a peptide immobilized on a support. Binding kinetics describes a dynamic binding interaction between two molecules, typically expressed as Ka (the rate of association), Kd (rate of disassociation) and KD (equilibrium dissociation constant). Kd describes the rate at which the interacting molecules disassociate after forming a complex.

As used herein, the term “writer enzyme”, or “writer”, refers to an enzyme capable of catalyzing covalent addition of a nucleic acid moiety to a terminus of a nucleic acid recording tag. In preferred embodiments, the writer enzyme is or comprises a template-independent polymerase (such as Terminal deoxynucleotidyl Transferase (TdT), DNA Polymerase Mu, DNA Polymerase theta, or a variant thereof), a DNA ligase (such as T4 DNA ligase), or a RNA ligase (such as T4 RNA ligase). In some embodiments, a writer enzyme is a functional fragment or derivative of a natural writer enzyme, and the fragment or derivative retains or substantially retains the activity of the natural writer enzyme for catalyzing covalent addition of a nucleic acid moiety to a terminus of a nucleic acid recording tag. In some embodiments, the fragment or derivative retains at least 70%, 80%, 85%, 86%, 87%, 88%, 89%, 90%, 91%, 92%, 93%, 94%, 95%, 96%, 97%, 98%, 99% or more of the activity of the natural writer enzyme for catalyzing covalent addition of a nucleic acid moiety to a terminus of a nucleic acid recording tag.

As used herein, the term “conjugate” refers to a macromolecule that comprises a binding agent and a writer enzyme joined together by a linker (e.g., a flexible linker). In some embodiments, the linker is a peptide. In other embodiments, the linker is a non-peptide moiety and a non-nucleic acid moiety. In preferred embodiments, the linker does not interfere with functional activities of both the binding agent and the writer enzyme (or subunits or domains thereof) which it joins. One example is the conjugate that comprises a binding agent conjugated via a linker to a writer enzyme, wherein said binding agent is configured to bind to a peptide joined to a support and associated with a nucleic acid recording tag, and said writer enzyme is configured to catalyze covalent addition of a nucleic acid moiety onto a terminus of said recording tag.

As used herein, the term “support” can include a solid support and/or a solid surface and include any suitable material such as a solid material, including porous and non-porous materials, to which a peptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a silicon wafer chip, a flow through chip, a flow cell, a biochip including signal transducing electronics, a channel, a microtiter well, an ELISA plate, a spinning interferometry disc, a nitrocellulose-based polymer surface, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality. In some embodiments, the nucleic acid molecule or oligonucleotide is a modified oligonucleotide. In some embodiments, the nucleic acid molecule or oligonucleotide is backbone modified, sugar modified, or nucleobase modified.

As used herein, the term “nucleic acid moiety” refers to a nucleotide, dinucleotide, trinucleotide, or a derivative thereof, which can be added to a terminus of a nucleic acid recording tag by a writer enzyme to generate an extended nucleic acid recording tag. A nucleic acid moiety can be a nucleotide moiety. A nucleic acid moiety can comprise a nucleoside linked to one or more phosphate groups. In some embodiments, a nucleic acid moiety comprises one, two, three, four or five phosphate groups linked to a nucleoside. A nucleoside within a nucleic acid moiety may be either a DNA nucleoside or a RNA nucleoside. The structure of nucleic acid moiety allows performing sequencing of the nucleic acid moiety within the extended nucleic acid recording tag (e.g., after the nucleic acid moiety is added to the terminus of the nucleic acid recording tag).

As used herein, “sequencing” means the determination of the order of nucleotides in a nucleic acid molecule, a modified nucleic acid molecule, or a sample of modified nucleic acid molecules. “Peptide sequencing” means the determination of the identity and order of at least a portion of amino acids in the peptide molecule or in a sample of peptide molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche.

As used herein, “identifying” a peptide means to predict identity of the peptide with a certain probability. It can be done by identifying a component (e.g., one or more amino acid residues) of the peptide. It can also be done by predicting certain amino acid residues of the peptide and their positions with certain probability, thus creating a peptide signature, and then matching bioinformatically the resulted peptide signature with corresponding signatures of peptides that may be present in the sample (e.g., by matching the peptide signature with peptide sequences from a proteomic or genomic database). For example, in some embodiments, existing selectivity of a binder is not enough to determine the NTAA residue to which the binder is bound with certainty. In these cases, identity of the NTAA residue can be determined with certain probability (such as being D, E or H and not A, G, I or L). Subsequent similar determination of adjacent amino acid residues creates an array of possible variants for the peptide based on variants in the assayed amino acid residues, and by matching this array of variants with theoretical possibilities determined from a proteomic or genomic database, it can be narrowed down to a particular sequence, if enough amino acid residues were assayed.

The term “sequence identity” is a measure of identity between peptides at the amino acid level, and a measure of identity between nucleic acids at nucleotide level. The peptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website.

The terms “corresponding to position(s)” or “position(s) . . . with reference to position(s)” of or within a peptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the peptide upon alignment with the disclosed sequence using a standard alignment algorithm, such as the BLAST algorithm (NCBI). One skilled in the art can identify any given amino acid residue in a given peptide at a position corresponding to a particular position of a reference sequence, such as set forth in the Sequence Listing, by performing alignment of the peptide sequence with the reference sequence (for example, by using BLASTP publicly available through the NCBI website), matching the corresponding position of the reference sequence with the position in peptide sequence and thus identifying the amino acid residue within the peptide.

The term “peptide bond” as used herein refers to a chemical bond formed between two molecules (such as two amino acids) when the carboxyl group of one molecule reacts with the amino group of the other molecule, releasing a water molecule (H2O).

The term “unmodified” (also “wild-type” or “native”) as used herein is used in connection with biological materials such as nucleic acid molecules and proteins (e.g., cleavase), refers to those which are found in nature and not modified by human intervention.

The term “modified” or “engineered” (or “variant”, or “mutant”) as used in reference to nucleic acid molecules and protein molecules, e.g., an engineered binder or engineered cleavase enzyme, implies that such molecules are created by human intervention and/or they are non-naturally occurring. The variant, mutant or engineered binder or cleavase is a peptide having an altered amino acid sequence, relative to an unmodified or wild-type protein, such as starting scaffold, or a portion thereof. An engineered enzyme is a peptide which differs from a wild-type enzyme scaffold sequence, or a portion thereof, by one or more amino acid substitutions, deletions, additions, or combinations thereof. An engineered binder generally exhibits at least 70%, 80%, 85%, 86%, 87%, 88%, 89%, 90%, 91%, 92%, 93%, 94%, 95% 96%, 97%, 98%, 99% or more sequence identity to a corresponding wild-type starting protein scaffold. Non-naturally occurring amino acids as well as naturally occurring amino acids are included within the scope of permissible substitutions or additions.

In some embodiments, variants of an engineered binder or cleavase displaying only non-substantial or negligible differences in structure can be generated by making conservative amino acid substitutions in the engineered binder or cleavase. By doing this, further engineered binder variants that comprise a sequence having at least 90% (90%, 91%, 92%, 93%, 94%, 95%, 96%, 97%, 98%, and 99%) sequence identity with the initial engineered binder sequences can be generated, retaining at least one functional activity of the engineered binder, e.g., ability to specifically bind to the N-terminally modified target peptide. Examples of conservative amino acid changes are known in the art. Examples of non-conservative amino acid changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e g., glycine. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The terms “specifically binding” and “specifically recognizing” are used interchangeably herein and generally refer to an engineered binder that binds to a cognate target peptide or a portion thereof more readily than it would bind to a random, non-cognate peptide. The term “specificity” is used herein to qualify the relative affinity by which an engineered binder binds to a cognate target peptide. Specific binding typically means that an engineered binder binds to a cognate target peptide at least twice more likely that to a random, non-cognate peptide (a 2:1 ratio of specific to non-specific binding). Non-specific binding refers to background binding, and is the amount of signal that is produced in a binding assay between an engineered binder and an N-terminally modified target peptide when the modified NTAA residue cognate for the engineered binder is not present at the N-terminus of the target peptide. In some embodiments, specific binding refers to binding between an engineered binder and an N-terminally modified target peptide with a dissociation constant (Kd) of 200 nM or less.

In some embodiments, binding specificity between an engineered binder and an N-terminally modified target peptide is predominantly or substantially determined by interaction between the engineered binder and the modified NTAA residue of the N-terminally modified target peptide, which means that there is only minimal or no interaction between the engineered binder and the penultimate terminal amino acid residue (P2) of the target peptide, as well as other residues of the target peptide. In some embodiments, the engineered binder binds with at least 5-fold higher affinity to the modified NTAA residue of the target peptide than to any other region of the target peptide. In some embodiments, the engineered binder binds with at least 5-fold lower Kd (dissociation constant) to the modified NTAA residue of the target peptide than to any other region of the target peptide. In some embodiments, the engineered binder has a substrate binding pocket with certain size and/or geometry matching the size and/or geometry of the modified NTAA residue of the N-terminally modified target peptide, to which the engineered binder specifically binds to. In such embodiments, the modified NTAA residue occupies a volume encompassing a substrate binding pocket of the engineered binder that effectively precludes the P2 residue of the target peptide from entering into the substrate binding pocket or interacting with affinity-determining residues of the engineered binder. In some embodiments, the engineered binder specifically binds to N-terminally modified target peptides, wherein the target peptides share the same modified NTAA residue that interacts with the engineered binder, but have different P2 residues. In some embodiments, the engineered binder is capable of specifically binding to each N-terminally modified target peptide from a plurality of N-terminally modified target peptides, wherein the plurality of N-terminally modified target peptides contains at least 3, at least 5, or at least 10 N-terminally modified target peptides that were modified with the same N-terminal modifier agent, have the same modified NTAA residue, and have different P2 residues. Thus, in preferred embodiments, the engineered binder possesses binding affinity towards one or more of the modified NTAA residues of the N-terminally modified target peptide, but has little or no affinity towards P2 or other residues of the target peptide.

As used herein “amplification” refers to any in vitro method for increasing the number of copies of a nucleotide sequence with the use of a DNA polymerase. Nucleic acid amplification results in the incorporation of nucleotides into a DNA molecule or primer thereby forming a new DNA molecule complementary to a DNA template. The formed DNA molecule and its template can be used as templates to synthesize additional DNA molecules.

The terms “hybridization” and “hybridizing” refers to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used. In the present invention, the term “hybridization” refers particularly to hybridization of an oligonucleotide to a template molecule.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

It is understood that aspects and embodiments of the invention described herein include “consisting of” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Disclosure of Different Embodiments of the Invention.

In one embodiment, a method for analyzing a peptide is provided, wherein the peptide and an associated nucleic acid recording tag are joined to a support, the method comprising: a) contacting the peptide with a first composition comprising a first conjugate and a first nucleic acid moiety, wherein the first conjugate comprises a first binding agent that binds to the peptide, wherein the first binding agent is conjugated to a first writer enzyme that catalyzes covalent addition of the first nucleic acid moiety to a terminus of the nucleic acid recording tag to generate an extended nucleic acid recording tag joined to the support; b) contacting the peptide with a second composition comprising a second conjugate and a second nucleic acid moiety, wherein the second conjugate comprises a second binding agent that binds to the peptide, wherein the second binding agent is conjugated to a second writer enzyme capable of catalyzing covalent addition of the second nucleic acid moiety to a terminus of the extended nucleic acid recording tag to generate a further extended nucleic acid recording tag joined to the support; and c) analyzing the further extended nucleic acid recording tag to obtain information regarding binding kinetics and/or selectivity of the first binding agent binding to the peptide and information regarding binding kinetics and/or selectivity of the second binding agent binding to the peptide, thereby analyzing the peptide

In another embodiment, a method for analyzing a peptide is provided, wherein the peptide and an associated nucleic acid recording tag are joined to a support, the method comprising the steps of: a) contacting the peptide with a mixture of compositions comprising a first composition and a second composition, wherein (i) the first composition comprises a first conjugate and a first nucleic acid moiety; the first conjugate comprises a first binding agent that binds to the peptide; the first binding agent is conjugated to a first writer enzyme that catalyzes covalent addition of the first nucleic acid moiety to a terminus of the nucleic acid recording tag; and the first nucleic acid moiety is tethered to and controllably cleavable from the first writer enzyme; (ii) the second composition comprises a second conjugate and a second nucleic acid moiety; the second conjugate comprises a second binding agent that binds to the peptide; the second binding agent is conjugated to a second writer enzyme that catalyzes covalent addition of the second nucleic acid moiety to the terminus of the nucleic acid recording tag; and the second nucleic acid moiety is tethered to and controllably cleavable from the second writer enzyme, thereby generating an extended nucleic acid recording tag joined to the support, wherein the extended nucleic acid recording tag comprises covalent addition of the first and/or second nucleic acid moiety; b) cleaving the first nucleic acid moiety from the first writer enzyme and/or cleaving the second nucleic acid moiety from the second writer enzyme, thereby releasing the first and/or second writer enzyme from the extended nucleic acid recording tag; c) optionally, repeating steps (a) and (b) one or more times to generate a further extended nucleic acid recording tag joined to the solid support; and d) analyzing the extended nucleic acid recording tag or the further extended nucleic acid recording tag and obtaining information regarding binding kinetics and/or selectivity of the binding agents bound to the peptide, thereby analyzing the peptide.

In yet another embodiment, a method for identifying a component of a peptide is provided, the method comprising the steps of: (a) providing the peptide and an associated nucleic acid recording tag joined to a solid support; (b) contacting the peptide with a first composition comprising a first conjugate and a first nucleic acid moiety, wherein the first conjugate comprises a first binding agent capable of binding to the peptide, wherein the first binding agent is conjugated to a writer enzyme capable of catalyzing covalent addition of the first nucleic acid moiety to a terminus of the nucleic acid recording tag; (c) following the binding of the first conjugate to the peptide, allowing the writer enzyme of the first conjugate to catalyze covalent addition of the first nucleic acid moiety to the terminus of the nucleic acid recording tag to generate an extended nucleic acid recording tag joined to the solid support; (d) contacting the peptide with a second composition comprising a second conjugate and a second nucleic acid moiety, wherein the second conjugate comprises a second binding agent capable of binding to the peptide, wherein the second binding agent is conjugated to a writer enzyme capable of catalyzing covalent addition of the second nucleic acid moiety onto a terminus of the extended nucleic acid recording tag; (e) following the binding of the second conjugate to the peptide, allowing the writer enzyme of the second conjugate to catalyze covalent addition of the second nucleic acid moiety to the terminus of the extended nucleic acid recording tag to generate a further extended nucleic acid recording tag joined to the solid support; (f) optionally, repeating steps (d)-(e) one or more times by replacing the second composition with a third or higher order composition comprising a third or higher order conjugate and a third or higher order nucleic acid moiety, wherein the third or higher order conjugate comprises a third or higher order binding agent capable of binding to the peptide conjugated via a flexible linker to a writer enzyme capable of catalyzing covalent addition of the third or higher order nucleic acid moiety onto a terminus of the extended or further extended nucleic acid recording tag; and by allowing the writer enzyme of the third or higher order conjugate to catalyze covalent addition of the third or higher order nucleic acid moiety to the terminus of nucleic acid recording tag extended after step (e) or after previous addition(s) to generate a further extended nucleic acid recording tag joined to the solid support; and (g) analyzing the further extended nucleic acid recording tag and obtaining information regarding binding kinetics or selectivity of the first binding agent binding to the peptide and information regarding binding kinetics or selectivity of the second binding agent binding to the peptide, thereby identifying a component of the peptide.

Another embodiment of this disclosure provides a method for identifying a component of a peptide, the method comprising the steps of (a) providing the peptide and an associated nucleic acid recording tag joined to a solid support; (b) contacting the peptide with a mixture comprising a first composition, a second composition, and, optionally, a third or higher order composition, wherein (i) the first composition comprises a first conjugate and a first nucleic acid moiety; the first conjugate comprises a first binding agent capable of binding to the peptide; the first binding agent is conjugated to a writer enzyme capable of catalyzing covalent addition of the first nucleic acid moiety to a terminus of the nucleic acid recording tag; and the first nucleic acid moiety is covalently tethered to the writer enzyme via a linker comprising a selectively cleavable linkage; (ii) the second composition comprises a second conjugate and a second nucleic acid moiety; the second conjugate comprises a second binding agent capable of binding to the peptide; the second binding agent is conjugated to a writer enzyme capable of catalyzing covalent addition of the second nucleic acid moiety to a terminus of the nucleic acid recording tag; and the second nucleic acid moiety is covalently tethered to the writer enzyme via a linker comprising a selectively cleavable linkage; (iii) the third or higher order composition comprises a third or higher order conjugate and a third or higher order nucleic acid moiety; the third or higher order conjugate comprises a third or higher order binding agent capable of binding to the peptide; the third or higher order binding agent is conjugated to a writer enzyme capable of catalyzing covalent addition of the third or higher order nucleic acid moiety to a terminus of the nucleic acid recording tag; and the third or higher order nucleic acid moiety is covalently tethered to the writer enzyme via a linker comprising a selectively cleavable linkage; (c) following binding of the first conjugate, the second conjugate or the third or higher order conjugate to the peptide, allowing the writer enzyme of the conjugate bound to the peptide to catalyze covalent addition of the nucleic acid moiety covalently tethered to the writer enzyme to the terminus of the nucleic acid recording tag to generate an extended nucleic acid recording tag joined to the solid support; (d) cleaving the selectively cleavable linkage and releasing the writer enzyme from the nucleic acid moiety on the terminus of the extended nucleic acid recording tag; (e) optionally, repeating steps (b)-(d) one or more times to generate a further extended nucleic acid recording tag joined to the solid support; and (f) analyzing the extended nucleic acid recording tag or the further extended nucleic acid recording tag and obtaining information regarding binding kinetics or selectivity of the binding agent(s) bound to the peptide, thereby identifying a component of the peptide.

In yet another embodiment of the invention, a method for identifying a peptide is provided, comprising the steps of (a) providing the peptide and an associated nucleic acid recording tag joined to a solid support; (b) contacting the peptide with a first composition comprising a first conjugate and a first nucleic acid moiety, wherein the first conjugate comprises a first binding agent capable of binding to the peptide, wherein the first binding agent is conjugated to a writer enzyme capable of catalyzing covalent addition of a nucleic acid moiety to a terminus of the nucleic acid recording tag; (c) following the binding of the first conjugate to the peptide, allowing the writer enzyme of the first conjugate to catalyze covalent addition of the first nucleic acid moiety to the terminus of the nucleic acid recording tag to generate an extended nucleic acid recording tag joined to the solid support; (d) repeating steps (b) and (c) one or more times by replacing the first composition with a second or higher order composition comprising a second or higher order conjugate and a second or higher order nucleic acid moiety, wherein the second or higher order conjugate comprises a second or higher order binding agent capable of binding to the peptide, wherein the second or higher order binding agent is conjugated to a writer enzyme capable of catalyzing covalent addition of a nucleic acid moiety onto a terminus of the nucleic acid recording tag; and by allowing the writer enzyme of the second or higher order conjugate to catalyze covalent addition of the second or higher order nucleic acid moiety to the terminus of the nucleic acid recording tag extended after step (c) or after previous addition(s) to generate a further extended nucleic acid recording tag joined to the solid support; and (e) analyzing the further extended nucleic acid recording tag and obtaining information regarding binding kinetics or selectivity of the first binding agent binding to the peptide and information regarding binding kinetics or selectivity of the second or higher order binding agent, thereby identifying the peptide.

In yet another embodiment, provided herein is a conjugate which comprises a binding agent conjugated via a first linker to a writer enzyme, wherein said conjugate is configured to bind to a peptide, wherein the peptide and an associated nucleic acid recording tag are joined to a support, said binding agent is configured to bind to said peptide, and said writer enzyme is configured to catalyze covalent addition of a nucleic acid moiety onto a terminus of said nucleic acid recording tag.

In yet another embodiment, provided herein is a composition comprising one, two or more conjugates, wherein each conjugate comprises a binding agent conjugated via a first linker to a writer enzyme, wherein each binding agent is configured to bind to a peptide, wherein the peptide and an associated nucleic acid recording tag are joined to a support, and each writer enzyme is configured to catalyze covalent addition of a nucleic acid moiety onto a terminus of each nucleic acid recording tag.

Various embodiments apply equally to the aspects provided herein but will for the sake of brevity be recited only once. Thus, various of the following embodiments apply equally to the three main embodiments recited above. It is also to be understood that, while methods are described in the context of a peptide, such methods are also directed to identifying components of plurality of peptides. Thus, in many aspects, the methods comprise a first step of providing peptides, wherein each peptide is associated with a recording tag immobilized on a solid support, followed by a second step of contacting at least a subset of the peptides with the first composition, or a mixture of compositions.

In preferred embodiments, the writer enzyme is a template-independent polymerase, a DNA ligase, or a RNA ligase.

In preferred embodiments, the analyzing step comprises a nucleic acid sequencing method.

In some embodiments, the first binding agent and/or the second binding agent is each independently capable of specific binding to one or more unmodified or modified terminal amino acid residues, optionally wherein the one or more unmodified or modified terminal amino acid residues are unmodified or modified NTAA residue(s).

In some embodiments, the first binding agent binds to a terminal amino acid (TAA) or a modified TAA of the peptide, and the second binding agent binds to the terminal amino acid (TAA) or the modified TAA of the peptide. TAA residue can be a NTAA or CTAA residue of the peptide.

In some embodiments, the disclosed methods further comprise cleaving the peptide to generate a cleaved peptide, thereby removing the TAA or the modified TAA to expose a new TAA, and, optionally, modifying the new TAA to yield a newly modified TAA.

In some embodiments, during the analyzing step, an artificial intelligence (AI) model, e.g., an AI model employing probabilistic neural networks (PNN), is applied to calculate probabilities of occurrence of specific types of amino acid residues in corresponding places in amino acid sequence of the peptide based on a nucleotide sequence of the extended and/or the further extended nucleic acid recording tag(s).

In some embodiments, the writer enzyme catalyzes covalent addition of a nucleic acid moiety to the 3′ hydroxyl of the nucleic acid recording tag.

In some embodiments, the covalent addition of a nucleic acid moiety to the terminus of the nucleic acid recording tag occurs for a controlled amount of time.

In some embodiments, the conjugate further comprises a nucleic acid moiety that is covalently tethered to the writer enzyme via a second linker comprising a selectively cleavable linkage. The second linker may comprise, for example, alkyl, PEG, or PEO moiety with 2-18 chain lengths, or any other suitable linker.

In preferred embodiments, the binding agent within the conjugate is configured to bind to a terminal amino acid (TAA) or a modified TAA of the peptide.

In some embodiments, each binding agent within the two or more of the conjugates of the disclosed composition is configured to bind to a terminal amino acid (TAA) or a modified TAA of the peptide. In preferred embodiments, each binding agent within the two or more of the conjugates of the disclosed composition has a different selectivity towards terminal amino acids or modified terminal amino acids of peptides. In some embodiments, the disclosed composition comprises 3, 4, 5, 6, 7, 8, 9, 10 or more conjugates. In some embodiments, the disclosed composition comprises 3, 4, 5, 6, 7, 8, 9, 10 or more conjugates that have the essentially same writer enzyme, and have different binding agent. In some of these embodiments, different binding agent have different selectivities and/or binding kinetics towards terminal amino acid residues or modified terminal amino acid residues of peptides. In some of these embodiments, different binding agent have different specificities and/or binding kinetics towards terminal amino acid residues or modified terminal amino acid residues of peptides.

In preferred embodiments, each writer enzyme within the two or more of the conjugates of the disclosed composition is essentially the same (e.g., writer enzymes comprise the same amino acid sequences). In some embodiments, writer enzymes within the two or more of the conjugates of the disclosed composition are different.

In some embodiments, the peptide is obtained by fragmenting a protein from a biological sample. Examples of biological samples include, but are not limited to cells (both primary cells and cultured cell lines), cell lysates or extracts, cell organelles or vesicles, including exosomes, tissues and tissue extracts; biopsy; fecal matter; bodily fluids (such as blood, serum, plasma, urine, lymph). A peptide, peptide, protein, or protein complex may comprise a standard, naturally occurring amino acid, a modified amino acid (e.g., post-translational modification), an amino acid analog, an amino acid mimetic, or any combination thereof.

In some embodiments, the peptide is obtained by fragmenting a protein from a biological sample, and immobilized on a solid support and associated with a recording tag by methods disclosed in US 2022/0049246 A1, incorporated herein. In these embodiments, the solid support comprises a plurality of DNA hairpins immobilized on the solid support and configured to capture via hybridization the one or more nucleic acid recording tags associated with the peptide(s).

In some embodiments, peptide immobilization is performed according to the following method: attaching a peptide analyte to a bait nucleic acid to generate a nucleic acid-analyte chimera; bringing the nucleic acid-peptide chimera into proximity with a solid support by hybridizing the bait nucleic acid in the nucleic acid-peptide chimera to a capture nucleic acid attached to the solid support; and covalently coupling the nucleic acid-peptide chimera to the solid support; wherein a plurality of the nucleic acid-peptide chimeras is coupled on the solid support and any adjacently coupled nucleic acid-peptide chimeras are spaced apart from each other at an average distance of about 50 nm or greater.

In preferred embodiments, the length of the immobilized peptide is greater than 4 amino acids. Peptides of 4 amino acids or less are unlikely to be used for identification of a protein, from which they originate. In some other embodiments, the length of the immobilized peptide is greater than 10 amino acids.

In some embodiments of the disclosed methods, the contacting steps (b) and (d) are performed in sequential order. In these embodiments, the second composition is added to the peptide sequentially, after the nucleic acid recording tag is extended. Preferably, before the second composition is added to the peptide, the first composition is washed away. Likewise, the third composition is added to the peptide after the nucleic acid recording tag is extended after the second composition was added. Preferably, before the third composition is added to the peptide, the second composition is washed away. A washing buffer used to wash away the first, second or higher order composition does not compromise integrity of the peptide, nucleic acid recording tag, writer enzyme or binding agent. In some embodiments, the washing buffer comprises a mild detergent that does not interfere with the extension reaction catalyzed by the writer enzyme, such as Tween 20, Triton X-100, NP-40 and the like. One example of the washing buffer include a PBST buffer (phosphate-buffered saline with 0.05% to 0.1% Tween 20 detergent).

In other embodiments, the contacting steps (b) and (d) are performed at the same time. In these embodiments, the first composition is added to the peptide simultaneously with the second composition, and, optionally, simultaneously with the third or higher order (such as fourth, fifth, etc.) compositions. In these embodiments, the first, second, third and optionally higher order conjugates compete for binding to the peptide, and preferably, to a component of the peptide that needs to be identified. In some of these embodiments, the conjugates compete for binding to an N-terminal amino acid (NTAA) residue of the peptide, preferably labeled with a modifying reagent; information regarding binding kinetics and/or selectivity of the binding agent of the conjugate bound to the (labeled) NTAA residue of the peptide is encoded by extending nucleic acid recording tag; and this information is obtained during the analysis step to provide structural information regarding the (labeled) NTAA residue of the peptide, which leads to the NTAA residue identification. In other embodiments, the conjugates compete for binding to a component (comprising one or more amino acid residues, an epitope, etc.) of the peptide; information regarding binding kinetics and/or selectivity of the binding agent of the conjugate bound to the component of the peptide is encoded by extending nucleic acid recording tag; and this information is obtained during the analysis step to provide structural information regarding the component of the peptide, which leads to the component identification. Preferably, in these embodiments, the first, second and, optionally, higher order compositions comprise a nucleic acid moiety covalently tethered to the writer enzyme (e.g., a template-independent polymerase, a DNA ligase, or a RNA ligase) via a linker comprising a selectively cleavable linkage. Thus, identity of the nucleic acid moiety installed on the nucleic acid recording tag during the extension step is linked to the identity of the binding agent bound to the peptide (or to a component of the peptide), which allows for decoding the identity of the binding agent, and the correspondent component of the peptide analyte to which binding agent was bound, based on analysis of the nucleic acid recording tag (such as by sequencing of the nucleic acid recording tag).

In some embodiments, a plurality of peptides is provided at step (a), each peptide from the plurality of peptides is independently associated with a nucleic acid recording tag (which can be the same or different for any two or more molecules of any one or more peptides of the plurality of peptides) joined to a solid support, and wherein the plurality of peptides is contacted with the first composition at step (b) (at the first contacting step) and with the second composition at step (d) (at the second contacting step).

In some embodiments, the plurality of peptides comprises at least 10, 20, 50, 100, 200, 500, 1000, 10000, 100000, 1,000,000 or more peptides. These peptides can be processed in parallel. In some embodiments, at least 10, 20, 50, 100, 200, 500, 1,000, 10,000, 100000, 1,000,000 or more peptides are identified during the analyzing step. In some embodiments, the plurality of peptides comprises at least 10, 20, 50, 100, 200, 500, 1,000, 10,000, or more different peptides are identified during the analyzing step.

In some embodiments, the writer enzyme is a template-independent polymerase, a DNA ligase, or a RNA ligase.

In some embodiments, the template-independent polymerase is a Terminal deoxynucleotidyl Transferase (TdT).

In some embodiments, the template-independent polymerase is a variant of TdT that possesses certain advantages over wild type TdT, such as improved thermostability or the ability to incorporate modified nucleic acid moieties (e.g., nucleotide moieties).

In some embodiments, the template-independent polymerase is a variant of TdT that is capable of a controlled addition of a nucleic acid moiety to the terminus of the nucleic acid recording tag, as disclosed in US 2020/0263152 A1.

In some embodiments, the template-independent polymerase is a variant of TdT that is engineered to accommodate modified nucleic acid moieties (e.g., modified nucleotide moieties), such as 3′-OH modified nucleic acid moieties (e.g., 3′-OH modified nucleotide moieties), or nucleic acid moieties (e.g., nucleotide moieties) modified in the gamma phosphate group. Such TdT variants are disclosed, for example, in US 2019/0211315 A1, U.S. Pat. Nos. 10,752,887 B2, 10,774,316 B2, 11,208,637 B2, US 2019/0275492 A1, incorporated herein.

In some embodiments, the template-independent polymerase is a thermostable variant of TdT, as disclosed in US 2021/0355460 A1. The advantage of a thermostable TdT is that it can work efficiently at an elevated temperature, which can reduce formation of the secondary structure at the terminus of the nucleic acid recording tag. It is known that secondary structure formation may reduce efficiency of nucleotide addition by TdT.

In some embodiments, the template-independent polymerase is a variant of a poly(A) polymerase (PAP) or a poly(U) polymerase, as disclosed in WO 2021/018919 A1.

In some embodiments, the template-independent polymerase is a wild type or mutant pdPolθ (pdPol Theta) polymerase (pdPolθ comprises polymerase domain residues 1792-2590 of Polθ) (Hogg, et al., 2012. “Promiscuous DNA Synthesis by Human DNA Polymerase θ.” Nucleic Acids Research 40 (6): 2611-22; Malaby, et al., 2017. “Expression and Structural Analyses of Human DNA Polymerase θ (POLQ).” Methods in Enzymology 592 (May): 103-21). In a preferred embodiment, a dNTP nucleotide is tethered to pdPolθ fused to a binding agent. In one embodiment, the binding agent is fused to the pdPolθ as a fusion protein. In another embodiment, the binding agent is fused to the pdPolθ using SnoopCatcher-SnoopTag technology or the orthogonal SpyCatcher-SpyTag system (Hatlem, et al., 2019. “Catching a SPY: Using the SpyCatcher-SpyTag and Related Systems for Labeling and Localizing Bacterial Proteins.” International Journal of Molecular Sciences 20 (9)). New versions of the SpyCatcher-SpyTag system such as SpyCatcher2-SpyTag2 and SpyCatcher3-SpyTag3 demonstrate faster coupling kinetics and can also be employed for bioconjugation within the binder-writer-cNTP complex (Keeble and Howarth, 2020. “Power to the Protein: Enhancing and Combining Activities Using the Spy Toolbox.” Chemical Science 11 (28): 7281-91). In the BA-pdPolθ-dNTP binder-writer configuration, upon binding to a cognate peptide, encoding information is written via template-independent primer extension of the tethered dNTP nucleotide via the fused pdPolθ. In a preferred embodiment, the dNTP is tethered to pdPolt via a linker connecting to the terminus of the 5′ polyphosphate (triphosphate, tetraphosphate, pentaphosphate, etc.) of the nucleotide. In a 5′ triphosphate nucleotide, the linker is tethered to the terminal gamma phosphate moiety. In a preferred embodiment, the mutant pdPolθ is comprised of one or more mutants at positions P2322, A2328, L2334, E2335, Q2384, Y2387, G2388, or Y2391 (as disclosed in US 2020/0224181 A1; Randrianjatovo-Gbalou, et al., 2018. “Enzymatic Synthesis of Random Sequences of RNA and RNA Analogues by DNA Polymerase Theta Mutants for the Generation of Aptamer Libraries.” Nucleic Acids Research 46 (12): 6271-84). In a preferred embodiment, the pdPolθ-based binder-writer is used in the presence of 1-10 mM manganese cation (as disclosed in U.S. Pat. No. 10,865,396 B2, incorporated herein; Kent, et al., 2016. “Polymerase θ Is a Robust Terminal Transferase That Oscillates between Three Different Mechanisms during End-Joining.” eLife 5 (June)). In preferred embodiment, pdPolθ-based binder-writer encoding is performed in a liquid or solution containing 5 mM Mn2+, 20 mM Tris/HCl pH 8, 10% glycerol, 150 mM NaCl, 0.01% IGEPAL C6-30, 0.1 mg·ml″1 BSA (Bovine Serum Albumin) (as described in US 2020/0224181 A1, incorporated herein).

In some embodiments, the template-independent polymerase is a variant of Polθ (Pol Theta) polymerase having template-independent terminal transferase activity and disclosed in US 2018/0274001 A1, incorporated herein.

In some embodiments, it is advantageous to engineer a polymerase having template-independent terminal transferase activity specific for a particular nucleic acid moiety. In these embodiments, a set of such engineered polymerases as conjugates with binding agents can be utilized simultaneously supplemented with the specific nucleic acid moieties (e.g., nucleotide moieties). Binding agents conjugated to such engineered polymerases would compete for a component of the immobilized peptide, such as for the NTAA or modified NTAA of the peptide. Following the binding of one of the conjugates to the peptide, the engineered polymerase of the conjugate will be located in close proximity to the terminus of the nucleic acid recording tag, and will catalyze covalent addition of the specific nucleic acid moiety to the terminus of the recording tag, encoding a binding event.

In some embodiments, pdPolθ is engineered to incorporate with high specificity, in a template independent manner, a particular dNTP, such a dATP, or dCTP, or dGTP, or dTTP wherein the other three non-incorporated dNTPs are highly discriminated against. A set of four pdPolθ mutants can be engineered such that the four different dNTPS (dATP, dCTP, dGTP, and dTTP) are incorporated by their cognate pdPolθ with high specificity. Mutant residues of pdPolt in proximity to the nucleotide base (e.g., at locations 2384, 2387, and 2388) can be identified by protein engineering for enhanced base specificity. For instance, the mutant pdPolθ_{dATP} incorporates dATP with high specificity over dCTP, dGTP, and dTTP; similarly, a pdPolθ]_{dcTP} variant can be found with specificity for dCTP and so forth. In particular, a binding site for particular nucleotide bases can be engineered at proximal residues by use of Watson-Crick or Hoogsteen pseudo pairs with amino acids (see e.g., Kondo and Westhof, 2011. “Classification of Pseudo Pairs between Nucleotide Bases and Amino Acids by Analysis of Nucleotide-Protein Complexes.” Nucleic Acids Research 39 (19): 8628-37). The amino acid residues Asn, Gln, Asp, Glu, Arg and the peptide backbone (PB) are involved with binding nucleotide bases in a pseudo pairing approach. For example, adenosine bases shows preference for pseudo pairing with Asn or the peptide backbone, whereas guanine shows preference for pseudo pairing with aspartate (Kondo et al., 2011). In summary, by mutating the nucleotide base proximal residues (2384, 2387, and 2388) to Asn, Gln, Asp, Glu, and Arg, base specificity can be increased.

In some embodiments, a polymerase having template-independent terminal transferase activity is a variant of a DNA polymerase of the polX family capable of synthesizing a nucleic acid molecule without a template strand, or of a functional fragment of such a polymerase, which can incorporate a nucleic acid moiety comprising 3′-OH modification to the terminus of the nucleic acid recording tag, as disclosed in US 2020/0002690 A1, incorporated herein. The DNA polymerases disclosed in US 2020/0002690 A1 are variants of Pol IV, Pol mu, or of the terminal deoxyribonucleotidyl transferase (TdT).

In some embodiments, a polymerase having template-independent terminal transferase activity is a variant of Family A polymerase, which can incorporate a reversible modified terminator nucleic acid moiety to the terminus of the nucleic acid recording tag, as disclosed in US 2020/0370027 A1, incorporated herein.

In another embodiment of the invention, a conjugate is provided, which comprises a binding agent conjugated via a first linker to a writer enzyme, wherein said conjugate is configured to bind to a peptide comprising an associated nucleic acid recording tag joined to a solid support, said binding agent is configured to bind to said peptide, and said writer enzyme is configured to catalyze covalent addition of a nucleic acid moiety onto a terminus of said nucleic acid recording tag. Such covalently linked multi-domain molecular constructs can read amino acid information of the immobilized peptide via a binder domain and write that information into nucleic acid recording tag through a writer domain.

In some embodiments, the binder domain can be any molecular species that have some affinity for a component of the immobilized peptide, such as an N-terminal amino acid residue of the immobilized peptide. Examples of molecular species that can act as a binder domain are proteins, aptamers and other polymeric molecules that can have affinity for peptide components. The writer domain is an enzyme that can add single or oligo nucleotides (nucleic acid moieties (e.g., nucleotide moieties)) onto the 5′ or 3′ terminal of the nucleic acid recording tag (RT) associated with the immobilized peptide. Examples of writer domains are terminal deoxynucleotidyl transferase (TdT) and T4 RNA ligase.

In preferred embodiments, the binder domain (e.g., protein, aptamer etc.) is connected to the catalytically active writer domain protein (enzyme, e.g., TdT, T4 RNA ligase etc.) through a flexible linker. The linker-mediated attachment of the writer domain to the binder domain allows the binder to bind noncovalently or covalently to the immobilized peptide and localize the writer domain at the nucleic acid recording tag (RT) that is attached to the immobilized peptide. This localization increases the effective concentration of the enzyme and enables specific addition of nucleotides into the recording tag, generating an extended recording tag. The flexible linker between binder and writer domains can be a polypeptide, or any other flexible polymer made of certain number of natural or unnatural monomers. For example, the flexible polypeptide linkers can be made of many different amino acid sequences. In some embodiments, size and the flexibility of the linker will enable the binder and writer domains to interact with N-terminal amino acid residue of the immobilized peptide and with the 3′ terminus of the recording tag. In some embodiments, binder domain is a protein, and it is connected to the writer enzyme via a polypeptide linker. In other embodiments, binder domain is not a protein (e.g., an aptamer), and it is linked to the writer enzyme via a chemical linker such as polyethylene glycol.

The length of a polyethylene glycol monomer is reported to be in the range of 0.278 nm to 0.358 nm depending on the orientation of the bonds (Oestervelt, F., M. Rief, and H. E. Gaub, Single molecule force spectroscopy by AFM indicates helical structure of poly(ethylene-glycol) in water. New Journal of Physics, 1999. 1: p. 6-6). For example, the length of a PEG5000 polymer (n=113) is expected to be in the range of 31 nm to 40 nm (Sedlak, S. M., et al., Monodisperse measurement of the biotin-streptavidin interaction strength in a well-defined pulling geometry. PLOS ONE, 2017. 12(12): p. e0188722). Assuming a length of 0.36 nm per amino acid, the length of 100 amino acid residues is about 36 nm. In some embodiments, a linker equivalent of n=35 PEG monomers, 9.7 nm, can be used to link binder and writer domains of a binder-writer conjugate. In some embodiments, any linker with length of 10-12.5 nm length can be used to link binder and writer domains in the disclosed methods and compositions. For example, a polypeptide with a flexible structure made of 27 to 35 amino acid residues may be used as a linker. The amino acid composition of the linker needs be optimized. Preferably, polypeptide linker composed of Gly, Ser, Pro and Ala repeats can be used, for example, (G4S2)3, (G2APS2)3, to link the writer and binder domains of a binder-writer conjugate.

In preferred embodiments, binder and writer enzyme work coordinatively to encode structural information of a component of the immobilized peptide (peptide analyte) recognized by the binder as a nucleotide sequence. In preferred embodiments, binder and writer enzyme form a conjugate, e.g., a fusion protein, comprising a binder-writer conjugate where the binder and the writer enzyme are connected via a flexible linker. In some embodiments, the flexible linker is a polymer comprised of a polymer such as a poly alkyl chain (CH₂)ₙ, or a poly(ethylene glycol) PEG polymer chain, or a poly(ethylene oxide) PEO chain. In some embodiments, the flexible linker is a peptide linker, such as a linker that comprises amino acid sequences set forth in SEQ ID NO: 30-33.

Upon binding of the binder to the component of the immobilized peptide, the writer enzyme present in the binder-writer conjugate is located in a proximity to the terminus of the nucleic acid recording tag associated with the immobilized peptide. Then, the writer enzyme starts catalyzing addition of a nucleic acid moiety present nearby to the terminus of the recording tag, generating an extended recording tag. Therefore, specific recognition of the peptide's component by the binder is coupled with the addition of the nucleic acid moiety to the terminus of the recording tag, representing encoding process (in a single encoding cycle). Following analysis of composition of the extended recording tag (e.g., by determining incorporated nucleic acid moieties (e.g., nucleotide moieties) by using nanopore sequencing, or by full sequencing of the extended recording tag) is used to obtain information regarding specificity and, optionally, binding kinetics of one or more binders that were specifically bound to the peptide, as well as the order in which these binders were bound. Known specificities and the binding order of the binders provide identifying information regarding specific components to which the binders were bound, as well as regarding overall composition of the immobilized peptide.

In some embodiments, binders within the conjugates that specifically bind to an N-terminal amino acid (NTAA) residue, or to a modified NTAA, of the immobilized peptide are employed. In some other embodiments, binders that specifically bind to a C-terminal amino acid (CTAA) residue, or to a modified CTAA, of the immobilized peptide are employed. In these embodiments, identity of the terminal amino acid residue of the immobilized peptide can be encoded during the encoding process, and later decoded through the analysis of the extended recording tag. Terminal amino acid (TAA) residue of the immobilized peptide with a modifying reagent, thereby generating a modified TAA, before contacting the immobilized peptide with the binder-writer conjugate. Modifying reagent can be chosen to increase affinity and/or specificity of binding agents towards particular terminal amino acid residues. Examples of such modifying reagents and binding agents that can bound to TAA or modified TAA with certain levels of specificity and selectivity are disclosed in the following patent publications, incorporated herein: U.S. Pat. No. 9,435,810 B2, WO2010/065531 A1, US 2019/0145982 A1, US 2020/0348308 A1, US 2019/0145982 A1, U.S. Pat. No. 9,435,810 B2; also in U.S. patent application Ser. No. 17/539,033, and in U.S. provisional patent applications Nos. 63/133,166 and 63/250,199.

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). For example, exopeptidases (e.g., aminopeptidases, carboxypeptidases, dipeptidyl peptidase, dipeptidyl aminopeptidase), exoproteases, mutated exoproteases, mutated anticalins, mutated ClpSs, antibodies, or tRNA synthetases can be modified to create a binding agent that selectively binds to a particular NTAA. In another example, carboxypeptidases can be modified to create a binding agent that selectively binds to a particular CTAA. A binding agent can also be designed or modified, and utilized, to specifically bind a modified NTAA or modified CTAA, for example one that has a post-translational modification (e.g., phosphorylated NTAA or phosphorylated CTAA) or one that has been modified with a label (e.g., PTC, 1-fluoro-2,4-dinitrobenzene (using Sanger's reagent, DNFB), dansyl chloride (using DNS-Cl, or 1-dimethylaminonaphthalene-5-sulfonyl chloride), or using a thioacylation reagent, a thioacetylation reagent, an acetylation reagent, an amidination (guanidinylation) reagent, or a thiobenzylation reagent). Strategies for directed evolution of proteins are known in the art (e.g., Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc

In some embodiments, a binding agent can be utilized that selectively binds a modified C-terminal amino acid (CTAA). Carboxypeptidases are proteases that cleave/eliminate terminal amino acids containing a free carboxyl group. A number of carboxypeptidases exhibit amino acid preferences, e.g., carboxypeptidase B preferentially cleaves at basic amino acids, such as arginine and lysine. A carboxypeptidase can be modified to create a binding agent that selectively binds to particular amino acid. In some embodiments, the carboxypeptidase may be engineered to selectively bind both the modification moiety as well as the alpha-carbon R group of the CTAA. Thus, engineered carboxypeptidases may specifically recognize 20 different CTAAs representing the standard amino acids in the context of a C-terminal label. Control of the stepwise degradation from the C-terminus of the peptide is achieved by using engineered carboxypeptidases that are only active (e.g., binding activity or catalytic activity) in the presence of the label. In one example, the CTAA may be modified by a para-Nitroanilide or 7-amino-4-methylcoumarinyl group.

In some embodiments, the immobilized peptide is cleaved with a CTAA cleaving enzyme, such as C-terminal exopeptidases. The rate of cleavage of a C-terminal exopeptidase can be controlled to ensure cleavage of a single or few terminal amino acid residues.

For embodiments relating to CTAA binding agents, methods of cleaving CTAA from peptides or polypeptides are also known in the art. For example, U.S. Pat. No. 6,046,053 discloses a method of reacting the peptide or protein with an alkyl acid anhydride to convert the carboxy-terminal into oxazolone, liberating the C-terminal amino acid by reaction with acid and alcohol or with ester. Enzymatic cleavage of a CTAA may also be accomplished by a carboxypeptidase. Several carboxypeptidases exhibit amino acid preferences, e.g., carboxypeptidase B preferentially cleaves at basic amino acids, such as arginine and lysine. As described above, carboxypeptidases may also be modified in the same fashion as aminopeptidases to engineer carboxypeptidases that specifically bind to CTAAs having a C-terminal label. In this way, the carboxypeptidase cleaves only a single amino acid at a time from the C-terminus, and allows control of the degradation cycle. In some embodiments, the modified carboxypeptidase is non-selective as to amino acid residue identity while being selective for the C-terminal label. In other embodiments, the modified carboxypeptidase is selective for both amino acid residue identity and the C-terminal label.

In some embodiments, a C-terminal modification reagent (CTM) is configured to react with the CTAA of the polypeptide and comprises one of the following moieties: isothiocyanate, diphenylphosphoryl isothiocyanate, tetrabutylammonium isothiocyanate, sodium thiocyanate, ammonium thiocyanate, acetyl chloride, and cyanogen bromide. In some embodiments, the CTAA of the polypeptide is contacted with the CTM under conditions that allow the CTAA to be conjugated to the carboxyl reactive moiety of the CTM to form a CTM-polypeptide complex.

Engineered aminopeptidase mutants that bind to and cleave individual or small groups of labeled (biotinylated) NTAAs have been described (see, PCT Publication No. WO2010/065322, incorporated by reference in its entirety). Aminopeptidases are enzymes that cleave amino acids from the N-terminus of proteins or peptides. Natural aminopeptidases have very limited specificity, and generically eliminate N-terminal amino acids in a processive manner, cleaving one amino acid off after another (Kishor et al., 2015, Anal. Biochem. 488:6-8). However, residue specific aminopeptidases have been identified (Eriquez et al., J. Clin. Microbiol. 1980, 12:667-71; Wilce et al., 1998, Proc. Natl. Acad. Sci. USA 95:3472-3477; Liao et al., 2004, Prot. Sci. 13:1802-10).

In some embodiments, the order of the steps in the process for a degradation-based peptide or polypeptide sequencing assay can be reversed or be performed in various orders. For example, in some embodiments, the terminal amino acid labeling can be conducted before and/or after the polypeptide is bound to the binding agent.

In some embodiments, more than one encoding cycle is implemented in the disclosed methods. In these embodiments, binders that specifically bind to a TAA or to a modified TAA residue of the immobilized peptide are particularly suitable. In an exemplary embodiment, after specific recognition of the peptide's TAA by the binder, which is coupled with the addition of the nucleic acid moiety to the terminus of the recording tag, the peptide's TAA is cleaved chemically or enzymatically, exposing a newly formed TAA. Optionally, the peptide's TAA is modified before contacting with the binder to increase affinity and/or specificity for the binder. Then, the step of binding coupled with the addition of the nucleic acid moiety to the terminus of the extended recording tag is repeated, encoding structural and, optionally kinetic information, regarding the binding event and the newly formed TAA recognized by the binder are obtained. After that, the peptide's newly formed TAA is cleaved chemically or enzymatically, exposing a new TAA, and the cycle of binding/encoding and cleaving can be further repeated for one or more times, generating a further extended recording tag associated with the immobilized peptide and containing information about history of the binding events.

Exemplary methods of chemical NTAA cleavage (removal) from the immobilized peptide are disclosed in U.S. Pat. No. 9,625,469 B2, and in the following published patent applications: US 2020/0348307 A1, US 2020/0400677 A1, US 2020/0217853 A1, WO 2020/223133 A1, and in U.S. patent application Ser. No. 17/606,759.

The disclosed design can be adopted for high throughput peptide analysis or sequencing, and permits use of moderate affinity, relatively non-selective binders, which recognize, for example, a group of terminal amino acids instead of a single TAA. In some embodiments, a set of binders is selected that altogether covers specificity for all 20 terminally located, standard or natural amino acid residues. In some embodiments, each of the selected binders may bind more than one amino acid that is in the N-terminal position of the immobilized peptide. A set of moderate affinity, relatively non-selective binders can be used to determine identity of the immobilized peptide, which can be derived based on the collected structural and, optionally, kinetic information from multiple TAA recognition events due to existing redundancy.

The data obtained from nucleotide sequence of the extended recording tag associated with the immobilized peptide may provide ambiguous information regarding each of the amino acid residues of the peptide analyte, creating a pattern of amino acid options at certain places in the sequence of the peptide. For example, if a first NTAA binder is specific for negatively charged NTAA residues (such binders are disclosed, for example, in U.S. provisional patent application No. 63/250,199), the proposed NTAA residue of the peptide would be D/E. Next, if a second NTAA binder has specificity towards small hydrophobic NTAA residues, the proposed NTAA residue of the peptide would be G/A/V/I/L. A third NTAA binder will have an independent specificity, and so on. The obtained pattern of amino acid options can be then searched against known proteome sequences in order to identify the immobilized peptide. In some embodiments, the peptide will be identified by comparison of the generated pattern with other patterns generated computationally using a database of possible protein sequences from the organism being analyzed (e.g., if a human sample is analyzed, then a human proteome database is used to generate theoretical patterns for comparison). If a sample potentially contains a proteomic mixture of different species, then their proteomes can be combined before extracting theoretical peptide patterns for comparison. In other embodiments, genomic databases can be utilized to extract theoretical peptide patterns from coding regions of the genome(s).

Computer simulations have shown that relatively simple labeling schemes of specific amino acid residues are sufficient to identify most proteins in the human proteome. For example, employing only 1 to 4 amino acid specific fluorescent labels can yield patterns capable of uniquely identifying at least one peptide from most of the known human proteins (See Swaminathan J, Boulgakov A A, Marcotte E M. A theoretical justification for single molecule peptide sequencing. PLoS Comput Biol. 2015 Feb. 25; 11(2):e1004080). Increasing the number of distinct label types improves identification up to 80% within only 20 experimental cycles (See Swaminathan J, Boulgakov A A, Marcotte E M. PLoS Comput Biol. 2015 Feb. 25; 11(2):e1004080). Thus, based on these calculations, collecting pattern information about 5-6 amino acid residues using non-selective binders will be enough or sufficient for identifying the peptide from a proteome, e.g., the human proteome, with certain probability.

In some embodiments, during the analyzing step, an artificial intelligence (AI) model, e.g., an AI model employing probabilistic neural networks (PNN), can be applied to calculate probabilities of occurrence of specific types of amino acid residues in corresponding places in amino acid sequence of the peptide based on a nucleotide sequence of the (further) extended recording tag.

In some embodiments, a set of relatively specific NTAA binders may be utilized in in the disclosed conjugates (as attached to a writer enzyme). Examples of such NTAA binders are disclosed, for example, in U.S. Pat. Nos. 9,435,810 B2 and 10,852,305 B2, incorporated by reference herein.

In some embodiments, the disclosed method further comprises, before the first contacting step, modifying an N-terminal amino acid (NTAA) residue of the peptide with a modifying reagent, thereby generating a modified NTAA residue of the peptide.

In some embodiments, binders are used that have increased binding kinetics (e.g., decreased dissociation rate) to specific modified NTAA residues. In some embodiments, binders are used that have particular binding affinities to specific modified NTAA residues. In some embodiments, engineered binders specific for modified NTAA residues and use thereof in the methods disclosed herein can be derived from natural lipocalin scaffolds, as disclosed in U.S. patent application Ser. No. 17/539,033, filed on Nov. 30, 2021. In some preferred embodiments, an engineered binder that specifically binds to an N-terminally modified target polypeptide modified by an N-terminal modifier agent is used, wherein:
(i) the N-terminally modified target polypeptide has a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide;
(ii) the engineered binder specifically binds to the N-terminally modified target polypeptide through interaction between the engineered binder and the M-P1 of the N-terminally modified target polypeptide; and (iii) the engineered binder comprises an amino acid sequence having at least about 80% sequence identity to the amino acid sequence selected from the group consisting of SEQ ID NO: 17-SEQ ID NO: 19. In some other preferred embodiments, the engineered binder comprises an amino acid sequence having at least about 80% or 90% sequence identity to the amino acid sequence selected from the group consisting of SEQ ID NO: 17-SEQ ID NO: 19, or other engineered binder specific to particular modified NTAA residues, as disclosed in U.S. patent application Ser. No. 17/539,033.

In some embodiments, a set of engineered binders specific for modified NTAA residues and conjugated to a writer enzyme is used in the methods disclosed herein. Such binders may have different affinities towards different modified NTAA residues. In some preferred embodiments, a set of engineered binders specific for modified NTAA residues is used, the set comprising at least two engineered binders, wherein:
each engineered binder from the set of engineered binders is configured to specifically bind to an N-terminally modified target polypeptide modified with an N-terminal modifier agent and having a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide;
each engineered binder from the set of engineered binders is configured to specifically bind to the N-terminally modified target polypeptide through interaction between the engineered binder and the modified NTAA residue of the N-terminally modified target polypeptide, wherein engineered binders from the set of engineered binders are configured to specifically bind to different modified NTAA residues of target polypeptides modified with the same or different N-terminal modifier agents; and
at least one engineered binder from the set of engineered binders comprises an amino acid sequence having at least about 80% or 90% sequence identity to the amino acid sequence selected from the group consisting of SEQ ID NO: 17-SEQ ID NO: 19. In some embodiments, multiple or all of the engineered binder from the set of engineered binders comprise an amino acid sequence having at least about 80% or 90% sequence identity to the amino acid sequence selected from the group consisting of SEQ ID NO: 17-SEQ ID NO: 19, as disclosed in U.S. patent application Ser. No. 17/539,033.

In some preferred embodiments, the N-terminal modifier agent that is used to modify modified target polypeptide is selected from the group consisting of compounds of the following formula:

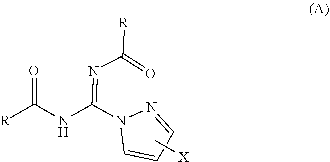

wherein R is CH₃, CF₃, OC(CH₃)₃, or OCH₂C₆H₅,
and X is H, CH₃, CF₃, CF₂H, or OCH₃;

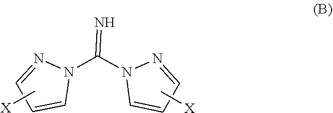

wherein X is H, CH₃, CF₃, CF₂H, OCH₃, or SO₂NH₂;

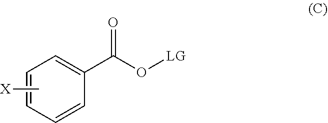

wherein X is H, F, Cl, OCH₃, OCF₃, CN, or SO₂NH₂,
and LG is succinimide, pentafluorophenyl, or tetrafluorophenyl;
and

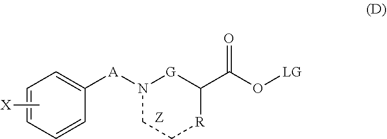

wherein X is H, F, Cl, NH₂, OCH₃, OCF₃, CN, or SO₂NH₂, A=CONH or SO₂, G=0 or 1 CH₂, R is any amino acid or unnatural amino acid, and Z ring=0 (not there), 1, 2, or 3 CH₂.

In some embodiments, engineered binders specific for modified NTAA residues and use thereof in the methods disclosed herein can be derived from natural metalloprotein scaffolds, as disclosed in U.S. provisional patent application No. 63/250,199, filed on Sep. 29, 2021, and in the PCT application No. PCT/US2021/065798, filed on Dec. 30, 2021. In some preferred embodiments, an engineered metalloprotein binder that specifically binds to an N-terminally modified target peptide modified by an N-terminal modifier agent is used, wherein: a) the N-terminally modified target peptide has a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
b) the engineered metalloprotein binder specifically binds to the N-terminally modified target peptide through interaction between the engineered metalloprotein binder and the Z-P1 of the N-terminally modified target peptide; and
c) the engineered metalloprotein binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less.

In some preferred embodiments, the engineered metalloprotein binder comprises an amino acid sequence having at least about 80% or 90% sequence homology to any one of the amino acid sequences selected from the group consisting of SEQ ID NO: 20-SEQ ID NO: 22.

In some embodiments, the engineered binder binds to the N-terminally modified target peptide with a thermodynamic dissociation constant (Kd) of 200 nM or less. In some preferred embodiments, the engineered binder binds to the N-terminally modified target peptide with a thermodynamic dissociation constant (Kd) of 100 nM or less.

In some embodiments, multicycle encoding methods described herein utilize a step of modifying (functionalizing) an N-terminal amino acid (NTAA) residue of the peptide with a modifying reagent before contacting the peptide with binder-writer conjugates, thereby generating a modified NTAA residue of the peptide, and, after the encoding step, cleaving the modified NTAA residue of the peptide.

In some embodiments, the modification of the NTAA residue of the immobilized peptide and the cleaving of the modified NTAA residue are performed according to methods disclosed in the published patent publication No. WO 2020/223133 A1, and in U.S. patent application Ser. No. 17/606,759. Scheme I shows an exemplary functionalization of the peptide's NTAA residue to form compounds of Formula (II), followed by inducing elimination of the functionalized NTAA under mild conditions at around pH 5-10.

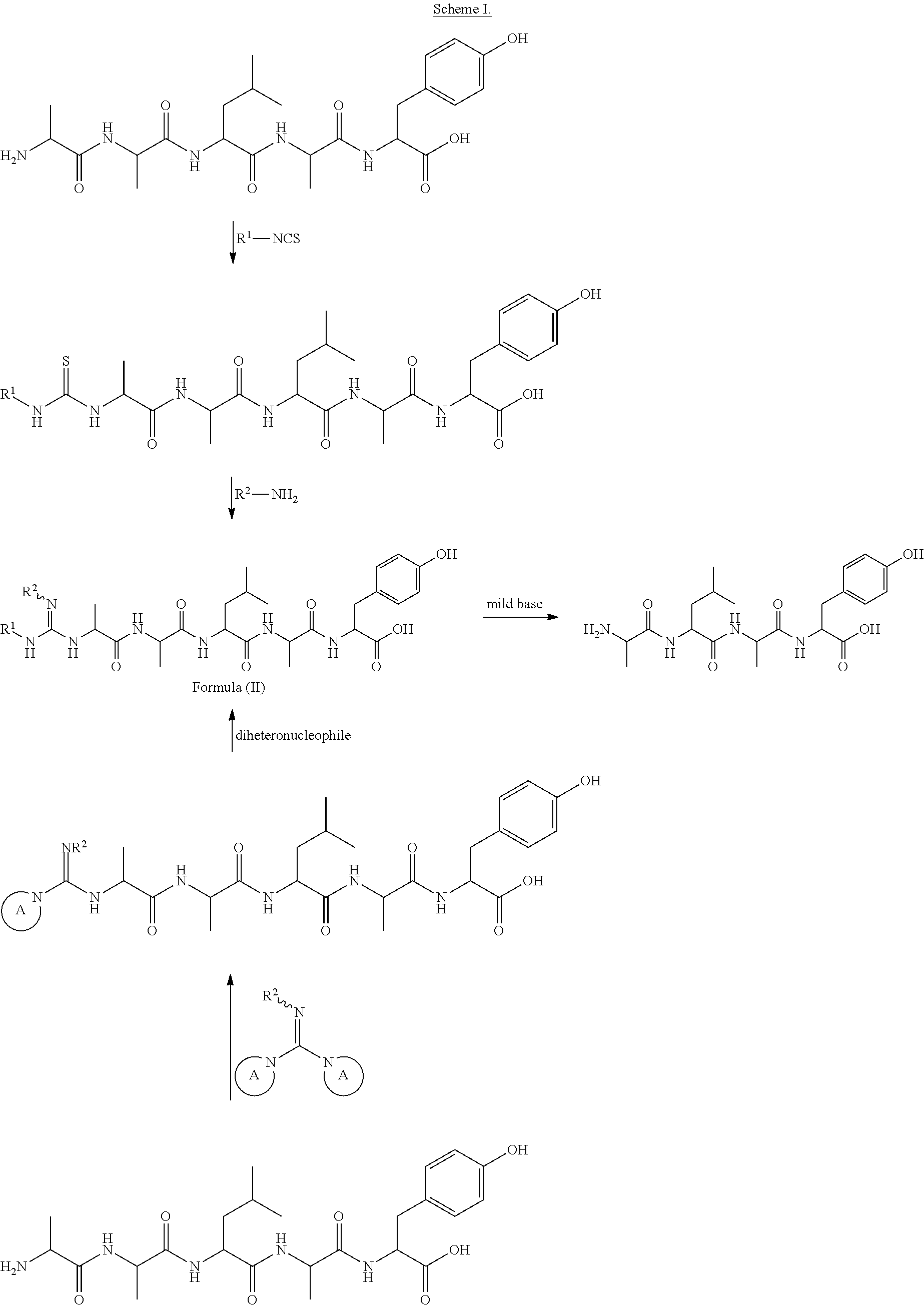

The reactions shown in Scheme I result in cleavage of the NTAA from a peptide under mild conditions, and thus enable a method for removal of the NTAA from a peptide. The described method can be used repeatedly, to remove one NTAA at a time from the immobilized peptide. The mild reaction conditions involved make it possible to perform these reactions in the presence of acid-sensitive moieties, such as nucleic acid recording tags. The nucleic acids are stable to the conditions used for functionalization and cleavage of the NTAA of a peptide as shown by data presented in the published patent application WO 2020/223133 A1, and in U.S. patent application Ser. No. 17/606,759.

In some embodiments, functionalization of the NTAA using a chemical reagent comprising a compound of Formula (AA) and the subsequent elimination are as depicted in the following scheme:

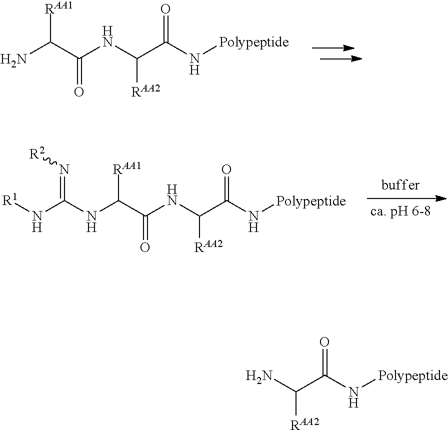

wherein R¹ and R² are as defined above and R^{AA1} is the side chain of the NTAA of a peptide.

In some embodiments, the functionalized NTAA is removed by a suitable reagent. The mixture is typically maintained at 25° C.-100° C. for 10-60 minutes in the medium to effect removal of the NTAA. An example of a suitable medium is water with phosphate, sodium chloride, tween 20 (surfactant) at pH 5-10, and is heated at 25° C.-60° C. for 1 to 60 minutes containing a suitable reagent such as a diheteronucleophile. In some embodiments, the elimination is performed using an aqueous formulation that includes 0.1M to 2.0M sodium, potassium, cesium, or ammonium phosphate buffer or sodium, potassium, or ammonium carbonate buffer at a pH 5.5-9.5 at 50-100° C. for 5-60 minutes. In some embodiments, the suitable reagent for NTAA elimination comprises a hydroxide, ammonia, or a diheteronucleophile, typically at a concentration of 0.15 M-4.5 M.

In other embodiments, cleaving the modified NTAA residue of the peptide can be achieved by methods disclosed in US 2020/0348307 A1, incorporated herein.

In yet other embodiments, cleaving the modified NTAA residue of the peptide is achieved by using an engineered enzyme, such as an engineered dipeptidyl aminopeptidase disclosed in the published patent applications US 2021/0214701 A1 and WO 2021/141924 A1, incorporated herein.

In preferred embodiments of the disclosed methods, cleaving the modified NTAA residue of the peptide is done by an engineered enzyme, such as a modified cleavase, which is configured to cleave a peptide bond between a terminally labeled amino acid residue and a penultimate terminal amino acid residue of a polypeptide, wherein the modified cleavase is derived from a dipeptidyl aminopeptidase, which removes an unlabeled terminal dipeptide from a polypeptide, wherein the dipeptide aminopeptidase comprises an amino acid sequence having at least 20% sequence identity to the amino acid sequence of SEQ ID NO: 23 and also comprising an asparagine residue at a position corresponding to position 191 of SEQ ID NO: 23, a tryptophan residue or phenylalanine residue at a position corresponding to position 192 of SEQ ID NO: 23, an arginine residue at a position corresponding to position 196 of SEQ ID NO: 23, an asparagine residue at a position corresponding to position 306 of SEQ ID NO: 23, and an aspartate residue at a position corresponding to position 650 of SEQ ID NO: 23; and wherein the modified cleavase comprises two or more amino acid substitutions in the residues corresponding to positions N191, W/F192, R196, N306, and D650 of SEQ ID NO: 23, as disclosed in the patent application US 2021/0214701 A1. In some other preferred embodiments, a modified cleavase is used comprising an amino acid sequence that is at least 80% identical to the amino acid sequence set forth in SEQ ID NO: 24, as disclosed in the U.S. Pat. No. 11,427,814 B2, incorporated by reference herein.

In some other preferred embodiments, a set of modified cleavases, comprising at least two different modified cleavases, is used to cleave the modified NTAA residue of the peptide, wherein:
(i) each of the modified cleavases from the set of modified cleavases is configured to cleave a peptide bond between a terminally labeled amino acid residue and a penultimate terminal amino acid residue of a polypeptide, wherein the modified cleavase is derived from a dipeptidyl aminopeptidase, which removes an unlabeled terminal dipeptide from a polypeptide, wherein the dipeptide aminopeptidase comprises an amino acid sequence having at least 20% sequence identity to the amino acid sequence of SEQ ID NO: 23 and also comprising an asparagine residue at a position corresponding to position 191 of SEQ ID NO: 23, a tryptophan residue or phenylalanine residue at a position corresponding to position 192 of SEQ ID NO: 23, an arginine residue at a position corresponding to position 196 of SEQ ID NO: 23, an asparagine residue at a position corresponding to position 306 of SEQ ID NO: 23, and an aspartate residue at a position corresponding to position 650 of SEQ ID NO: 23; and wherein the modified cleavase comprises two or more amino acid substitutions in the residues corresponding to positions N191, W/F192, R196, N306, and D650 of SEQ ID NO: 23; and
(ii) the modified cleavases from the set of modified cleavases have different specificities for terminally labeled amino acids, which the modified cleavases are configured to remove, as disclosed in the U.S. Pat. No. 11,427,814 B2, incorporated by reference herein.

In preferred embodiments, the modified cleavase does not remove an unlabeled terminal dipeptide from the polypeptide.

In preferred embodiments, the modified cleavase comprises at least three amino acid substitutions in the residues corresponding to positions N191, W/F192, R196, N306, and D650 of SEQ ID NO: 23.

Multiple embodiments can be implemented for specific arrangement and type of the nucleic acid moiety used during the encoding process and representing the encoded structural information.

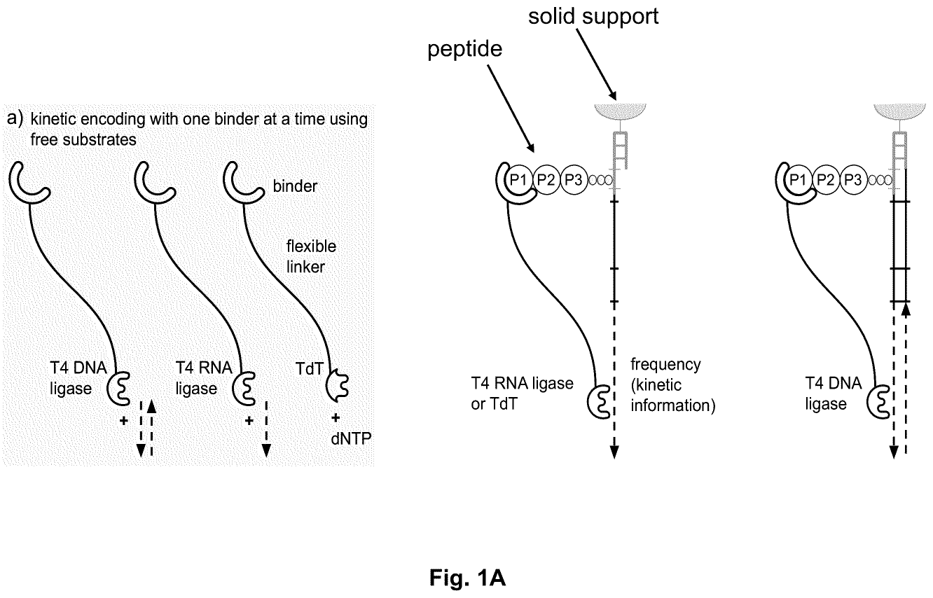

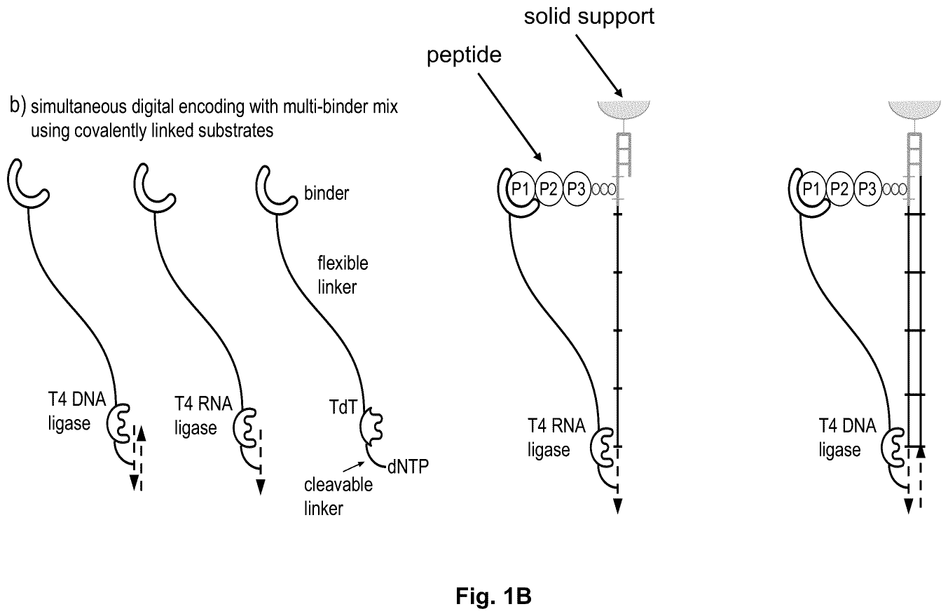

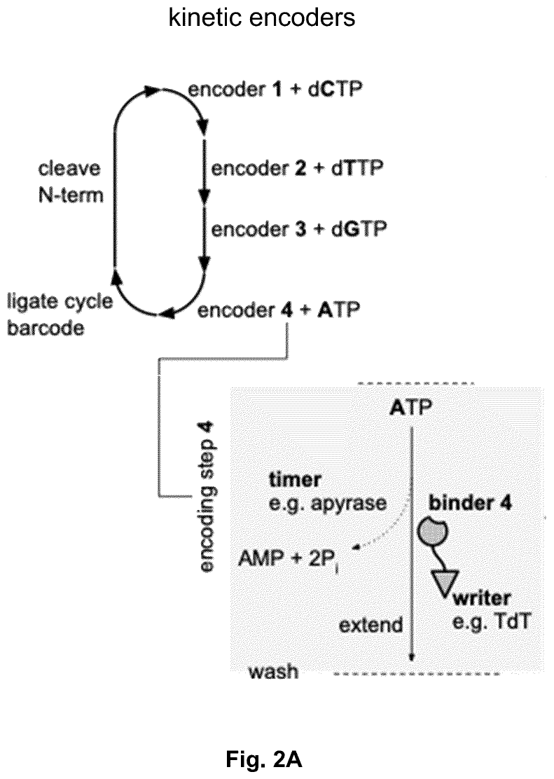

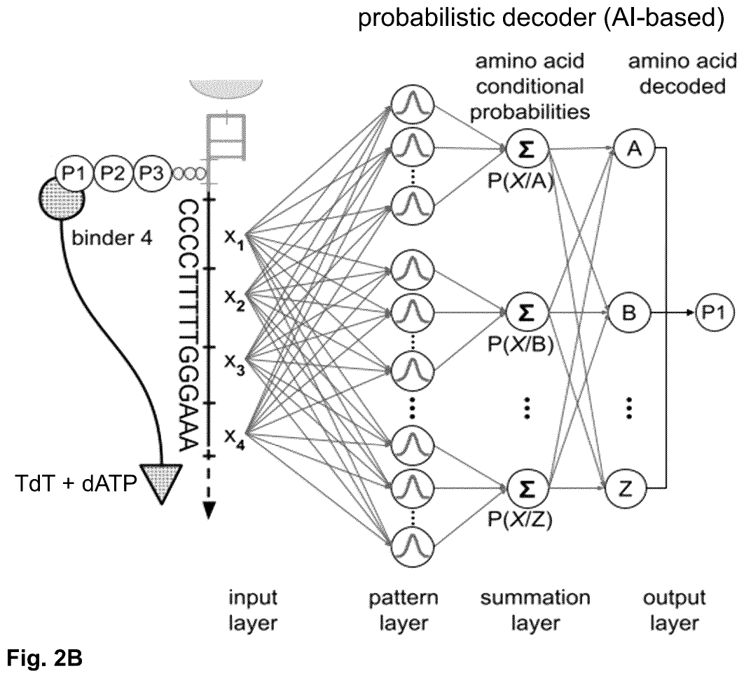

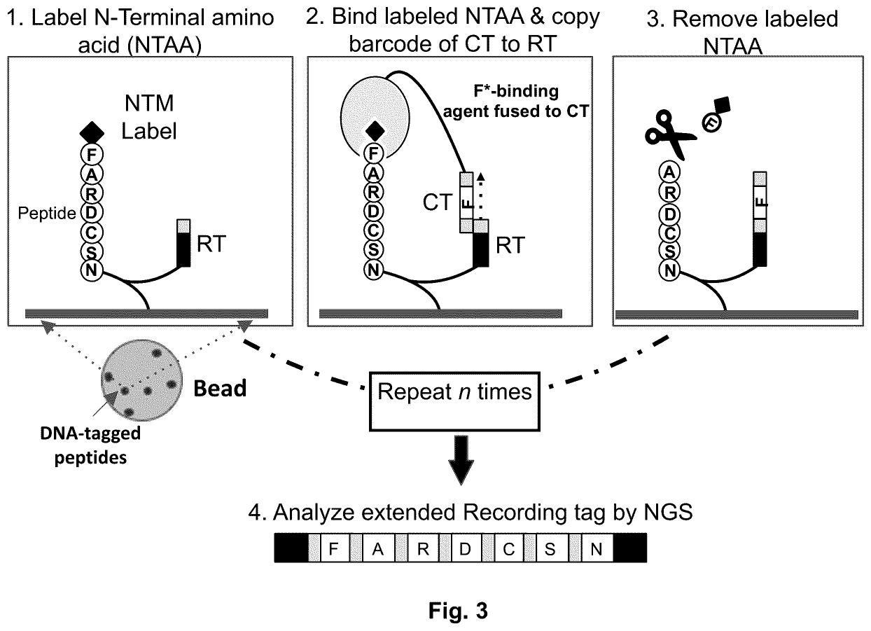

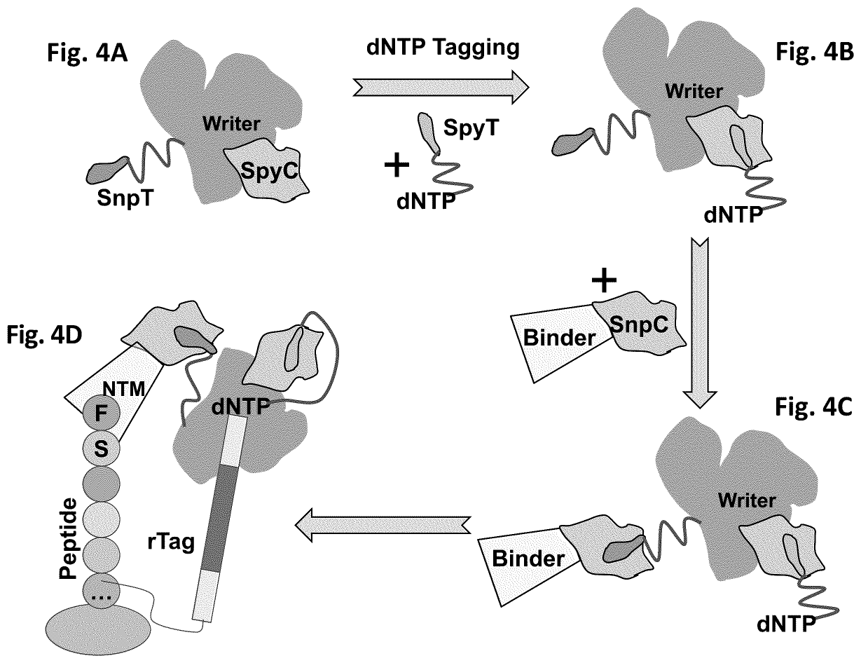

In some embodiments, encoding of peptide's structural information into nucleic acid sequence added to the terminus of the nucleic acid recording tag occurs using one binder-writer conjugate at a time together with a free single substrate (a single nucleic acid moiety) (see, e.g., FIG. 1A and FIG. 2A). In these embodiments, the binders that recognize different components of the peptide analyte (such as different NTAA or modified NTAA residues of the peptide analyte) are added sequentially.

In other embodiments, a few or several (2, 3, 4, 5 or more) binder-writer conjugates are used simultaneously (see, e.g., FIG. 1B). In some of these embodiments, each binder-writer conjugate is tethered with a specific nucleic acid moiety in such a way that the writer enzyme of this binder-writer conjugate can incorporate to the terminus of the nucleic acid recording tag only this specific nucleic acid moiety. Accordingly, by establishing identities of the incorporated nucleic acid moieties (e.g., nucleotide moieties), one can obtain information regarding binding history of the peptide analyte, and decode binding specificities (and in some cases binding kinetics) of the binders that bound the peptide analyte. Binding kinetics of the binders can be identified if more than one nucleic acid moieties (e.g., nucleotide moieties) can be incorporated during the binding event. In preferred embodiments, incorporation rate is proportional to, or at least positively correlates with, binding affinity of the binder for the NTAA or modified NTAA residues of the peptide analyte.

60) In some embodiments, binder-writer encoding is performed using dNTP-labeled binders in combination with a solution-phase template independent polymerase writer(s) such as Polθ or TdT. Upon binding, the solution-phase writer uses the proximal tethered nucleotide to label the 3′ end of the associated recording tag. By controlling the concentration of binder-dNTP and writer enzyme, the signal to noise can be optimized. Additionally, using phosphate-tethered dNTPs enables multiple writing events since each incorporated nucleotide harbors a free 3′ OH for further extension. Physically separating the binder-dNTP and the writer allows greater control of the kinetics of encoding since the concentration of the binder-dNTP can be adjusted independently from the writer concentration, at the same time a particular binder can be associated with a particular dNTP enabling encoding of the binder identity.

In a preferred embodiment, the length of the encoding region is controlled by spiking in fixed ratio of reversible terminators in lieu of the dNTP. In this way, a particular binder-writing incubation may produce, on average 5 nucleotide additions of a particular dNTP with the fifth nucleotide incorporation comprised of the reversible terminator. After the completion of the binder/writer step, the termination is reversed. An exemplar reversible terminator is the 3′-O-azidomethyl-dNTPs used in Illumina NGS sequencing with termination reversed by incubation with TCEP. Other reversible terminators include 3′O-allyl terminated and 3′O-amino terminated nucleotides which can be 3′ cleaved with palladium complexes and sodium nitrite respectively. Engineered polymerases such as engineered TdT and engineered Polθ are particularly adept at using reversible terminator nucleotides in template independent primer extension reactions. (US 2020/0370027A1, WO 2020/161480 A1, US 2019/0078065 A1). Exemplary sequences of TdT and Polθ enzyme useful for methods disclosed herein are indicated in SEQ ID NO: 10 and 11.

In some embodiments, the writer is a modified terminal dideoxynucleotide transferase (TdT) enzyme possessing mutations as described in WO 2020/161480 A1. Namely, a TdT from Lepisosteus oculatus (Spotted Gar) (SEQ ID NO: 12) or a homologue with any of the following mutations: M183R, Q300D, or D488P in either the full length or truncated version (see SEQ ID NO: 13-15).

In some embodiments, the binder-writer is generated as a fusion protein during expression, and in other embodiments, the binder-writer complex is generated by covalently coupling the binder to the writer post expression/purification of the individual proteins. In a preferred embodiment, post-expression bioconjugation can be accomplished using the SpyCatcher-SpyTag system or the orthogonal SnoopCatcher-SnoopTag system or equivalent isopeptide split protein systems (See e.g., Hatlem, et al., 2019). In one embodiment, the SpyCatcher is positioned near the N or C-terminus of the binder, and the SpyTag is positioned near the N or C-terminus (or internal) of the writer enzyme, or vice versa. A simple incubation of the binder-SpyCatcher fusion with the SpyTag-writer fusion generates the fused binder-writer construct.

DNA pol theta, a unique A-type polymerase, and DNA Pol mu, an X-type polymerase in the same family as TdT, can be engineered to efficiently incorporate 3′-O modified reversible terminators in a template-independent fashion as described by US 2019/0078065 A1 and herein incorporated by reference in its entirety. In some embodiments, the one or more mutations in the modified DNA Pol theta polymerase family, comprising homologs, orthologs, or paralogs thereof, can be an insertion of a sequence comprising ESTFEKLRLPSRKVDALDHF (SEQ ID NO:8) into a loop 1 region of human Pol theta family. For example, SEQ ID NO:8 can be inserted into or substituted with amino acids at positions 2071-2080 of human DNA polymerase theta. In still other embodiments, the one or more mutations in the modified Pol theta family can be within a finger loop adjacent to nucleotide binding site (NBS) motif located at positions 1990-1995 of human DNA polymerase theta. In other embodiments, the one or more mutations in the modified DNA Pol theta polymerase family can be within a finger to palm NBS motif located at positions 2019-2032 of human DNA polymerase theta. In still other embodiments, the one or more mutations in the modified DNA Pol theta polymerase can be within a Loop1 flanking region motif located at positions 2081-2085 of human DNA polymerase theta. In further embodiments, the one or more mutations in the modified DNA Pol theta polymerase can be within a Loop1 flanking in palm motif located at positions 2105-2113 of human DNA polymerase theta. In yet other embodiments, the one or more mutations in the modified DNA Pol theta polymerase can be within a palm NBS motif located at positions 2121-2192 of human DNA polymerase theta. In alternate embodiments, the one or more mutations in the modified DNA Pol theta polymerase family can be within a palm NBS flanking region motif located at positions 2195-2200 of human DNA polymerase theta.

In some embodiments, it is advantageous to reduce formation of a secondary structure at the terminus of the nucleic acid recording tag, which increase efficiency of nucleotide addition by the template-independent polymerase (which is the writer enzyme) conjugated with a binder. In some embodiments, secondary structure formation of the recording tag is prevented or reduced by using pseudo-complementary (PC) nucleotides as substrates for the writer enzyme, so the nucleic acid recording tag will be extended with the pseudo-complementary nucleotides. Pseudo-complementary (PC) nucleotides contain base analogs that form weak base pairs with one another, but form strong base pairs with standard bases (see, e.g., U.S. Pat. No. 5,912,340 A, incorporated herein). The strength of base pairing in PC nucleotides can be determined by standard means in the art, such as by measuring the melting temperature. PC nucleotides include modified bases (e.g., A′) of such nature that the modified base forms stable hydrogen bonded base pairs (e.g., A′=T) with the natural partner base (e.g., T), but does not form stable hydrogen bonded base pairs with its modified partner (e.g., A′−/−T′). This may be accomplished when in a hybridized structure the modified base A′ is capable of forming two or more hydrogen bonds with its natural complementary base T, but only one or no hydrogen bonds with its modified partner (T′). Thus, the matched pair of nucleotides do not form substantially stable hydrogen bonded hybrids with one another, as manifested in a melting temperature (under physiological or substantially physiological conditions) of approximately 40° C. or less.

In preferred embodiments, the nucleotide duplex formed of a pair of PC nucleotides has a melting temperature under physiological conditions of less than approximately 40° C.

In preferred embodiments, PC polynucleotides have diminished intramolecular and intermolecular secondary structures. The use of PC nucleotides in writing on the recording tag minimizes the formation of secondary structures in the extended recording tag, which can increase efficiency of the writer enzyme.

Exemplar pseudo-complementary nucleotides include 2-aminoadenine (nA) and 2-thiothymine (sT) which pair with native T and A respectively, but nA and sT don't pair efficiently with each other (Lahoud, et al., 2008. “Properties of Pseudo-Complementary DNA Substituted with Weakly Pairing Analogs of Guanine or Cytosine.” Nucleic Acids Research 36 (22): 6999-7008; Lahoud, et al., 2008. “Enzymatic Synthesis of Structure-Free DNA with Pseudo-Complementary Properties.” Nucleic Acids Research 36 (10): 3409-19). Compared with the Watson-Crick base pair between adenine (A) and thymine (T), the pair between nA and sT is unstable because of the steric clash between the exocyclic amine of nA and the large size of the sulphur atom of sT. While the nA:sT base pair is unstable, the base pairing strength of the A:sT pair is similar to that of an A:T base pair. Other examples include 7-methyl-7-deazaguanine (MecG) or 6-hydroxypurine (hypoxanthine) and N4-ethylcytosine (EtC) which base pair with native C and G respectively, but not each other (Hoshika, et al., 2010. “Artificial Genetic Systems: Self-Avoiding DNA in PCR and Multiplexed PCR.” Angewandte Chemie 49 (32): 5554-57). Select polymerase enzymes such as 9°N polymerase can efficiently transcribe a pseudo-complementary strand into a native strand (Lahoud et al., 2008).

In another embodiment, two, three or more nucleotides are simultaneously tethered to a binder-writer complex. The combination of nucleotides tethered to a particular binder-writer can be used as a code that is installed into the recording tag and used for determining the identity of the binder from the extended recording tag sequence. A binder-writer configuration composed of a binding agent fused to a template-independent polymerase labeled with two tethered nucleotides enables expansion of coding possibilities. By using two nucleotide labels, both of which can be incorporated onto the proximal recording tag upon binding enables a set of 10 codes to be generated, namely {A/C, A/G, A/T, C/G, C/T, G/T, A/A, C/C, G/G, and T/T}. For accurate encoding, the residence time of the binder should be much greater than the turnover time (1/kcat) of the polymerase enzyme. If a particular cognate binder is encoded by {C/T}, multiple kinetic on-off binding events may be recorded as CTTCCTCTTCC (SEQ ID NO: 25) or other alternatively composed C/T strings.

In another embodiment, the binder-writer-nucleotide complex is comprised of multiple tethered nucleotides configured such that the tethered nucleotides are written sequentially to the recording tag from a single binder-writer-nucleotide complex. Moreover, the tethered nucleotides can be designed to have different rates of incorporation such that the order of incorporation creates the code for a particular binder. In this way, a set of 3 tethered nucleotides can generate 4³=64 different trimer codes. This size coding space allows some limited error correction to be employed and also provide several codons to clock the cleavage cycle in the ProteoCode™ assay. An exemplar method of generating different nucleotide incorporation rates is to employ tethered nucleotides with a differing number of 5′ phosphate moieties (Sood, et al., 2005. “Terminal Phosphate-Labeled Nucleotides with Improved Substrate Properties for Homogeneous Nucleic Acid Assays.” Journal of the American Chemical Society 127 (8): 2394-95). The first incorporation can employ a pentaphosphate nucleotide (tethered via epsilon phosphate) which couples the fastest, the second incorporation can employ a tetraphosphate nucleotide (tethered via delta phosphate) which couples fast, and the final incorporation can employ a triphosphate nucleotide (tethered via gamma phosphate) which couples the slowest. Additionally, the rates of incorporation can be adjusted using alpha phosphate backbone modifications (e.g., α-thiophosphate or α-boronophosphate), base modifications, and ribose sugar modifications (Dellafiore, et al., 2016. “Modified Nucleoside Triphosphates for In-Vitro Selection Techniques.” Frontiers in Chemistry 4 (May): 18).

In some embodiments, reversible terminators can be employed as the tethered nucleotides wherein the different tethered nucleotides, or at least a subset have differing reversible terminator chemistries.

In some embodiments, the binder or writer-tethered reversible terminators are base-labeled with an oligonucleotide barcode as described by Baccaro et al. (Baccaro, et al., 2012. “Barcoded Nucleotides.” Angewandte Chemie 51 (1): 254-57). In a preferred embodiment, the identity of the binder or binder-writer complex can be uniquely determined by the barcode sequence. In one embodiment, the barcode oligonucleotide is ligated to the reversible terminator after incorporation into the proximal recording tag and removal of the 3′ blocking group. In a preferred embodiment, the oligonucleotide barcode is 2-8 bases in length. In one embodiment, the oligonucleotide barcode is tethered to the nucleotide via an alkyl, PEG, or PEO linker with 2-18 chain lengths. In one embodiment, the oligonucleotide barcode is tethered to the base via a C5 position for pyrimidines or C7 position for 7-deaza-purines. In one embodiment, the tether is comprised of a cleavable linkage. In a preferred embodiment, the cleavable linkage generates a free 3′ terminus. In one embodiment, the oligo barcode is tethered via it 3′ end to enable ligation of the free (5′ phosphate) end to the nascent 3′ OH of the deblocked incorporated nucleotide. In one embodiment, the oligonucleotide barcode is ligated using a single stranded DNA/RNA ligase such as TS2126 Rnl1 (e.g., Circligase) (Blondal, et al., 2005. “Isolation and Characterization of a Thermostable RNA Ligase 1 from a Thermus Scotoductus Bacteriophage TS2126 with Good Single-Stranded DNA Ligation Properties.” Nucleic Acids Research 33 (1): 135-42). In another embodiment, the oligo barcode is tethered via it 3′ end to enable chemical “CuAAC click” ligation of a free 5′-azide group to the nascent 3′ propargyl group of the terminated nucleotides to generate an inter-nucleotide triazole linkage (El-Sagheer, et al., 2011. “Biocompatible Artificial DNA Linker That Is Read through by DNA Polymerases and Is Functional in Escherichia Coli.” Proceedings of the National Academy of Sciences 108 (28): 11338-43). In a preferred embodiment, the ligated oligonucleotide barcode is cleaved from its original base tether to generate a free 3′ end.

In some embodiments, the first composition and/or the second composition comprise a nucleotide triphosphate or another nucleic acid moiety covalently tethered to the template-independent polymerase, e.g., the DNA ligase, or the RNA ligase, via a linker comprising a selectively cleavable linkage, such as linkage that can be cleaved without cleaving other linkages in components of the system (such as in conjugates, solid support, etc.). In some embodiments, the linker comprises alkyl, PEG, or PEO moiety with 2-18 chain lengths. In a preferred embodiment, the cleavable linkage generates a free 3′ terminus of the incorporated nucleic acid moiety on the recording tag.

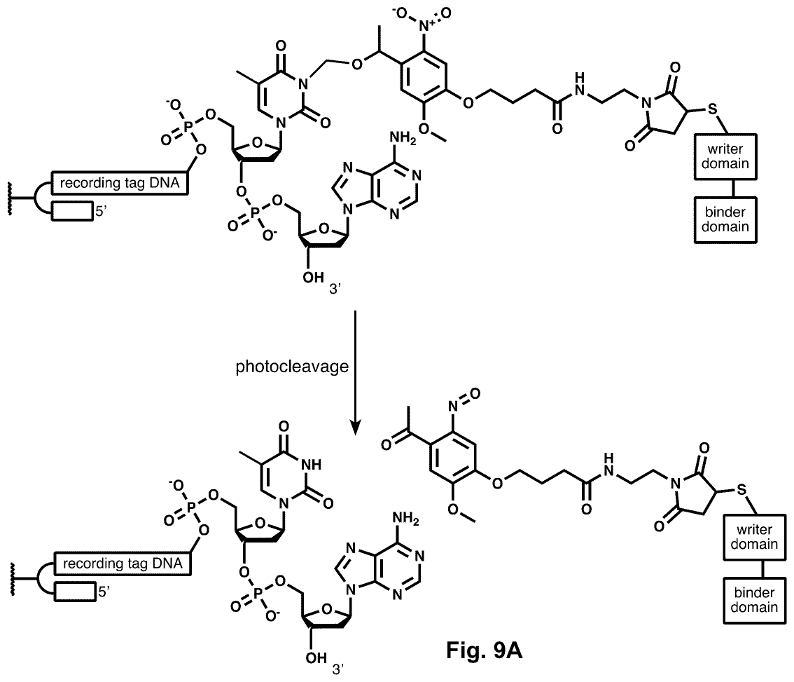

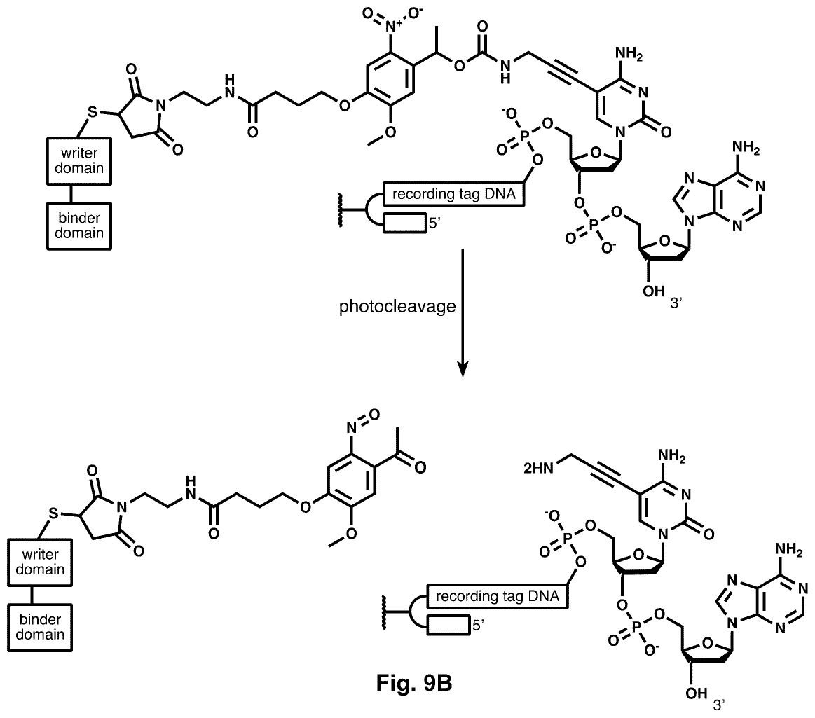

In some embodiments, a functional binder-writer-nucleic acid moiety conjugate is provided, where the nucleic acid moiety (e.g., dNTP) is attached to the writer enzyme (e.g., TdT) via a linker comprising a selectively cleavable linkage. The linker-mediated attachment of the writer enzyme to the nucleic acid moiety allows the nucleic acid moiety to bind noncovalently at the active site of the writer domain. Nucleic acid moieties (e.g., nucleotide moieties) may be covalently attached to the writer enzyme using commercially available linkers. The linker-mediated attachment of the nucleic acid moiety can occur at any position of the writer enzyme and nucleic acid moiety that allows the writer domain to incorporate the nucleic acid moiety into proximal recording tag. For example, the attachment of linker can occur on the 5′ gamma phosphate of the nucleotide (see, e.g., U.S. Pat. No. 10,767,221 B2). In another embodiment, the linker is tethered to the base via a C5 position for pyrimidines or C7 position for 7-deaza-purines. Exemplary structures of binder-writer-nucleic acid moiety conjugates are shown in FIG. 9A-9B.

In some embodiments of the disclosed methods and compositions, the linkers for writer-nucleic acid moiety conjugates are used as disclosed in U.S. Pat. No. 11,254,961 B2, incorporated herein.

A variety of protein engineering methods can be utilized to make functional binder-writer-nucleic acid moiety conjugates; some of these methods are described in Examples below. In one embodiment, a cysteine handle in the writer enzyme can be utilized and configured to form a covalent bond with a nucleic acid moiety. In one particular embodiment, the writer enzyme is TdT (PDB ID: 4I27); some solvent-exposed cysteines are mutated (Cys188Ala, Cys216Ser, Cys378Ala, and Cys438Ser), and the cysteine handle (Cys302) is used to react with maleimide. In another particular embodiment, the writer enzyme is T4 RNA ligase 1 (Rnl1) (PDB ID: 2C5U); solvent-exposed cysteines are mutated (Cys13Ala, Cys315Ala, Cys357Ala), and the cysteine handle (Ser125Cys) is introduced to react with maleimide.

In some embodiments, during encoding process, a nucleic acid moiety covalently tethered to the template-independent polymerase is incorporated into growing extended recording tag; after incorporation, a selectively cleavable linkage between the nucleic acid moiety and the template-independent polymerase is cleaved, releasing the template-independent polymerase. Exemplary cleavage chemistries are shown in FIG. 9A-9B. In preferred embodiments, cleavage of selectively cleavable linkage provides no additional chemical groups (“scars”) to the incorporated nucleic acid moiety (traceless linker cleavage).

In some embodiments, a traceless linker cleavage can be achieved as shown below, using the photolysis of 2-nitrobenzyl groups in 10 s using a 365 nm light 31 without causing DNA damage (described in detail in Litosh, V. A., et al., Improved nucleotide selectivity and termination of 3′-OH unblocked reversible terminators by molecular tuning of 2-nitrobenzyl alkylated HOMedU triphosphates. Nucleic Acids Research, 2011. 39(6): p. e39-e39; Stupi, B. P., et al., Stereochemistry ofBenzylic Carbon Substitution Coupled with Ring Modification of 2-Nitrobenzyl Groups as Key Determinants for Fast-Cleaving Reversible Terminators. Angewandte Chemie International Edition, 2012. 51(7): p. 1724-1727).

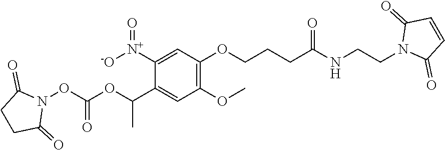

In some embodiments, a photocleavable linker containing a Maleimide and an NHS ester groups can be used to link the binder-writer conjugate and amine-modified nucleic acid moieties (e.g., nucleotide moieties). The maleimide group can react with thiol groups at slightly acidic conditions, pH between 6.5 to 7.0. The NHS ester, on the other hand, can be used in slightly basic conditions, pH between 8.3 to 8.5, to label the primary amines (—NH2) of proteins or amine-modified nucleic acid moieties (e.g., nucleotide moieties).

In some embodiments, the cleavage does not need to be traceless as it has been shown that the scared DNA (recording tag) can be efficiently PCR-amplified and sequenced (Palluk, S., et al., De novo DNA synthesis using polymerase-nucleotide conjugates. Nature Biotechnology, 2018. 36(7): p. 645-650). The scars can be avoided or minimized by changing the conjugation chemistry.

In some embodiments, during the analyzing step, an artificial intelligence (AI) model is applied to calculate probabilities of occurrence of specific types of amino acid residues in corresponding places in amino acid sequence of the peptide based on a nucleotide sequence of the further extended nucleic acid recording tag.

In some embodiments, the analyzing step comprises a nucleic acid sequencing method.

In some embodiments, the writer enzyme catalyzes covalent addition of a nucleic acid moiety to the 3′ hydroxyl of the nucleic acid recording tag.

In some embodiments, the covalent addition of nucleic acid moiety to the terminus of the nucleic acid recording tag occurs for a controlled amount of time. In some preferred embodiments, the controlled amount of time is determined by an apyrase-mediated nucleic acid moiety degradation.

Apyrases are a general class of nucleoside-triphosphate diphosphatases, so any enzyme with this activity can be used as in the disclosed methods to control concentration of a nucleic acid moiety. Some apyrases work with trinucleotide, dinucleotides and/or mononucleotides as nucleic acid moieties (e.g., nucleotide moieties). Typically, apyrase working conditions are compatible with conditions for template-independent polymerases (such as TdT) or ligases; specific parameters, such as ratio of apyrase to TdT, need to be optimized to provide desired incorporation rate.

In some embodiments, the binding agent binds to a chemically modified N-terminal amino acid residue or a chemically modified C-terminal amino acid residue. To increase the affinity of a binding agent to small N-terminal amino acids (NTAAs) of peptides, the NTAA may be modified with additional moiety or label, which may be achieved by modifying an N-terminal amino acid (NTAA) residue of the peptide with a modifying reagent. Modification of NTAA residues greatly increase chances of selection of high-affinity NTAA binding agents and simultaneously achieve good binding selectivity for NTAA of a particular type. Such affinity enhancements may be achieved with different NTAA modifiers; exemplary modifying reagents are disclosed below.

In some embodiments, the present binders can have a binding kinetics and/or affinity towards a modified target peptide comprising a specific P1 residue that is at least 2-fold or higher as compared to the binder's binding kinetics and/or affinity towards an otherwise identical modified target peptide but comprising a different P1 residue.

In some embodiments of the disclosed methods, the first binding agent and/or the second binding agent are each independently capable of specific binding to a particular type of the modified NTAA residue of the peptide. In other embodiments of the disclosed methods, the first binding agent and/or the second binding agent each independently bind with a similar affinity to at least two different modified NTAA residues of the peptide.

In some embodiments, the disclosed methods further comprise, before the analyzing step, cleaving the modified NTAA residue of the peptide, thereby generating a newly exposed NTAA residue of the peptide.

In some embodiments that involve a multi-step encoding, there is a need to distinguish on the extended recording tag a border where one encoding cycle ends and another encoding cycle starts. One possible solution for this problem is to ligate a cycle-specific oligonucleotide barcode to the terminus of the extended recording tag after each encoding cycle using a RNA ligase or a DNA ligase. Different ligases are commercially available and can be chosen based on type of the recording tag (e.g., single stranded DNA, double stranded DNA, and so on). For example, New England Biolabs offers a selection of ligases from substrate-based ligase selection chart.

In the embodiments of the disclosed methods that comprise the cleaving step, the process of identifying peptide components comprises at least two encoding cycles separated by the cleaving step. In one embodiment, binding agents used in the applied compositions recognize the modified NTAA residues of the peptide immobilized on a solid support. One encoding cycle comprise encoding information regarding a single modified NTAA residue; the cycle comprises the steps of a) modifying an N-terminal amino acid (NTAA) residue of the peptide with a modifying reagent, thereby generating a modified NTAA residue of the peptide; b) contacting the modified NTAA residue with a modified NTAA-specific binding agent (within the first composition or a mixture of compositions); c) following the binding of the binding agent to the modified NTAA residue, allowing the writer enzyme attached to the binding agent to extend nucleic acid recording tag associated with the peptide, thereby encoding information regarding identity of the binding agent into nucleic acid sequence of the extended recording tag; d) optionally, repeating steps b) and c) to perform binding of the modified NTAA residue with another binding agent (within the second composition or a mixture of compositions) having some specificity towards the modified NTAA residue; and e) cleaving the modified NTAA residue of the peptide. After that, the second encoding cycle begins, comprising similar steps, and encoding information regarding identity of the binding agent specific for the newly exposed modified NTAA residue of the peptide into nucleic acid sequence of the extended recording tag. Such encoding cycles may be repeated until all amino acid residues of the immobilized peptide are encoded and cleaved off. Then, the extended nucleic acid recording tag containing information about binding history for terminal amino acids of the peptide is subjected to analysis, such as to identify (at least partially) its sequence. Using sequence of the extended nucleic acid recording tag, one can obtain information regarding binding kinetics or selectivity of the binding agents that sequentially interacted with the modified NTAA residues of the immobilized peptide. Since only certain nucleotides are added to the terminus of the recording tag during binding agent-modified NTAA interaction in a single encoding cycle, the sequence of the extended recording tag can be used to decode identity of the amino acids of the immobilized peptide. In this embodiment, each encoding cycle is designed to identify one modified NTAA residue using a specific binder or binders; performing several consecutive encoding cycles will provide information regarding identity of several consecutive amino acid residues of the immobilized peptide.

In preferred embodiments, binding agents within the conjugates used in the disclosed methods are not specific or selective to a particular modified NTAA residue of the immobilized peptide. Instead, they may recognize and bound to several different modified NTAA residues with some affinity. In these embodiments, it is difficult to derive identity of the NTAA residue from the identity of a single binder that interacted with the modified NTAA residue. In such embodiments, several such binders from different compositions are utilized either sequentially or as a mixture in a single encoding cycle. In some preferred embodiments, each binder is selective towards a few modified NTAA residues of immobilized peptide analytes, and all binders in combination are selective towards most, if not all, of the 20 natural NTAA residues of immobilized peptide analytes modified with a chemical agent. It is preferable to have binders that have complementary selectivity, so the most NTAA residues are covered by only several binders (e.g., 4-8 binders). One binder within this set of binders would have a low dissociation rate towards a few modified NTAA residues, and another binder within this set of binders would have low dissociation rates towards different modified NTAA residues. Using only a few such binders within conjugates in the disclosed methods would allow to effectively decode NTAA residues of the immobilized peptides.

Interaction of each binder with the modified NTAA residue results in addition of a specific nucleotide (or dinucleotide, trinucleotide, etc.) to the terminus of the (extended) recording tag by the writer enzyme conjugated with the binder. In the case where interaction between the binder and the modified NTAA residue has a low dissociation rate (Kd), a few specific nucleotides (or dinucleotides) may be added sequentially to the terminus of the (extended) recording tag during the binding cycle. Similarly, interaction of several non-selective binders with the modified NTAA residue in a single encoding cycle may result in addition of specific nucleotides or small nucleotide stretches to the terminus of the recording tag by the writer enzyme conjugated with the binder. Compositions of such nucleotide stretches are determined by relative binding kinetics (e.g., rates of association and dissociation) of the binders to the modified NTAA residue (in the case where the binders were added as a mixture), or by order in which the binders were added to the immobilized peptide (in the case where the binders were added separately (sequentially) one by one and are not competing for the modified NTAA residue). FIG. 2A and FIG. 2B show an exemplary encoding cycle which comprises sequential addition of 4 different binders in 4 different compositions comprising different types of nucleotides. Binding of binder 1 to the P1 residue of the immobilized peptide results in addition of nucleotide C to the recording tag by the writer enzyme conjugated with the binder; then, binding of binder 2 to the P1 residue results in addition of nucleotide T to the recording tag; then, binding of binder 3 to the P1 residue results in addition of nucleotide G to the recording tag, and finally, binding of binder 4 to the P1 residue results in addition of nucleotide A to the recording tag. In this exemplary method, the writer enzyme attached to each of the binders 1-4 is able to add more than a single nucleotide during binding of each binder to the P1 residue. As a result, during the single encoding cycle the recording tag was extended by the following sequence: CCCCTTTTTGGGAAA, SEQ ID NO: 26 (FIG. 2B), which can be viewed as unique nucleic acid barcode specific for the particular NTAA residue of the analyzed peptide. Knowing the order in which the binders 1-4 were added, information regarding specific binding events occurred during a particular binding cycle and binding kinetics for the interacting binders can be derived from sequence of the extended recording tag.

In another example, in a given binding cycle, the nucleotide sequence tggggg is added by the writer enzyme to the recording tag associated with the immobilized peptide. Four binders are used, wherein each binder within the conjugate with the writer enzyme has a unique specificity towards modified NTAA residues of peptides and can only induce addition of a single type of nucleotide (such as binder 1 preferably binds to amino acid residues A, B and C, and can only induce addition of nucleotide “t” to the recording tag upon binding; binder 2 preferably binds to amino acid residues D, E and F, and can only induce addition of nucleotide “g” upon binding; and so on). Sequence analysis of the recording tag associated with the immobilized peptide can reveal that, in a given binding cycle, the NTAA residue of the peptide was bound only to binder 1 and binder 2, and not to binders 3 and 4. Moreover, the rate of dissociation (Kd) with the NTAA residue was lower for binder 2 than for binder 1 (since five “g” and only a single “t” were added). Therefore, it is likely that the NTAA residue in this binding cycle was residue D, E or F. If it is known that binder 1 has small affinity towards residue E, but has no affinity towards residues D and F, then E is the most probable candidate for the NTAA residue of the peptide in a given binding cycle. In this example, binders 1-4 may be added sequentially one by one, each supplemented with a corresponding nucleotide (i.e., binder 1 is supplemented with “t” (e.g., dTTP), binder 1 is supplemented with “g”, and so on). Alternatively, binders 1-4 may be added simultaneously as a mixture. To achieve specificity of the nucleotide addition upon binding, the specific nucleic acid moiety may be covalently tethered to the writer enzyme to ensure that the specific nucleic acid moiety can be added to the terminus of the recording tag. Methods for tethering specific nucleic acid moiety to the writer enzyme are disclosed, for example, in U.S. Pat. No. 11,254,961 B2, incorporated by reference herein.

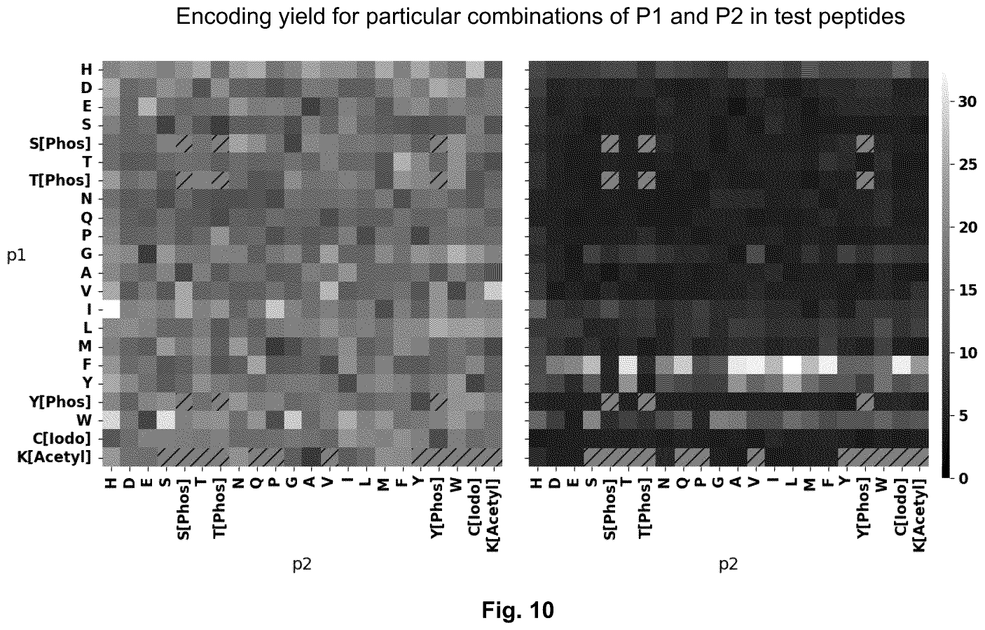

In preferred embodiments, selectivity of each binder used in conjugation with the writer enzyme during the encoding assay towards NTAA resides or modified NTAA resides of peptide analytes is determined in advance, before performing contacting steps of the disclosed methods. Each binder may be tested against a panel of peptides each having a different NTAA reside and an associated recording tag (see e.g., FIG. 10) to characterize selectivity and, optionally, binding kinetics of the binder for each of the 20 natural NTAA resides. When multiple alternative binders exist, a set comprising minimum number of binders may be selected that would cover all of the 20 natural NTAA resides.

Given the known selectivity of each of the binders in the binder set used during the disclosed methods, information regarding identity of the NTAA residue of the analyzed immobilized peptide is encoded in unique nucleic acid barcode present in the extended recording tag. This nucleic acid barcode may be used to decode the identity of the NTAA residue by using known information regarding binding kinetics and/or specificity of the binding agents bound to the peptide at a given binding cycle. In some embodiments, the nucleic acid barcode may be used as an input to a probabilistic neural network which was trained to relate the sequence of the barcode to amino acid identity. Training can be performed by testing each binder individually (conjugated to the writer enzyme) against a panel of peptides each having a different NTAA reside and an associated recording tag (see e.g., FIG. 10), collecting sequence information of the recording tags extended after the binding, and feeding the collected information to the probabilistic neural network. Alternatively, training can be performed by testing a mixture of binders (conjugated to the writer enzyme) against the panel of peptides, collecting sequence information of the recording tags extended after the binding, and feeding the collected information to the probabilistic neural network.

In some embodiments, during each encoding cycle, only single amino acid residue of the analyzed peptide gets encoded into the recording tag (each time it is an NTAA residue, which gets cleaved off at the end of each binding cycle). In other embodiments, a dipeptide gets encoded into the recording tag and dipeptides are cleaved between binding cycles (e.g., by dipeptidyl peptidases).

In some embodiments, after several cycles of encoding, each immobilized peptide is back-translated into a series of unique nucleic acid barcodes on the corresponding recording tag associated with the immobilized peptide. Each nucleic acid barcode has up to four regions of various length (x₁, x₂, x₃, x₄), wherein x₁, x₂, x₃, x₄ correspond to specific nucleotides, and each of the x₁, x₂, x₃, x₄ is added to the terminus of the recording tag only when corresponding binder interacts with the modified NTAA residue sufficiently strong enough for the writer enzyme to incorporate x₁, x₂, x₃ or x₄ into the recording tag. During the analysis step, sequence of the extended recording tag can be analyzed to extract the abovementioned nucleic acid barcodes that correspond to each encoding cycle. Then, to associate the extracted nucleic acid barcodes with corresponding amino acid residues, an artificial intelligence (AI) model can be applied to calculate probabilities of occurrence of specific types of amino acid residues in corresponding places in amino acid sequence of the analyzed peptide. In preferred embodiments, the AI model can be pre-trained using multiple known peptide sequences, which were used to generate encoding nucleic acid data on associated recording tags. Modeling encoding of multiple known peptides using known writer-binder conjugates allows for training the AI model to faithfully predict amino acid residues based on provided barcode nucleic acid sequences.

In some embodiments, the generated DNA barcodes are input to a probabilistic neural network (PNN) which will learn to relate the sequence of a DNA barcode to an amino acid identity. Probabilistic neural networks (Mohebali, B., et al., Chapter 14—Probabilistic neural networks: a brief overview of theory, implementation, and application, in Handbook of Probabilistic Models, P. Samui, et al., Editors. 2020, Butterworth-Heinemann. p. 347-367) can approach Bayes optimal classification for multiclass problems such as amino acid identification from DNA barcodes (Klocker, J., et al., Bayesian Neural Networks for Aroma Classification. Journal of Chemical Information and Computer Sciences, 2002. 42(6): p. 1443-1449). A classifier based on PNN is guaranteed to learn and converge to an optimal classifier as the size of the representative data set increases. Probabilistic neural networks have parallel structure such that data from any amino acid residue are used to learn all other amino acid residues.

In some embodiments, the disclosed methods are used for peptide sequence determination based on probabilistic neural network ensembles. The machine learning method is characterized in that the sequence determination can be realized by the following steps: i) the peptide fragments of proteins are encoded using binder-writer conjugates into stretches of DNA sequences based on the physicochemical properties of amino acid residues; ii) a group of probabilistic neural network sub-classifiers are established, peptide fragments of proteins with known sequence are used to perform amino acid classification training and obtain a group of trained amino acid classification models; iii) the obtained models are utilized to determine peptide amino acid sequences in the test data sets; iv) the classification results output by the models are counted to generate amino acid candidate sets; v) the methods showing highest accuracy are combined to determine the amino acid sequence of protein peptide fragment; and vi) the algorithmic amino acid determination result is verified through k-fold cross-validation, where k is an integer.

In some embodiments, k-fold cross-validation operates as follows. In k-fold cross-validation, the dataset is shuffled and divided into k groups randomly with no overlap and replacements. This means each group is unique and is used for model evaluation only once. The data groups are carried through the following steps to perform the k-fold cross-validation:
1) A unique group is taken as a test data set;
2) The remaining (k−1) groups are used as a training data set;
3) A model is built using the training set and is evaluated using the test set;
4) The evaluation score is retained and the model is discarded;
5) Step 1-4 are repeated until all k groups are used for model evaluation;
6) The mean of the k evaluation scores is output as the k-fold cross-validated model performance score.

In some embodiments, the nucleic acid barcodes are input to a probabilistic neural network (PNN), which will learn to relate the DNA sequence of a barcode to an amino acid identity of the analyzed peptide. In other embodiments, other statistical models (e.g., hidden Markov models) and machine learning methods (e.g., random forest models) can be used for classifying a NGS read from extended recording tag into a specific amino acid residue (or amino acid residue type, if binder is not selective to specific amino acid residues).

In some embodiments, to bioinformatically distinguish within the sequence of the extended recording tag borders of barcodes corresponding to different encoding cycles (where one encoding cycle ends and another encoding cycle starts), a special short nucleic acid sequence may be added at the end of each encoding cycle (e.g., after the modified NTAA residue is cleaved off the immobilized peptide and new NTAA residue is exposed). For example, a specific dinucleotide may be added (ligated) to the terminus of the extended recording tag at the end of each encoding cycle by using, for example, a T4 RNA ligase. Then, during sequence analysis of the extended recording tag it is easy to separate barcode sequences that correspond to different encoding cycles and encode different amino acids.

In preferred embodiments of the disclosed methods, encoding of NTAA information can be achieved by utilizing a plurality of conjugates, wherein each conjugate comprises a binding agent capable of binding to the peptide and a writer enzyme (a writer) capable of catalyzing covalent addition of a nucleic acid moiety to a terminus of the nucleic acid recording tag, wherein the binding agent is conjugated to the writer. In preferred embodiments, the writer enzyme comprises a template-independent polymerase (such as Terminal deoxynucleotidyl Transferase (TdT)), a DNA ligase, or a RNA ligase.

In some embodiments of the disclosed methods, kinetics of binder-peptide interaction is encoded into a single or double stranded-stranded DNA barcode (see e.g., FIG. 1A). Each kinetic encoder is a chimeric protein comprising a conjugate of a binder and a writer. The writer is a template-independent polymerase or ligase such as terminal deoxynucleotidyl transferase (TdT) domain or T4 RNA ligase (FIG. 1A) (Tessier, D. C., et. al., Ligation of single-stranded oligodeoxyribonucleotides by T4 RNA ligase. Analytical Biochemistry, 1986. 158(1): p. 171-178). Under kinetically controlled conditions, the writer encodes the identity and kinetic information of the binder-peptide interactions into nucleic acid barcodes comprising short tandem repeats (STRs) (see FIG. 2A-2B). The writer enzyme enables capturing kinetic information of binder-peptide interaction.

In some embodiments, the writer enzyme is TdT—a distributive polymerase (see. e.g., U.S. Pat. No. 10,760,063 B2). It, at most, adds one nucleotide in each encounter with a DNA strand (e.g., with the recording tag associated with the immobilized peptide analyte). Therefore, the kinetics of TdT-mediated elongation of a single-stranded recording tag depends on the local concentration and proximity of TdT to the terminus of the recording tag. The fusion (conjugation) of TdT to the binder enables it to record the kinetic information of the binder-peptide interaction onto the adjacent recording tag strand. The lengths of produced STRs depend on the rate of the slowest (rate-determining) step. For example, if the rate-determining step is expected to be the dissociation of binder from the peptide, the length of each STR should be inversely proportional to the kinetic constant of the binder-peptide dissociation, k_{off}. The off rate, k_{off}, and hence residence time of binder-peptide interaction is first-order and independent of binder's concentration.

In some preferred embodiments, the identity and length of oligonucleotide(s) installed by the writer onto the terminus of the recording tag contain information about the identity and kinetics of the binding, respectively. In some embodiments, each N-terminal amino acid (NTAA) residue of the immobilized peptide analyte is encoded stepwise by four different binders of orthogonal physicochemical binding propensity in each encoding cycle. Recording the kinetics of binding with multiple specific kinetic encoders will produce a unique barcode for each N-terminal amino acid. In some embodiments, the length of each encoding cycle is controlled by apyrase-mediated dNTP degradation. Alternatively, in other embodiments, encoding of NTAA residue information with multiple kinetic encoders can be achieved in a single step using a set of binder-writer conjugates pre-loaded with (tethered to) nucleic acid moieties (e.g., nucleotide moieties) (as disclosed in U.S. Pat. No. 11,254,961 B2, incorporated herein). Ideally, each NTAA residue of the immobilized peptide analyte is encoded by four or more distinct kinetic encoders (binder-writer conjugates) that have orthogonal residence time on different physicochemical classes of NTAA residues.

In some embodiments, T4 RNA ligase is used as a writer in binder-writer conjugate(s) used for encoding; in such embodiments, nucleic acid moieties (e.g., nucleotide moieties) comprising dinucleotide 5′-triphosphates can be configured to be incorporated to the terminus of the recording tag associated with the immobilized peptide analyte (Torchia, et al., Archaeal RNA ligase is a homodimeric protein that catalyzes intramolecular ligation of single-stranded RNA and DNA. Nucleic Acids Research, 2008. 36(19): p. 6218-6227; England, et al., Dinucleoside pyrophosphate are substrates for T4-induced RNA ligase. Proceedings of the National Academy of Sciences, 1977. 74(11): p. 4839-4842; Zhelkovsky, A. M. and L. A. McReynolds, Structure-function analysis of Methanobacterium thermoautotrophicum RNA ligase—engineering a thermostable ATP independent enzyme. BMC Molecular Biology, 2012. 13(1): p. 24. Different combination of dinucleotide 5′-triphosphates as nucleic acid moieties (e.g., nucleotide moieties) allows for using up to 16 unique binder-writer conjugates. By increasing the number of nucleotide bases in the nucleic acid moieties (e.g., nucleotide moieties) configured to be incorporated to the terminus of the recording tag, an increased number of binder-writer conjugates can be used as a set in the encoding assay.

In some embodiments, providing the peptide and an associated recording tag joined to a solid support comprises the following steps: attaching the peptide to the recording tag to generate a nucleic acid-peptide conjugate; bringing the nucleic acid-peptide conjugate into proximity with a solid support by hybridizing the recording tag in the nucleic acid-peptide conjugate to a capture nucleic acid attached to the solid support; and covalently coupling the nucleic acid-peptide conjugate to the solid support. Preferred immobilization methods of the peptide and an associated recording tag on the solid support are disclosed in US 2022/0049246 A1, incorporated herein.

Recording tags can be attached to the peptide pre- or post-immobilization to the solid support. For example, peptides can be first labeled with recording tags and then immobilized to a solid surface via a recording tag comprising two functional moieties for coupling. One functional moiety of the recording tag couples to the peptide, and the other functional moiety immobilizes the recording tag-labeled peptide to a solid support. Alternatively, peptides are immobilized to a solid support prior to labeling with recording tags. For example, peptides can first be derivatized with reactive groups such as click chemistry moieties. The activated peptides molecules can then be attached to a suitable solid support and then labeled with recording tags using the complementary click chemistry moiety. As an example, peptides derivatized with alkyne and mTet moieties may be immobilized to beads derivatized with azide and TCO and attached to recording tags labeled with azide and TCO. It is understood that the methods provided herein for attaching peptides to the solid support may also be used to attach recording tags to the solid support or attach recording tags to peptides.

A peptide and an associated recording tag can be joined to the solid support, directly or indirectly (e.g., via a linker), by any means known in the art, including covalent and non-covalent interactions, or any combination thereof. For example, the recording tag may be joined to the solid support by a ligation reaction. Alternatively, the solid support can include an agent or coating to facilitate joining, either direct or indirectly, of the recording tag, to the solid support. Strategies for immobilizing nucleic acid molecules to solid supports (e.g., beads) have been described in U.S. Pat. No. 5,900,481; Steinberg et al. (2004, Biopolymers 73:597-605), incorporated herein by reference in its entirety.

The recording tags may be associated or attached, directly or indirectly to the peptides using any suitable means. In some embodiments, a peptide may be associated with one or more recording tags. In some aspects, the recording tags may be associated or attached, directly or indirectly to the peptides prior to contacting with a binding agent.

In some embodiments, at least one recording tag is associated or co-localized directly or indirectly with the peptide. Providing a peptide and an associated recording tag may include treating the recording tag and any associated nucleic acids to join, cleave, or otherwise prepare the recording tag for the assay. In some embodiments, providing a peptide and an associated recording tag includes using ligation and/or extension to provide the barcode and/or the UMI to the recording tag.

In some embodiments, the peptide is attached to a bait nucleic acid to form a nucleic acid-peptide chimera. The immobilization methods may comprise bringing the nucleic acid-peptide chimera into proximity with a support by hybridizing the bait nucleic acid to a capture nucleic acid attached to the support, and covalently coupling the nucleic acid-peptide chimera to the support. In some cases, the nucleic acid-peptide chimera is coupled indirectly to the solid support, such as via a linker. In some embodiments, a plurality of the nucleic acid-peptide chimeras is coupled on the support and any adjacently coupled nucleic acid-peptide chimeras are spaced apart from each other at an average distance of about 50 nm or greater.

In some embodiments, the peptide is attached to the 3′ end of the recording tag. In other embodiments, the peptide is attached to the 5′ end of the recording tag. In yet other embodiments, the peptide is attached to an internal position of the recording tag.

In some embodiments, a barcode is attached to the nucleic acid-peptide conjugate, wherein the barcode comprises a compartment barcode, a partition barcode, a sample barcode, a fraction barcode, or any combination thereof.

In some embodiments, the recording tag is covalently attached to the peptide to generate the nucleic acid-peptide conjugate. In some embodiments, the recording tag and/or capture nucleic acid further comprises a universal priming site, wherein the universal priming site comprises a priming site for amplification, sequencing, or both.

In some embodiments, the capture nucleic acid is derivatized or comprises a moiety (e.g., a reactive coupling moiety) to allow binding to a solid support. In some embodiments, the capture nucleic acid comprises a moiety (e.g., a reactive coupling moiety) to allow binding to the recording tag. In some other embodiments, the recording tag is derivatized or comprises a moiety (e.g., a reactive coupling moiety) to allow binding to a solid support. Methods of derivatizing a nucleic acid for binding to a solid support and reagents for accomplishing the same are known in the art. For this purpose, any reaction which is preferably rapid and substantially irreversible can be used to attach nucleic acids to the solid support. The capture nucleic acid may be bound to a solid support through covalent or non-covalent bonds. In a preferred embodiment, the capture nucleic acid is covalently bound to biotin to form a biotinylated conjugate. The biotinylated conjugate is then bound to a solid surface, for example, by binding to a solid, insoluble support derivatized with avidin or streptavidin. The capture nucleic acid can be derivatized for binding to a solid support by incorporating modified nucleic acids in the loop region. In other embodiments, the capture moiety is derivatized in a region other than the loop region.

Exemplary bioorthogonal reactions that can be used for binding to a solid support or for generating nucleic acid-peptide conjugates include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing peptides to a solid support or for generating nucleic acid-peptide conjugates since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction.

In some embodiments, a plurality of capture nucleic acids are coupled to the solid support. In some cases, the sequence region that is complementary to the recording tag on the capture nucleic acids is the same among the plurality of capture nucleic acids. In some cases, the recording tag attached to various peptides comprises the same complementary sequence to the capture nucleic acid.

In some embodiments, the surface of the solid support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moieties (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of peptides (e.g., proteins, peptide, or peptides) can be titrated on the surface or within the volume of a solid substrate by spiking a competitor or “dummy” reactive molecule when immobilizing the proteins, peptides or peptides to the solid substrate. In some embodiments, PEGs of various molecular weights can also be used for passivation from molecular weights of about 300 Da to 50 kDa or more.

In certain embodiments where multiple recording tag-peptide conjugates are immobilized on the same solid support, the recording tag-peptide conjugates can be spaced appropriately to accommodate methods of identification disclosed herein. For example, it may be advantageous to space the recording tag-peptide conjugates apart from each other to prevent the writer enzyme to catalyze covalent addition of a nucleic acid moiety to a non-cognate recording tag (e.g., the recording tag associated with an adjacent peptide analyte). In some embodiments, recording tag-peptide conjugates immobilized on the same solid support are spaced apart at an average distance of about 50 nm or greater.

In some embodiments, a plurality of capture nucleic acids are coupled to the solid support, and the recording tag-peptide conjugates immobilized on the same solid support by nucleic acid hybridization with the capture nucleic acids. In some of these embodiments, capture nucleic acids are spaced apart at an average distance of about 50 nm or greater.

To control peptide analyte spacing on the solid support, the density of functional coupling groups (e.g., TCO or capture DNA molecules) may be titrated on the substrate surface. In some embodiments, multiple peptides are spaced apart on the surface or within the volume (e.g., porous supports) of a solid support at a distance of about 50 nm to about 500 nm, or about 50 nm to about 400 nm, or about 50 nm to about 300 nm, or about 50 nm to about 200 nm, or about 50 nm to about 100 nm. In some embodiments, multiple peptides are spaced apart on the surface or within the volume of a solid support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple peptides are spaced apart on the surface or within the volume of a solid support with an average distance of at least 50 nm. In some embodiments, peptides are spaced apart on the surface or within the volume of a solid support such that, empirically, the relative frequency of inter- to intra-molecular events is <1:10; <1:100; <1:1,000; or <1:10,000. A suitable spacing frequency can be determined empirically using a functional assay (as described, for example, in the published patent application US 2019/0145982 A1), and can be accomplished by dilution and/or by spiking a “dummy” spacer molecule that competes for attachments sites on the substrate surface. In some embodiments, when a plurality of the nucleic acid-peptide conjugates is coupled, any nucleic acid-peptide conjugates adjacently coupled on the solid support are spaced apart from each other at an average distance of about 50 nm or greater.

In some embodiments, the spacing of the peptide on the solid support is achieved by controlling the concentration and/or number of capture nucleic acids on the solid support. In some embodiments, any adjacently coupled capture nucleic acids are spaced apart from each other on the surface or within the volume (e.g., porous supports) of a solid support at a distance of about 50 nm, about 100 nm, or about 200 nm. In some embodiments, any adjacently coupled capture nucleic acids are spaced apart from each other on the surface of a solid support with an average distance of at least 50 nm. In some embodiments, any adjacently coupled capture nucleic acids are spaced apart from each other on the surface or within the volume of a solid support such that, empirically, the relative frequency of inter- to intra-molecular events (e.g., transfer of information) is <1:10; <1:100; <1:1,000; or <1:10,000.

A suitable spacing frequency can be determined empirically using a functional assay and can be accomplished by dilution and/or by spiking a “dummy” spacer molecule that competes for attachments sites on the substrate surface. For example, PEG-5000 (MW˜5000) is used to block the interstitial space between peptides on the substrate surface (e.g., bead surface). In addition, the peptide is coupled to a functional moiety that is also attached to a PEG-5000 molecule. In some embodiments, the functional moiety is an aldehyde, an azide/alkyne, or a malemide/thiol, or an epoxide/nucleophile, or an inverse electron demand Diels-Alder (iEDDA) group, or a moiety for a Staudinger reaction. In some embodiments, the functional moiety is an aldehyde group. In a preferred embodiment, this is accomplished by coupling a mixture of NHS-PEG-5000-TCO+NHS-PEG-5000-Methyl to amine-derivatized beads. The stoichiometric ratio between the two PEGs (TCO vs. methyl) is titrated to generate an appropriate density of functional coupling moieties (TCO groups) on the substrate surface; the methyl-PEG is inert to coupling. The effective spacing between TCO groups can be calculated by measuring the density of TCO groups on the surface. In certain embodiments, the mean spacing between coupling moieties (e.g., TCO) on the solid surface is at least 50 nm, at least 100 nm, at least 250 nm, or at least 500 nm. After PEG5000-TCO/methyl derivatization of the beads, the excess NH₂ groups on the surface are quenched with a reactive anhydride (e.g. acetic or succinic anhydride).

In some embodiments, the spacing is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH₂ and NH₂-PEGₙ-mTet is added to the activated beads (wherein n is any number, e.g., any number from n=1 to n=100 or more). In one example, the ratio between the mPEG₃-NH₂ (not available for coupling) and NH₂-PEG₄-mTet (available for coupling) is titrated to generate an appropriate density of functional moieties available to attach the peptide on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some specific embodiments, the ratio of NH₂-PEGₙ-mTet to mPEGₙ-NH₂ is about or greater than 1:1000, about or greater than 1:10,000, about or greater than 1:100,000, or about or greater than 1:1,000,000. In some further embodiments, the capture nucleic acid attaches to the NH₂-PEGₙ-mTet.

In certain embodiments, a recording tag comprises an optional unique molecular identifier (UMI), which provides a unique identifier tag for each peptide to which the UMI is associated with. A UMI can be about 3 to about 20 bases, or about 3 to about 8 bases in length. A UMI can be used to de-convolute sequencing data from a plurality of extended recording tags to identify sequence reads from individual peptides. In some embodiments, within a library of peptides, each peptide is associated with a single recording tag, with each recording tag comprising a unique UMI. In other embodiments, multiple copies of a recording tag are associated with a single peptide, with each copy of the recording tag comprising the same UMI.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′-SEQ ID NO: 1) or an Illumina P7 primer (5′-CAAGCAGAAGACGGCATACGAGAT-3′-SEQ ID NO:2).

In some examples, the labeling of the peptide with a recording tag is performed using standard amine coupling chemistries. In a particular embodiment, the recording tag can comprise a reactive moiety (e.g., for conjugation to a solid surface, a multifunctional linker, or a peptide), a linker, a universal priming sequence, a barcode, an optional UMI, and a spacer (Sp) sequence for facilitating information transfer to/from a coding tag. In another embodiment, the protein is labeled with a universal DNA tag prior to proteinase digestion into peptides. The universal DNA tags on the labeled peptides from the digest can then be converted into an informative and effective recording tag. A universal DNA tag comprises a short sequence of nucleotides that are used to label a peptide macromolecule and can be used as point of attachment. For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged peptide.

The recording tags may comprise a reactive moiety for a cognate reactive moiety present on the target peptide (e.g., click chemistry labeling, photoaffinity labeling). For example, recording tags may comprise an azide moiety for interacting with alkyne-derivatized proteins, or recording tags may comprise a benzophenone for interacting with native peptide. Upon binding of the target peptide by the coding tags, the recording tag and target peptide are coupled via their corresponding reactive moieties. In some embodiments, other types of linkages besides nucleic acid hybridization can be used to link the recording tag to a peptide. A suitable linker can be attached to various positions of the recording tag, such as the 3′ end, at an internal position, or within the linker attached to the 5′ end of the recording tag.

Extended nucleic acids recording tags can be processed and analyzed using a variety of nucleic acid sequencing methods. Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing.

Suitable sequencing methods for use in the invention include, but are not limited to, sequencing by hybridization, sequencing by synthesis technology (e.g., HiSeq™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeq™, Illumina), massively parallel semiconductor sequencing (e.g., Ion Torrent), pyrosequencing technology (e.g., GS FLX and GS Junior Systems, Roche/454), nanopore sequence (e.g., Oxford Nanopore Technologies).

The methods disclosed herein can be used for analysis, including detection, quantitation and/or sequencing, of a plurality of peptides simultaneously (multiplexing). Multiplexing as used herein refers to analysis of a plurality of peptides in the same assay. The plurality of peptides can be derived from the same sample or different samples. The plurality of peptides can be derived from the same subject or different subjects. The plurality of peptides that are analyzed can be different peptides, or the same peptide derived from different samples. A plurality of peptides includes 2 or more peptides, 10 or more peptides, 50 or more peptides, 100 or more peptides, 1,000 or more peptides, 5,000 or more peptides, 10,000 or more peptides, 100,000 or more peptides, or 1,000,000 or more peptides. In some embodiments, the described analysis and peptide identification are performed in parallel for multiple analyzed peptides. Following sequencing of the extended recording tags, the resulting sequences can be collapsed by their UMIs and then associated to their corresponding peptides and aligned to the totality of the peptides in the cell.

### EXEMPLARY EMBODIMENTS

Among the provided embodiments are the following enumerated embodiments:

1. A method for identifying a component of a peptide, comprising the steps of:
a. providing the peptide and an associated nucleic acid recording tag joined to a solid support;
b. contacting the peptide with a first composition comprising a first conjugate and a first nucleic acid moiety, wherein the first conjugate comprises a first binding agent capable of binding to the peptide, wherein the first binding agent is conjugated to a writer enzyme capable of catalyzing covalent addition of the first nucleic acid moiety to a terminus of the nucleic acid recording tag;
c. following the binding of the first conjugate to the peptide, allowing the writer enzyme of the first conjugate to catalyze covalent addition of the first nucleic acid moiety to the terminus of the nucleic acid recording tag to generate an extended nucleic acid recording tag joined to the solid support;
d. contacting the peptide with a second composition comprising a second conjugate and a second nucleic acid moiety, wherein the second conjugate comprises a second binding agent capable of binding to the peptide, wherein the second binding agent is conjugated to a writer enzyme capable of catalyzing covalent addition of the second nucleic acid moiety onto a terminus of the extended nucleic acid recording tag;
e. following the binding of the second conjugate to the peptide, allowing the writer enzyme of the second conjugate to catalyze covalent addition of the second nucleic acid moiety to the terminus of the extended nucleic acid recording tag to generate a further extended nucleic acid recording tag joined to the solid support;
f. optionally, repeating steps (d)-(e) one or more times by replacing the second composition with a third or higher order composition comprising a third or higher order conjugate and a third or higher order nucleic acid moiety, wherein the third or higher order conjugate comprises a third or higher order binding agent capable of binding to the peptide conjugated to a writer enzyme capable of catalyzing covalent addition of the third or higher order nucleic acid moiety onto a terminus of the extended or further extended nucleic acid recording tag; and by allowing the writer enzyme of the third or higher order conjugate to catalyze covalent addition of the third or higher order nucleic acid moiety to the terminus of nucleic acid recording tag extended after step (e) or after previous addition(s) to generate a further extended nucleic acid recording tag joined to the solid support; and
g. analyzing the further extended nucleic acid recording tag and obtaining information regarding binding kinetics and/or selectivity of the first binding agent binding to the peptide and information regarding binding kinetics and/or selectivity of the second binding agent binding to the peptide, thereby identifying a component of the peptide.

2. The method of embodiment 1, wherein the contacting in steps (b) and (d) are performed in a sequential order.

3. The method of embodiment 1, wherein the contacting in steps (b) and (d) are performed at the same time.

4. The method of any one of embodiments 1-3, wherein a plurality of peptides is provided at step (a), each peptide from the plurality of peptides is independently associated with a nucleic acid recording tag (which can be the same or different for any two or more molecules of any one or more peptides of the plurality of peptides) joined to a solid support, and wherein the plurality of peptides is contacted with the first composition at step (b) and with the second composition at step (d).

5. The method of embodiment 4, wherein the plurality of peptides comprises at least 100 peptides.

6. The method of embodiment 5, wherein components of at least 100 peptides are identified during the analyzing step.

7. The method of any one of embodiments 1-6, further comprising, before step (b), modifying an N-terminal amino acid (NTAA) residue of the peptide with a modifying reagent, thereby generating a modified NTAA residue of the peptide.

8. The method of embodiment 7, wherein the first binding agent and/or the second binding agent are each independently capable of specific binding to a particular type of the modified NTAA residue of the peptide.

9. The method of embodiment 7, wherein the first binding agent and/or the second binding agent each independently bind with substantially the same or a similar affinity to at least two different modified NTAA residues of the peptide.

10. The method of embodiment 7, further comprising, before the analyzing step, cleaving the modified the NTAA residue of the peptide, thereby generating a newly exposed NTAA residue of the peptide.

11. The method of any one of embodiments 1-10, wherein the writer enzyme is a template-independent polymerase, a DNA ligase, or a RNA ligase.

12. The method of embodiment 11, wherein the template-independent polymerase is a Terminal deoxynucleotidyl Transferase (TdT).

13. The method of embodiment 11, wherein the first composition and/or the second composition comprise the first nucleic acid moiety and/or the second nucleic acid moiety covalently tethered to the template-independent polymerase, the DNA ligase, or the RNA ligase via a second linker comprising a selectively cleavable linkage.

14. The method of any one of embodiments 1-13, wherein the first binding agent and/or the second binding agent is conjugated to the writer enzyme via a first linker.

15. The method of embodiment 14, wherein the first linker is selected from the group consisting of: a peptide, poly alkyl chain (CH₂)ₙ polymer, a poly(ethylene glycol) PEG polymer, a poly(ethylene oxide) PEO polymer, and a combination thereof.

16. The method of any one of embodiments 1-15, wherein during the analyzing step, an artificial intelligence (AI) model, e.g., an AI model employing probabilistic neural networks (PNN), is applied to calculate probabilities of occurrence of specific types of amino acid residues in corresponding places in amino acid sequence of the peptide based on a nucleotide sequence of the further extended nucleic acid recording tag.

17. The method of any one of embodiments 1-16, wherein the writer enzyme catalyzes covalent addition of the first nucleic acid moiety and/or the second nucleic acid moiety to the 3′ hydroxyl of the nucleic acid recording tag.

18. The method of any one of embodiments 1-17, wherein the covalent addition of the first nucleic acid moiety and/or the second nucleic acid moiety to the terminus of the nucleic acid recording tag occurs for a controlled amount of time.

19. The method of embodiment 18, wherein the controlled amount of time is achieved by using an apyrase-mediated nucleoside degradation.

20. The method of any one of embodiments 1-19, wherein the analyzing step comprises a nucleic acid sequencing method.

21. A method for identifying a component of a peptide, comprising the steps of:
a. providing the peptide and an associated nucleic acid recording tag joined to a solid support;
b. contacting the peptide with a mixture comprising a first composition, a second composition, and, optionally, a third or higher order composition, wherein (i) the first composition comprises a first conjugate and a first nucleic acid moiety; the first conjugate comprises a first binding agent capable of binding to the peptide; the first binding agent is conjugated to a writer enzyme capable of catalyzing covalent addition of the first nucleic acid moiety to a terminus of the nucleic acid recording tag; and the first nucleic acid moiety is covalently tethered to the writer enzyme via a linker comprising a selectively cleavable linkage; (ii) the second composition comprises a second conjugate and a second nucleic acid moiety; the second conjugate comprises a second binding agent capable of binding to the peptide; the second binding agent is conjugated to a writer enzyme capable of catalyzing covalent addition of the second nucleic acid moiety to a terminus of the nucleic acid recording tag; and the second nucleic acid moiety is covalently tethered to the writer enzyme via a linker comprising a selectively cleavable linkage; (iii) the third or higher order composition comprises a third or higher order conjugate and a third or higher order nucleic acid moiety; the third or higher order conjugate comprises a third or higher order binding agent capable of binding to the peptide; the third or higher order binding agent is conjugated to a writer enzyme capable of catalyzing covalent addition of the third or higher order nucleic acid moiety to a terminus of the nucleic acid recording tag; and the third or higher order nucleic acid moiety is covalently tethered to the writer enzyme via a linker comprising a selectively cleavable linkage;
c. following binding of the first conjugate, the second conjugate or the third or higher order conjugate to the peptide, allowing the writer enzyme of the conjugate bound to the peptide to catalyze covalent addition of the nucleic acid moiety covalently tethered to the writer enzyme to the terminus of the nucleic acid recording tag to generate an extended nucleic acid recording tag joined to the solid support;
d. cleaving the selectively cleavable linkage and releasing the writer enzyme from the nucleic acid moiety on the terminus of the extended nucleic acid recording tag;
e. optionally, repeating steps (b)-(d) one or more times to generate a further extended nucleic acid recording tag joined to the solid support; and
f. analyzing the extended nucleic acid recording tag or the further extended nucleic acid recording tag and obtaining information regarding binding kinetics and/or selectivity of the binding agent(s) bound to the peptide, thereby identifying a component of the peptide.

22. The method of embodiment 21, wherein the analyzing step comprises a nucleic acid sequencing method.

23. The method of embodiment 21 or embodiment 22, wherein during the analyzing step, an artificial intelligence (AI) model, e.g., an AI model employing probabilistic neural networks (PNN), is applied to calculate probabilities of occurrence of specific types of amino acid residues in corresponding places in amino acid sequence of the peptide based on a nucleotide sequences of the extended and/or the further extended nucleic acid recording tag(s).

24. The method of any one of embodiments 21-23, wherein the writer enzyme catalyzes covalent addition of a nucleic acid moiety to the 3′ hydroxyl of the nucleic acid recording tag.

25. The method of any one of embodiments 21-24, wherein the covalent addition of a nucleic acid moiety to the terminus of the nucleic acid recording tag occurs for a controlled amount of time.

26. The method of embodiment 25, wherein the controlled amount of time is achieved by using an apyrase-mediated nucleoside degradation.

27. The method of any one of embodiments 21-26, further comprising, before the contacting step (b), modifying an N-terminal amino acid (NTAA) residue of the peptide with a modifying reagent, thereby generating a modified NTAA residue of the peptide.

28. The method of embodiment 27, wherein the first binding agent and/or the second binding agent are each independently capable of specific binding to a particular type of the modified NTAA residue of the peptide.

29. The method of embodiment 27, wherein the first binding agent and/or the second binding agent each independently bind with substantially the same or a similar affinity to at least two different modified NTAA residues of the peptide.

30. The method of any one of embodiments 27-29, further comprising, before the analyzing step, cleaving the modified NTAA residue of the peptide, thereby generating a newly exposed NTAA residue of the peptide.

31. The method of any one of embodiments 21-30, wherein the writer enzyme is a template-independent polymerase, a DNA ligase, or a RNA ligase.

32. The method of embodiment 31, wherein the template-independent polymerase is a Terminal deoxynucleotidyl Transferase (TdT).

33. A conjugate, which comprises a binding agent conjugated via a first linker to a writer enzyme, wherein said conjugate is configured to bind to a peptide comprising an associated nucleic acid recording tag joined to a solid support, said binding agent is configured to bind to said peptide, and said writer enzyme is configured to catalyze covalent addition of a nucleic acid moiety onto a terminus of said nucleic acid recording tag.

34. The conjugate of embodiment 33, which further comprises a nucleic acid moiety that is covalently tethered to the writer enzyme via a second linker comprising a selectively cleavable linkage.

35. The conjugate of embodiment 34, wherein the second linker comprises alkyl, PEG, or PEO moiety with 2-18 chain lengths.

36. A composition comprising a plurality of the conjugates of embodiments 33, 34 and/or 35.

37. A kit for identifying a component of a peptide, which kit comprises a conjugate of any of embodiments 33-35, or a composition of embodiment 36, and an instruction for using the conjugate or the composition for identifying the component of the peptide.

38. The method of any one of embodiments 1-32, wherein the binding agent is a polypeptide, e.g., an antibody, antibody fragment or an engineered binder, and both the polypeptide binding agent and the writer enzyme are parts of a conjugate.

39. The conjugate of any one of embodiments 33-35, wherein the binding agent is a polypeptide, e.g., an antibody, antibody fragment or an engineered binder, and both the polypeptide binding agent and the writer enzyme are parts of the conjugate.

40. The composition of embodiment 36, wherein the binding agent is a polypeptide, e.g., an antibody, antibody fragment or an engineered binder, and both the polypeptide binding agent and the writer enzyme are parts of a conjugate.

41. The kit of embodiment 37, wherein the binding agent is a polypeptide, e.g., an antibody, antibody fragment or an engineered binder, and both the polypeptide binding agent and the writer enzyme are parts of a conjugate.

42. A method for analyzing a peptide, wherein the peptide and an associated nucleic acid recording tag are joined to a support, the method comprising:
a) contacting the peptide with a first composition comprising a first conjugate and a first nucleic acid moiety, wherein the first conjugate comprises a first binding agent that binds to the peptide, wherein the first binding agent is conjugated to a first writer enzyme that catalyzes covalent addition of the first nucleic acid moiety to a terminus of the nucleic acid recording tag to generate an extended nucleic acid recording tag joined to the support;
b) contacting the peptide with a second composition comprising a second conjugate and a second nucleic acid moiety, wherein the second conjugate comprises a second binding agent that binds to the peptide, wherein the second binding agent is conjugated to a second writer enzyme capable of catalyzing covalent addition of the second nucleic acid moiety to a terminus of the extended nucleic acid recording tag to generate a further extended nucleic acid recording tag joined to the support; and
c) analyzing the further extended nucleic acid recording tag to obtain information regarding binding kinetics and/or selectivity of the first binding agent binding to the peptide and information regarding binding kinetics and/or selectivity of the second binding agent binding to the peptide, thereby analyzing the peptide.

43. The method of embodiment 42, further comprising: contacting the peptide with a third composition comprising a third conjugate and a third nucleic acid moiety, wherein the third conjugate comprises a third binding agent that binds to the peptide, wherein the third binding agent is conjugated to a third writer enzyme that catalyzes covalent addition of the third nucleic acid moiety to a terminus of the further extended nucleic acid recording tag to generate an even further extended nucleic acid recording tag joined to the support, and analyzing the even further extended nucleic acid recording tag to obtain information regarding binding kinetics and/or selectivity of the third binding agent binding to the peptide.

44. The method of embodiment 42 or 43, wherein the contacting in (a) and the contacting in (b) are performed in a sequential order.

45. The method of embodiment 42 or 43, wherein the contacting in (a) and the contacting in (b) are performed at the same time.

46. The method of any one of embodiments 42-45, wherein a plurality of peptides are contacted with the first composition in (a) and with the second composition in (b), and wherein each peptide from the plurality of peptides is independently associated with a nucleic acid recording tag (which can be the same or different for any two or more molecules of any one or more peptides of the plurality of peptides), and each peptide and the associated nucleic acid recording tag is joined to the support. In some of these embodiments, peptides of plurality of peptides are spaced apart from each other at an average distance of about 50 nm or greater.

47. The method of embodiment 46, wherein two or more peptides from the plurality of peptides comprise different amino acid sequences.

48. The method of embodiment 46 or 47, wherein two or more peptides from the plurality of peptides comprise a common amino acid sequence.

49. The method of any one of embodiments 45-48, wherein the plurality of peptides comprises at least 100, 1000, 10000, 100000 or more peptides.

50. The method of any one of embodiments 45-49, wherein a sequence of each peptide from the plurality of peptides is identified.

51. The method of embodiment 50, wherein the identified sequence comprises a terminal amino acid residue.

52. The method of any one of embodiments 42-51, further comprising, before (a), modifying an N-terminal amino acid (NTAA) residue of the peptide with a modifying reagent, thereby generating a modified NTAA residue of the peptide.

53. The method of any one of embodiments 42-52, wherein the first binding agent and/or the second binding agent is each independently capable of specific binding to one or more unmodified or modified terminal amino acid residues, optionally wherein the one or more unmodified or modified terminal amino acid residues are unmodified or modified NTAA residue(s).

54. The method of embodiment 42, wherein the first binding agent binds to a terminal amino acid (TAA) or a modified TAA of the peptide, and the second binding agent binds to the terminal amino acid (TAA) or the modified TAA of the peptide.

55. The method of embodiment 54, further comprising, before the analyzing, cleaving the peptide to generate a cleaved peptide, thereby removing the TAA or the modified TAA to expose a new TAA, and, optionally, modifying the new TAA to yield a newly modified TAA.

56. The method of embodiment 55, further comprising: repeating steps (a), (b), and, optionally, the cleaving one or more times to generate an even further extended nucleic acid recording tag joined to the support, by contacting the cleaved peptide with a third or higher order composition comprising a third or higher order conjugate and a third or higher order nucleic acid moiety, wherein the third or higher order conjugate comprises a third or higher order binding agent that binds to a terminal amino acid (TAA) or a modified TAA of the cleaved peptide or of a derivative of the peptide formed after two or more sequential terminal amino acid cleavages, wherein the third or higher order binding agent is conjugated to a writer enzyme that catalyzes covalent addition of the third or higher order nucleic acid moiety to a terminus of the nucleic acid recording tag extended after previous binding events to generate an even further extended nucleic acid recording tag joined to the support; and by analyzing the even further extended nucleic acid recording tag instead of the further extended nucleic acid recording tag to obtain information regarding binding kinetics and/or selectivity of the first binding agent binding to the peptide; information regarding binding kinetics and/or selectivity of the second binding agent binding to the peptide, and information regarding binding kinetics and/or selectivity of the third or higher order binding agent binding to the peptide, thereby analyzing the peptide.

57. The method of embodiment 55, wherein the first and second compositions are Cycle 1 first and second compositions, respectively, and the Cycle 1 first binding agent binds to the modified NTAA residue of the peptide in a) and the Cycle 1 second binding agent binds to the modified NTAA residue of the peptide in b), wherein the information obtained from Cycle 1 is used to identify the NTAA residue of the peptide, and wherein the method comprises: contacting the peptide with a Cycle 2 first composition comprising a Cycle 2 first conjugate and a Cycle 2 first nucleic acid moiety, wherein the Cycle 2 first conjugate comprises a Cycle 2 first binding agent that binds to the newly exposed NTAA residue or modified newly exposed NTAA residue, wherein the Cycle 2 first binding agent is conjugated to a Cycle 2 first writer enzyme that catalyzes covalent addition of the Cycle 2 first nucleic acid moiety to a terminus of the nucleic acid recording tag after extension in Cycle 1; contacting the peptide with a Cycle 2 second composition comprising a Cycle 2 second conjugate and a Cycle 2 second nucleic acid moiety, wherein the Cycle 2 second conjugate comprises a Cycle 2 second binding agent that binds to the newly exposed NTAA residue or modified newly exposed NTAA residue, wherein the Cycle 2 second binding agent is conjugated to a Cycle 2 second writer enzyme capable of catalyzing covalent addition of the Cycle 2 second nucleic acid moiety to a terminus of the nucleic acid recording tag after extension using the Cycle 2 first nucleic acid moiety; and analyzing the nucleic acid recording tag after extension using the Cycle 2 second nucleic acid moiety to obtain: i) information regarding binding kinetics and/or selectivity of the Cycle 2 first binding agent binding to the newly exposed NTAA residue or a modified newly exposed NTAA residue and ii) information regarding binding kinetics and/or selectivity of the Cycle 2 second binding agent binding to the newly exposed NTAA residue or modified newly exposed NTAA residue, thereby identifying the newly exposed NTAA residue.

58. The method of any one of embodiments 42-57, wherein the Cycle 1 first writer enzyme, the Cycle 1 second writer enzyme, the Cycle 2 first writer enzyme, and the Cycle 2 second writer enzyme each is independently a template-independent polymerase, a DNA ligase, or a RNA ligase.

59. The method of any one of embodiments 42-58, wherein the Cycle 1 first nucleic acid moiety, the Cycle 1 second nucleic acid moiety, the Cycle 2 first nucleic acid moiety, and the Cycle 2 second nucleic acid moiety each is independently a nucleic acid moiety.

60. The method of any one of embodiments 42-59, wherein the Cycle 1 first nucleic acid moiety, the Cycle 1 second nucleic acid moiety, the Cycle 2 first nucleic acid moiety, and the Cycle 2 second nucleic acid moiety each is independently an NTP or a dNTP.

61. The method of any one of embodiments 42-60, wherein the Cycle 1 first nucleic acid moiety, the Cycle 1 second nucleic acid moiety, the Cycle 2 first nucleic acid moiety, and the Cycle 2 second nucleic acid moiety each is independently dCTP, dTTP/dUTP, dGTP, or ATP.

62. The method of any one of embodiments 58-61, wherein the template-independent polymerase is a Terminal deoxynucleotidyl Transferase (TdT).

63. The method of any one of embodiments 42-62, wherein the first binding agent and/or the second binding agent is conjugated to the first or second writer enzyme, respectively, via a first linker.

64. The method of embodiment 63, wherein the first linker is selected from the group consisting of: a peptide, poly alkyl chain (CH₂)ₙ polymer, a poly(ethylene glycol) PEG polymer, a poly(ethylene oxide) PEO polymer, and a combination thereof.

65. The method of any one of embodiments 42-64, wherein the first composition and/or the second composition comprise the first nucleic acid moiety and/or the second nucleic acid moiety covalently tethered to the first or second writer enzyme, respectively, via a second linker.

66. The method of embodiment 65, wherein the second linker comprises a selectively cleavable linkage.

67. The method of any one of embodiments 42-66, wherein during the analyzing step, an artificial intelligence (AI) model, e.g., an AI model employing probabilistic neural networks (PNN), is applied to calculate probabilities of occurrence of specific types of amino acid residues in corresponding positions in amino acid sequence of the peptide based on a nucleotide sequence of the further extended nucleic acid recording tag.

68. The method of any one of embodiments 42-67, wherein the first and/or second writer enzyme catalyzes covalent addition of the first nucleic acid moiety and/or the second nucleic acid moiety to the 3′ hydroxyl of the nucleic acid recording tag or the extended nucleic acid recording tag, respectively.

69. The method of any one of embodiments 42-68, wherein the covalent addition of the first nucleic acid moiety and/or the second nucleic acid moiety occurs for a controlled amount of time.

70. The method of embodiment 69, wherein the controlled amount of time is achieved by using an apyrase-mediated nucleoside degradation.

71. The method of any one of embodiments 42-70, wherein the analyzing comprises using a nucleic acid sequencing method.

72. A method for analyzing a peptide, wherein the peptide and an associated nucleic acid recording tag are joined to a support, the method comprising the steps of:
a) contacting the peptide with a mixture of compositions comprising a first composition and a second composition, wherein (i) the first composition comprises a first conjugate and a first nucleic acid moiety; the first conjugate comprises a first binding agent that binds to the peptide; the first binding agent is conjugated to a first writer enzyme that catalyzes covalent addition of the first nucleic acid moiety to a terminus of the nucleic acid recording tag; and the first nucleic acid moiety is tethered to and controllably cleavable from the first writer enzyme; (ii) the second composition comprises a second conjugate and a second nucleic acid moiety; the second conjugate comprises a second binding agent that binds to the peptide; the second binding agent is conjugated to a second writer enzyme that catalyzes covalent addition of the second nucleic acid moiety to the terminus of the nucleic acid recording tag; and the second nucleic acid moiety is tethered to and controllably cleavable from the second writer enzyme, thereby generating an extended nucleic acid recording tag joined to the support, wherein the extended nucleic acid recording tag comprises covalent addition of the first and/or second nucleic acid moiety;
b) cleaving the first nucleic acid moiety from the first writer enzyme and/or cleaving the second nucleic acid moiety from the second writer enzyme, thereby releasing the first and/or second writer enzyme from the extended nucleic acid recording tag;
c) optionally, repeating steps (a) and (b) one or more times to generate a further extended nucleic acid recording tag joined to the solid support; and
d) analyzing the extended nucleic acid recording tag or the further extended nucleic acid recording tag and obtaining information regarding binding kinetics and/or selectivity of the binding agents bound to the peptide, thereby analyzing the peptide.

73. The method of embodiment 72, wherein the first binding agent binds to a terminal amino acid (TAA) or a modified TAA of the peptide, and the second binding agent binds to the terminal amino acid (TAA) or the modified TAA of the peptide.

74. The method of embodiment 73, further comprising, after step (b) and before step (c), cleaving the peptide to generate a cleaved peptide, thereby removing the TAA or the modified TAA to expose a new TAA, and, optionally, modifying the new TAA to yield a newly modified TAA.

75. The method of any one of embodiments 72-74, wherein the analyzing step comprises a nucleic acid sequencing method.

76. The method of any one of embodiments 72-75, wherein during the analyzing step, an artificial intelligence (AI) model, e.g., an AI model employing probabilistic neural networks (PNN), is applied to calculate probabilities of occurrence of specific types of amino acid residues in corresponding places in amino acid sequence of the peptide based on a nucleotide sequences of the extended and/or the further extended nucleic acid recording tag(s).

77. The method of any one of embodiments 72-76, wherein the writer enzyme catalyzes covalent addition of a nucleic acid moiety to the 3′ hydroxyl of the nucleic acid recording tag.

78. The method of any one of embodiments 72-77, wherein the covalent addition of a nucleic acid moiety to the terminus of the nucleic acid recording tag occurs for a controlled amount of time.

79. The method of embodiment 78, wherein the controlled amount of time is achieved by using an apyrase-mediated nucleoside degradation.

80. The method of embodiment 73, further comprising, before the contacting step (b), modifying an N-terminal amino acid (NTAA) residue of the peptide with a modifying reagent, thereby generating a modified NTAA residue of the peptide.

81. The method of embodiment 73, wherein the first binding agent and the second binding agent are each independently capable of specific binding to a particular type of the modified NTAA residue of the peptide.

82. The method of any one of embodiments 72-81, wherein the writer enzyme is a template-independent polymerase, a DNA ligase, or a RNA ligase.

83. The method of embodiment 82, wherein the template-independent polymerase is a Terminal deoxynucleotidyl Transferase (TdT).

84. A conjugate, which comprises a binding agent conjugated via a first linker to a writer enzyme, wherein said conjugate is configured to bind to a peptide, wherein the peptide and an associated nucleic acid recording tag are joined to a support, said binding agent is configured to bind to said peptide, and said writer enzyme is configured to catalyze covalent addition of a nucleic acid moiety onto a terminus of said nucleic acid recording tag.

85. The conjugate of embodiment 84, which further comprises a nucleic acid moiety that is covalently tethered to the writer enzyme via a second linker comprising a selectively cleavable linkage.

86. The conjugate of embodiment 85, wherein the second linker comprises alkyl, PEG, or PEO moiety with 2-18 chain lengths.

87. The conjugate of embodiment 85, wherein the binding agent is configured to bind to a terminal amino acid (TAA) or a modified TAA of the peptide.

88. A composition comprising a two or more of the conjugates, wherein each conjugate comprises a binding agent conjugated via a first linker to a writer enzyme, wherein each binding agent is configured to bind to a peptide, wherein the peptide and an associated nucleic acid recording tag are joined to a support, and each writer enzyme is configured to catalyze covalent addition of a nucleic acid moiety onto a terminus of each nucleic acid recording tag.

89. The composition of embodiment 88, wherein each binding agent within the two or more of the conjugates is configured to bind to a terminal amino acid (TAA) or a modified TAA of the peptide.

90. The composition of embodiment 89, wherein each binding agent within the two or more of the conjugates has a different selectivity towards terminal amino acids or modified terminal amino acids of peptides.

91. The composition of embodiment 88, wherein each writer enzyme within the two or more of the conjugates is essentially the same.

92. A kit for analyzing or identifying a peptide, which kit comprises a conjugate of any of embodiments 84-87, or a composition of any of embodiments 88-91, and an instruction for using the conjugate or the composition for analyzing or identifying the peptide.

93. The kit of embodiment 92, further comprising a reagent for cleaving the terminal amino acid (TAA) or a modified TAA of the peptide.

### Examples

The following examples are offered to illustrate but not to limit the methods, compositions, and uses provided herein. Certain aspects of the present invention, including, but not limited to, embodiments for the Proteocode™ peptide sequencing assay, information transfer between coding tags and recording tags, methods of making nucleotide-peptide conjugates, methods for attachment of nucleotide-peptide conjugates to a support, methods of generating barcodes, methods of generating specific binders recognizing an N-terminal amino acid of a peptide, reagents and methods for modifying and/or removing an N-terminal amino acid from a peptide, methods for analyzing extended recording tags were disclosed in the following published patent applications: US 2019/0145982 A1, US 2020/0348308 A1, US 2020/0348307 A1, US 2021/0208150 A1, US 2022/0049246 A1, the contents of which are incorporated herein by reference in its entirety.

### Example 1. Immobilization of Recording Tag-Labeled Peptides to a Solid Support

Recording tag-labeled peptides are immobilized on a substrate via an IEDDA click chemistry reaction using an mTet group on the recording tag and a TCO group on the surface of activated beads (solid support). 200 ng of M-270 TCO beads are resuspended in 100 ul phosphate coupling buffer. 5 pmol of DNA recording tag labeled peptides comprising an mTet moiety on the recording tag is added to the beads for a final concentration of 50 nM. The reaction is incubated for 1 hr at room temperature. After immobilization, unreacted TCO groups on the substrate are quenched with 1 mM methyl tetrazine acid in phosphate coupling buffer for 1 hr at room temperature.

Magnetic beads suitable for click-chemistry immobilization are created by converting M-270 amine magnetic Dynabeads to either azide or TCO-derivatized beads capable of coupling to alkyne or methyl Tetrazine-labeled oligo-peptide conjugates, respectively (see also Examples 20-21 of US 2019/0145982 A1). Namely, 10 mg of M-270 beads are washed and resuspended in 500 ul borate buffer (100 mM sodium borate, pH 8.5). A mixture of TCO-PEG (12-120)-NHS (Nanocs) and methyl-PEG (12-120)-NHS is resuspended at 1 mM in DMSO and incubated with M-270 amine beads at room temperature overnight. The ratio of the Methyl to TCO PEG is titrated to adjust the final TCO surface density on the beads such that there is <100 TCO moieties/um2. Unreacted amine groups are capped with a mixture of 0.1M acetic anhydride and 0.1M DIEA in DMF (500 ul for 10 mg of beads) at room temperature for 2 hrs. After capping and washing 3× in DMF, the beads are resuspended in phosphate coupling buffer at 10 mg/ml.

### Example 2. Peptide Immobilization Using Nucleic Acid Hybridization and Joining to a Solid Support

This example describes exemplary methods for joining (immobilizing) nucleic acid-peptide conjugates, such as conjugates of a peptide with recording tag, to a solid support. In a hybridization based method of immobilization, nucleic acid-peptide conjugates were hybridized and ligated to hairpin capture DNAs that were chemically immobilized on magnetic beads. The capture nucleic acids were conjugated to the beads using trans-cyclooctene (TCO) and methyltetrazine (mTet)-based click chemistry. TCO-modified short hairpin capture nucleic acids (16 basepair stem, 5 base loop, 24 base 5′ overhang) were reacted with mTet-coated magnetic beads. Phosphorylated nucleic acid-peptide conjugates (10 nM) were annealed to the hairpin DNAs attached to beads in 5×SSC, 0.02% SDS, and incubated for 30 minutes at 37° C. The beads were washed once with PBST and resuspended in 1× Quick ligation solution (New England Biolabs, USA) with T4 DNA ligase. After a 30-minute incubation at 25° C., the beads were washed twice with PBST and resuspended in the 50 μL of PBST. The total immobilized nucleic acid-peptide conjugates including amino FA-terminal peptides (FAGVAMPGAEDDVVGSGSK; SEQ ID NO: 3), amino AFA-terminal peptides (AFAGVAMPGAEDDVVGSGSK; SEQ ID NO: 4), and an amino AA-terminal peptides (AAGVAMPGAEDDVVGSGSK; SEQ ID NO: 5) were quantified by qPCR using specific primer sets. For comparison, peptides were immobilized onto beads using a non-hybridization based method that did not involve a ligation step. The non-hybridization based method was performed by incubating 30 μM TCO-modified DNA-tagged peptides including amino FA-terminal peptides, amino AFA-terminal peptides, and amino AA-terminal peptides, with mTet-coated magnetic beads overnight at 25° C.

As shown in Table 1, similar Ct values were observed in the non-hybridization preparation method with 1:100,000 grafting density and the hybridization based preparation method with 1:10,000 grafting density. Loading amount of DNA-tagged peptides for the hybridization based preparation method was 1/3000 compared to that for the non-hybridization preparation method. In general, it was observed that less starting material was needed for the hybridization based immobilization method.

TABLE 1
Comparison of Loading Hybridization and Non-
hybridization Immobilization Methods

| | Non-hybridization based | Hybridization based |
|---|---|---|
| Grafting: | immobilization method | immobilization method |
| Passivation | (−Ligation) | (+Ligation) |
| 1:100,000 | 19.4 | 25.4 |
| 1:10,000 | — | 21.1 |

### Example 3. Engineered Dipeptide Cleavases can Remove Single Labeled NTAAs of a Model Peptide

A set of dipeptide cleavase enzymes was evolved from an S46 DPP library as described in the patent application US 2021/0214701 A1, incorporated herein, to recognize and cleave a modified NTAA using M15-L-P1 target peptides (peptide sequences: M15-L-P1-AR, where M15 is an N-terminal peptide modification (2-aminobenzamide), P1 is one of the 17 natural amino acid residues, excluding C, K, R) and the dipeptide aminopeptidase scaffold is from Thermomonas hydrothermalis (SEQ ID NO: 7). The enzymes can efficiently cleave M15-L-labeled peptides between P1 and P2 amino acid residues (the P2 residue is alanine), thus are configured to remove a single labeled terminal amino acid from the peptide (see US 2021/0214701 A1). To accommodate the M15-L label in the substrate binding site, all modified dipeptide cleavases contained the following mutations at the conserved residues that form an amine binding site in unmodified dipeptidyl aminopeptidases: N214M, W215G, R219T, N329R, D673A (the indicated residue numbers correspond to positions of SEQ ID NO: 7). The cleavage efficiency of the evolved enzymes depended on the nature of the P1 residue.

Each evolved cleavase was individually assayed on all M15-L-P1 target peptides. It was found that one selected modified cleavase clone provided 100% cleavage for peptides with the following M15-L-labeled P1 residues: A, I, L, M, Q, V. Other selected modified cleavase clones provided 80-100% cleavage for peptides with the following groups of M15-L-labeled P1 residues: D, E; S,T; G; N; H,Y; F,W. A broad cleavage of a single labeled terminal amino acid from the peptide can be achieved by combining two or more dipeptide cleavases in a set. For example, a set of 7 selected dipeptide cleavases can provide broad activity for removal of almost all M15-L-labeled P1 residues from the peptide (see US 2021/0214701 A1). Other cleavase combinations can be created to achieve a desired level of cleavage specificity, such as different sets of two, three, four or more enzymes.

### Example 4. Peptide Sample Preparation Workflow for the Encoding Assay (See Also US 2022/0144885, Incorporated Herein by Reference in its Entirety for all Purposes)

This example demonstrates an exemplary sample preparation workflow used for preparing peptide-recording tag conjugates and immobilizing them on a solid support.

Protein denaturation and digestion. For a 10 μg of protein sample, samples were diluted to the desired protein input concentration in an appropriate buffer (10 ug/45 μL; 100 mM carbonate/bicarbonate buffer at pH 9.15 with 0.1% sodium dodecyl sulfate (SDS)). Cysteines were reduced with TCEP added to a final concentration of 5 mM. Samples were incubated for 15 min at 37° C., and, after cooling, iodoacetamide (IAA) stock was added to a final concentration of 20 mM. Samples were incubated at 37° C. for 15 min to allow the alkylation to proceed. Lysine side chains were blocked by addition of NHS-acetate (ARRI, 10 mM) at 60° C. for 30 min. Trypsin was added at a 1:25 ratio, by mass, for each sample and incubated for 2 hours at 37° C. to digest the sample. Resulting peptides were then functionalized at the amine terminus using 10 mM photocleavable linker (AAR2, a self-immolative linker comprising para-nitrophenyl carbonate reactive ester coupled to a para-nitrobenzylcarbonate and an PEG-mTET enrichment tag) at 37° C. for 60 min.

Peptide immobilization to solid support. Peptides were immobilized to a solid support (TCO agarose, Click Chemistry Tools) through the enrichment tag (mTET moiety). The peptide mixture was incubated with 130 μL TCO beads for 60 min at 37° C. to immobilize the modified peptides. Other combinations of enrichment tag and compatible solid support can be implemented. Excess material (i.e. cellular components), unreacted peptides, and reaction components were removed by washing three times with PBS-T (PBS (phosphate-buffered saline) plus 0.1% TWEEN® 20).

CHD functionalization of C-terminal arginines and peptide-DNA conjugate formation. Each sample was resuspended after concentration in vacuo in 20 μL 0.2 M NaOH (pH 13.7), 1 M KPhos (pH 8.3), or 2 M KPhos (pH 8.3). CHD Stock (CHD-PEG₃-azide in DMSO) was added for a final concentration of 10 mM and incubated at 37° C. for 1 hr, 80° C. for 1.5 hours, or 80° C. for 1 hour, respectively. The reaction was neutralized by adding equal volume 1 M Tris, pH 7.4, and washed to remove excess/unreacted CHD-PEG₃-azide and impurities. Samples were diluted to 10 μg/1000 μL in PBS-T. On-bead DNA-peptide conjugate (peptide—conjugation reagent—nucleic acid conjugate) formation was carried out using a solution of DBCO-DNA (Dibenzocyclooctyne-coupled DNA; DNA=5′-/5Phos/CAA GTT CTC AGT AAT GCG TAG/DBCOdT/CC GCG ACA CTA G-3′; SEQ ID NO: 9) and incubating for 16 hours. The beads containing the conjugated product were washed to remove excess DBCO-DNA.

Further processing of peptide-DNA conjugates. Upon completion of incubation, beads were centrifuged and washed to remove any excess DBCO-DNA. Sample barcodes were added and beads were washed twice with 200 μL PBS-T. The peptide-DNA chimera was eluted with 10 μL 4 mM biotin, 20 mM Tris-HCl, and 50 mM NaCl. Chimera formation and barcoding were confirmed by loading 0.5 μL of sample (5 pmol) on TBU gel electrophoresis. (15% TBU gel, 200V, 50 min). The peptides were then immobilized on a solid support. The DNA of the peptide-DNA chimera was hybridized and ligated to a DNA recording tag containing a complementary sequence attached to beads at appropriate spacing and density (see Example 3; U.S. application Ser. No. 17/458,199 and WO 2020/223000 A1).

Example 5. Encoding with binding agent fused to a TdT enzyme associated with a gamma-phosphate tethered nucleotide. A fusion protein comprised of an engineered anticalin binding agent fused to a TdT-SpyCatcher construct. Gamma phosphate nucleotides were linked to SpyTag using a maleimide-PEG-NHS crosslinker to conjugate a sulfhydryl group on a terminal cysteine on the SpyTag peptide to the amine on the dG4P-heptyl-NH₂ nucleotide (Kumar, 2012).

### Example 6. Synthesis of 2′-deoxyguanosine-5′-tetraphosphate (dG4P)

The synthesis of 2′-dG4P is carried out starting from 2′-dGTP as disclosed in (Kumar, et al., 2012. “PEG-Labeled Nucleotides and Nanopore Detection for Single Molecule DNA Sequencing by Synthesis.” Scientific Reports 2 (1): 1-8). 300 mmoles of 2′-dGTP (triethylammonium salt) are converted to the tributylammonium salt by using 1.5 mmol (5 eq) of tributylamine in anhydrous pyridine (5 ml). The resulting solution is concentrated to dryness and co-evaporated twice with 5 ml of anhydrous DMF. The dGTP (tributylammonium salt) is dissolved in 5 ml anhydrous DMF, and 1.5 mmol 1, 1-carbonyldiimidazole (CDI) is added. The reaction is stirred for 6 hr, after which 12 ml methanol is added and stirring continued for 30 min. To this solution, 1.5 mmol phosphoric acid (tributylammonium salt, in DMF) is added and the reaction mixture is stirred overnight at room temperature. The reaction mixture is diluted with water and purified on a Sephadex-A25 column using a 0.1 M to 1 M TEAB gradient (pH 7.5). The dG4P elutes at the end of the gradient. The appropriate fractions are combined and further purified by reverse-phase HPLC to yield 175 mmol of the pure tetraphosphate (dG4P).

### Example 7. Synthesis of dG4P-heptyl-NH₂

Using a protocol adapted from Kumar, et al. 2012, to 80 mmol dG4P in 2 ml water and 3.5 ml 0.1 M 1-methylimidazole-HCl (pH 6) are added 154 mg EDAC and 260 mg diaminoheptane. The pH of the resulting solution is adjusted to 6 with concentrated HCl and stirred at room temperature overnight. This solution is diluted with water and purified by Sephadex-A25 ion-exchange chromatography followed by reverse phase HPLC to yield 20 mmol dG4P-heptyl-NH₂.

Alternate Protocols for synthesis of dG4P-heptyl-NH₂ are disclosed in U.S. Pat. No. 10,443,096 B2; Shepard, et al., 2019. “Nucleoside Tetra- and Pentaphosphates Prepared Using a Tetraphosphorylation Reagent Are Potent Inhibitors of Ribonuclease A.” Journal of the American Chemical Society 141 (46): 18400-404; and Mohamady and Taylor. 2013. “Synthesis of Nucleoside Tetraphosphates and Dinucleoside Pentaphosphates via Activation of Cyclic Trimetaphosphate.” Organic Letters 15 (11): 2612-15.

### Example 8. Synthesis of Maleimide-PEGₙ-dG4P

Gamma phosphate nucleotides are linked to SpyTag using a maleimide-PEG-NHS crosslinker to conjugate a sulfhydryl group on a terminal cysteine on the SpyTag peptide to the amine on the dG4P-heptyl-NH₂ nucleotide (Kumar, et al., 2012). Using a protocol adapted from Kumar et al., 2012, dG4P-heptyl-NH₂ synthesized above is taken up in 0.1 M sodium carbonate-bicarbonate buffer (pH 8.6) and to this stirred solution is added 1 eq. of one of the maleimide-PEGₙ-NHS esters (n=4, 6, 8, or 12, BroadPharm) in DMF. The resulting mixture is stirred overnight at room temperature and then purified on a silica-gel cartridge (15-25% MeOH in CH₂Cl₂ to remove unreacted maleimide-PEGₙ-NHS ester and eluted with 5:4:1 isopropanol/NH₄OH/H₂O). The crude product is further purified twice by reverse-phase HPLC to provide pure Maleimide-PEGₙ-dG4P.

### Example 9. Synthesis of SpyCatcher-PEGₙ-dG4P

The SpyCatcher-SpyTag system is one of the most efficient labeling systems available (see, e.g., Reddington and Howarth, M. (2015). Secrets of a covalent interaction for biomaterials and biotechnology: SpyTag and SpyCatcher. Curr Opin Chem Biol, 29, 94-99; Zakeri et al., 2012. Peptide tag forming a rapid covalent bond to a protein, through engineering a bacterial adhesin. Proc Natl Acad Sci USA, 109(12), E690-697). SpyCatcher is a compact 116 residue protein that efficiently forms a covalent isopeptide bond (under a broad range of coupling conditions) with SpyCatcher, a 13 amino acid peptide (AHIVMVDAYKPTK, SEQ ID NO: 27). Various engineered variations of the SpyCatcher-SpyTag system have improved on reaction rates, and led to orthogonal coupling systems such as SnoopTag and SnoopCatcher (Hatlem et al., 2019). SpyCatcher can easily be recombinantly expressed with the protein of interest. Moreover, the SpyTag peptide can be pre-conjugated to a primary amine compound via SMCC coupling to an N-terminal cysteine residue on the SpyTag (CAHIVMVDAYKPTK, SEQ ID NO: 28). SpyTag bioconjugation with the maleimide-PEGₙ-dG4) is achieved by mixing 1.0 eq. of N-terminal cysteine SpyTag peptide with 0.5 eq. of maleimide-PEGₙ-dG4P in PBS buffer and incubating for 1 hr at room temperature. The resulting product is purified by silica-gel cartridge (15-25% MeOH in CH₂Cl₂ to remove unreacted SpyTag peptide and then eluted with 5:4:1 isopropanol/NH₄OH/H₂O). The resultant SpyTag-PEGn-dG4P product is aliquoted, lyophilized, and stored at −80° C.

### Example 10. Synthesis of BA-TdT-PEGn-dG4P Complex

The purified BA-TdT-SpyCatcher fusion protein is incubated with 1.5 eq. of SpyTag-PEGn-dG4P reagent in PBS buffer for 1 hr at 37° C. to covalently couple the SpyTag peptide to the fused SpyCatcher protein via a self-formed isopeptide bond. The resultant final product, BA-TdT-PEGn-dG4P, is purified from unreacted SpyTag-PEGn-dG4P reagent using size exclusion chromatography. BA-TdT-PEGn-dA4P, BA-TdT-PEGn-dC4P, and BA-TdT-PEGn-dT4P are synthesized using similar protocols to those described above except starting with 2′-dATP, 2′-dCTP, and 2′-dTTP, respectively.

### Example 11. Kinetic Encoding with a Binding Agent—Writer Enzyme Fusion Comprised of a TdT-Nucleotide-Binding Agent—TdT Fusion Protein Labeled with Polyphosphate Nucleotides

Kinetic encoding with binder-writer fusions is initiated by incubating peptides and an associated nucleic acid recording tag joined to a solid support with binder-writer fusions as described in Example 9 comprised of BA-TdT-PEGn-dN4P complexes (where N can be A, C, G, or T). Kinetic encoding is performed using a pool of binder-writer-nucleotide complexes (˜10-100 nM per binder-writer complex) in a Kinetic Encoding buffer (50 mM potassium acetate, 20 mM Tris-acetate, 10 mM magnesium acetate, pH 7.9 @ 25° C.) supplemented with 0.5 mM CoCl₂ at 37° C. for 1-30 min. Yeast inorganic pyrophosphatase (NEB) was included at 1 mU/ul to prevent pyrophosphorolysis. After completion of kinetic encoding, the ProteoCode substrate is washed with 0.1 N NaOH, and then 1× with high phosphate HPBS buffer and 2× with PBS buffer at 37° C. The ProteoCode substrate is now ready for the next cycle ofN-terminal cleavage, binding and kinetic encoding.

### Example 12. Binder-Writer Configuration Using pdPolθ with Tethered Nucleotides

A binder-writer configuration, BA-pdPolθ-PEGn-dN4P complex (where N can be A, C, G, or T) was generated as described in Examples 6-9, wherein the BA-TdT fusion is replaced with the BA-pdPolθ fusion with a dNTP tethered via the SpyCatcher-SpyTag approach outlined in Example 9. pdPolθ is comprised of the recombinant human Pol (polymerase domain; residues 1792-2590) theta polymerase (SEQ ID NO: 11).

### Example 13. Expression/Purification of pdPol

Adapting a protocol from Hogg et al. (Hogg et al., 2012), pSUMO3 vectors containing the wild type and mutant polymerase genes are transformed into Rosetta2(DE3)/pLysS cells (Stratagene). Colonies are grown up in autoinduction medium (1× Terrific Broth (USB Corporation), 0.5% w/v glycerol, 0.05% w/v dextrose, 0.2% w/v alpha-lactose, 100 ug/ml ampicillin and 34 ¬μg/ml chloramphenicol) shaken at 20° C. for 60 hours. The resulting E. coli pellets are stored at −80° C. Frozen pellets are thawed on ice and resuspended in buffer containing 50 mM HEPES pH 8, 300 mM NaCl, 10% (v/v) glycerol, 20 mM imidazole pH 8, 5 mM CaCl₂), 1.5% (v/v) NP-40 substitute (Fluka), 5 mM 2-mercaptoethanol (BME), 10 mM PMSF, 100 mM benzamidine and 500 mg DNase I at a volume of 5 ml of buffer per gram of cell pellet. The resuspended cells are sonicated on ice then spun down twice at 27,000 g. For His-tag purification, clarified cell lysate is loaded onto a 5 ml His-Trap column (GE Lifesciences) and washed with buffer A (50 mM HEPES pH 8, 300 mM NaCl, 10% (v/v) glycerol, 20 mM imidazole pH 8, 5 mM BME and 0.005% v/v NP-40 substitute). Bound His-tag material is eluted with buffer B (50 mM HEPES pH 8, 300 mM NaCl, 10% (v/v) glycerol, 0.005% (v/v) NP-40 substitute, 5 mM BME and 125 mM imidazole pH 8). Eluted fractions are purified over a type-II ceramic hydroxyapatite (Bio-Rad) column and washed with buffer C (50 mM HEPES pH 8, 300 mM NaCl, 10% (v/v) glycerol, 0.005% NP-40, and 5 mM BME). Bound fractions are eluted with a shallow gradient to 10% buffer D (500 mM K2HPO4/KH2PO4 pH 8, 300 mM NaCl, 10% (v/v) glycerol, 0.005% NP-40, and 5 mM BME).

Eluted fractions are loaded onto a 5 ml Heparin Hi-Trap column (GE Lifesciences) and washed with buffer C. Bound fractions are eluted with a gradient to buffer E (50 mM HEPES pH 8, 2 M NaCl, 10% (v/v) glycerol, 0.005% (v/v) NP-40, and 5 mM BME). Fractions containing POLQ are pooled and incubated with 5 units of SUMO protease 2 (LifeSensors) for 2 hours. The digested fractions are then loaded onto a 5 ml His-Trap column and washed with buffer C. Cleaved POLQ is separated from uncleaved POLQ and the protease by applying a gradient to buffer B. POLQ fractions are concentrated to 0.5 ml and run through a 25 ml Superdex GS-200 column (GE Lifesciences) pre-equilibrated with buffer C. Fractions containing POLQ are concentrated, frozen in 5 μl aliquots by plunging into liquid nitrogen and stored at −80° C. All steps in the purification process are carried out at 4° C.

### Example 14. Binder-Writer Configuration Using Three Tethered Nucleotides to Generate Triplet Codons for Encoding

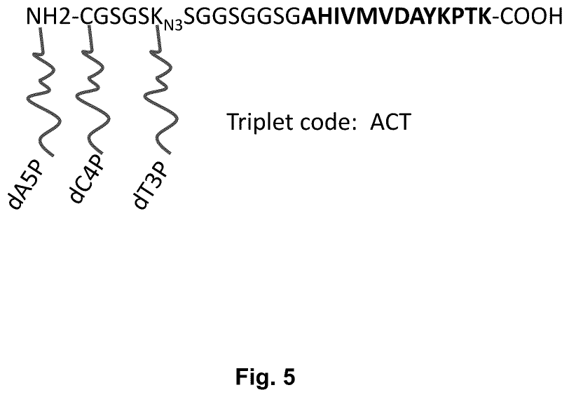

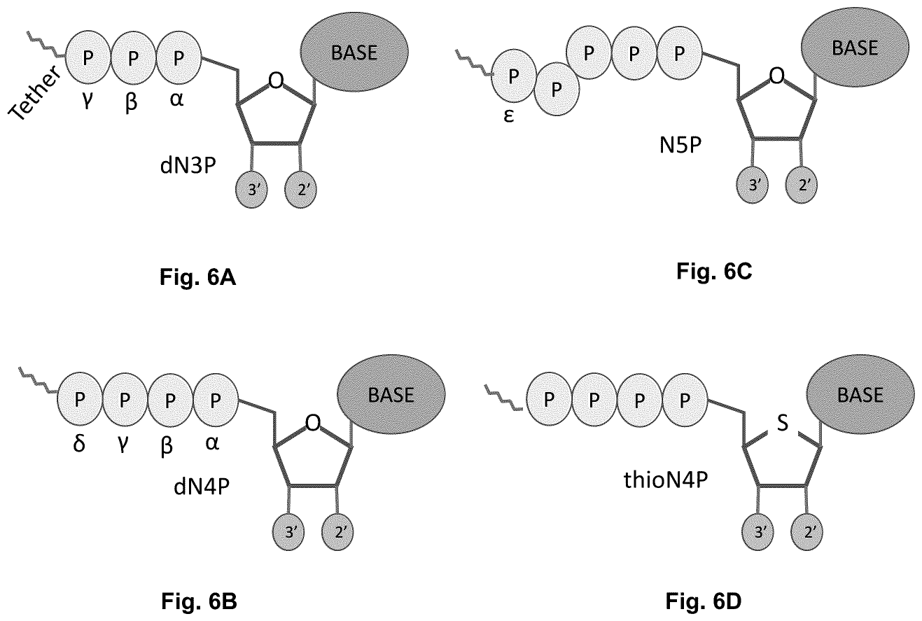

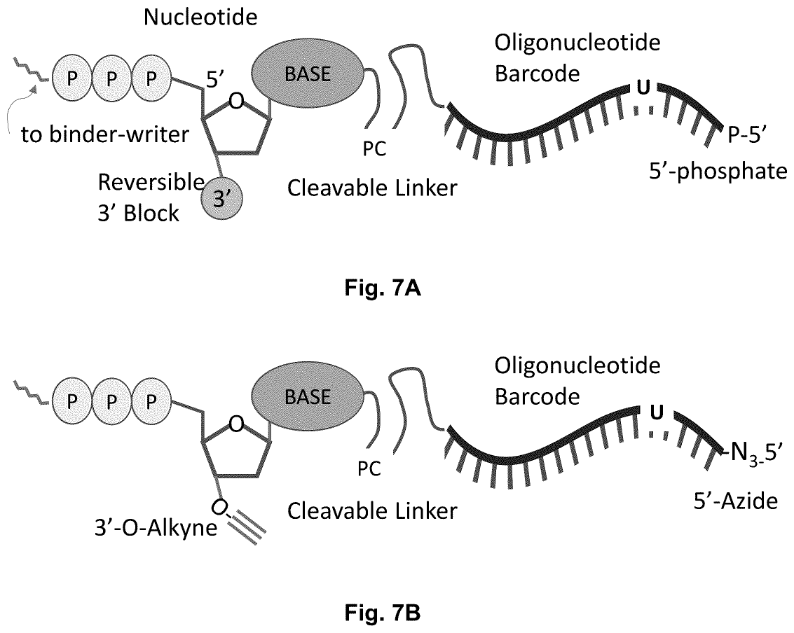

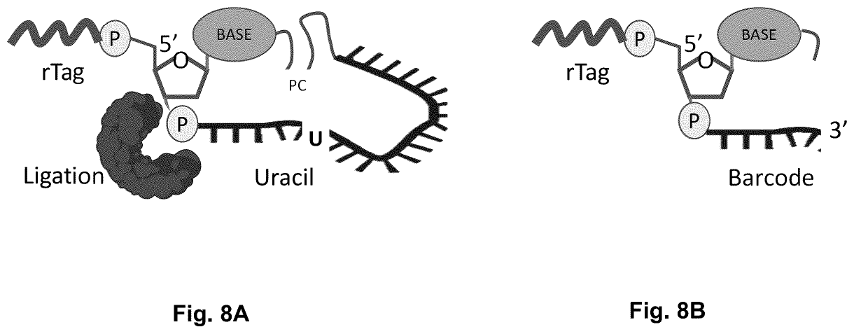

Using the SpyTag approach, three tethered nucleotides can be added to a SpyTag peptide by including an N-terminal cysteine group that can be dual labeled by first coupling a thioester-derivatized nucleotide (e.g., thioester-PEGn-dA5P) to the 1,2-aminothiol exposed by the N-terminal cysteine group followed by maleimide coupling of a maleimide-derivatized nucleotide (e.g., maleimide-PEGn-dC4P) to the resulting thiol of the cysteine group (De Rosa, et al., 2021. “Exploiting Protein N-Terminus for Site-Specific Bioconjugation.” Molecules 26 (12)). The coupling of the third nucleotide can be accomplished by reacting a lysine azide with an alkyl-derivatized nucleotide (alkyl-PEGn-dTTP) using CuAAC click chemistry (see FIG. 5). Both thioester nucleotides and alkyl nucleotides can be generated from NHS derivitized nucleotides by treatment with thiols, and propargylamines, respectively. Alternatively, alkyl-labeled phosphate nucleotides can be synthesized as described by Serdjukow et al. (Serdjukow, et al., 2014. “Synthesis of γ-Labeled Nucleoside 5′-Triphosphates Using Click Chemistry.” Chemical Communications 50 (15): 1861-63). Peptide linkers (e.g. GGGS, GSGS, GSGTAGGGSGS, SGGSGGSG, see SEQ ID NO: 30-33) can be used between the N-terminal cysteine and azide lysine and between the azide lysine and the SpyTag to provide better accessibility to labeling and downstream SpyCatcher-SpyTag bioconjugation. When this trinucleotide-conjugated SpyTag is coupled to the binder-writer SpyCatcher fusion protein, the binder-writer construct is capable of writing three nucleotides to a proximal recording tag, in this case A, C, and T nucleotides in sequential order due to differing rates of coupling arising from the tri vs tetra vs penta-phosphate linkages to the nucleotides (Sood, et al, 2005).

### Example 15. Evaluating Efficiency of Nucleic Acid Moiety Incorporation into Recording Tag During Encoding Process Using TdT-F4R10 Conjugate

Feasibility of the encoding approach disclosed above has been tested experimentally using model TdT-F4R10 conjugate (the sequence of the conjugate is set forth in SEQ ID NO: 16). F4R10 is a binder that specifically binds to F NTAA residue of immobilized peptides (F-binder), with lesser specificity towards Y and W NTAA residues. Both F-binder and methods for immobilization of peptides (attachment to a solid support, such as beads) are disclosed in US 2022/0049246 A1, incorporated herein. The recombinant gene encoding the amino acid sequence set forth in SEQ ID NO: 16 was synthesized and cloned into pET-28b vector and overexpressed in E. coli BL21(DE3) strain. The conjugate protein was purified from the soluble fraction of bacterial lysates using tandem immobilized metal affinity chromatography and size exclusion chromatography.

To evaluate specificity of encoding reactions, the encoding reactions were performed on an immobilized set of 484 peptides (22×22 combination of different P1 and P2 residues, see FIG. 10), wherein each peptide is associated with a DNA recording tag, to generate a heatmap array where each cell of the array represents an encoding efficiency of the given binder that binds to a specific combination of P1-P2 residues of the target peptide. Similar peptide array was disclosed in the U.S. patent application Ser. No. 17/539,033. The encoding efficiency was calculated as TIE_len=average number of nucleotide bases added after 4 cycles of encoding using four different dNTPs, which were consecutive added to the reactions (FIG. 10). A Terminal transferase from New England Biolabs (NEB) was used as a control. Encoding reactions with the TdT-F4R10 conjugate or free TdT (NEB) were performed in the TdT buffer (NEB). A solution of 50 ul of F4R10-TdT fusion (100 nM) or TdT (0.5 unit/uL) with an individual dNTP (300 nM) in TdT buffer ((50 mM Potassium Acetate, 20 mM Tris-acetate, 10 mM Magnesium Acetate, 250 uM CoCl2, pH 7.9, NEB) were added to 2,000 beads containing an immobilized peptide in a well of 96-well filter plate, and each individual dNTP was added subsequently in the following order: dCTP->dGTP->dTTP->dATP. Washing was done to remove an individual dNTP used during each encoding reaction, before new individual dNTP was added. Each encoding reaction (incorporation of individual dNTP to the terminus of the recording tag) was continued at 37° C. for 5 minutes, after which beads were washed twice with PBST buffer (150 uL).

To calculate average number of nucleotide bases added after 4 cycles of encoding, extended recording tags associated with peptides were amplified and subjected to nucleic acid sequencing (NGS). Ligation of the Illumina sequencing adapter (5′ pre-adenylated 3′-blocked DNA; the sequence is set forth in SEQ ID NO: 29) was carried out with 1 uM of Thermostable 5′ App DNA/RNA Ligase (NEB, cat. no. M0319L) at 60° C. overnight in ligation buffer containing 10 mM Bis-Tris-Propane-HCl, 10 mM MgCl2, 50 mM MnCl2, 1 mM DTT.

FIG. 10 shows encoding heatmaps obtained by using a free TdT enzyme (left panel) and TdT-F4R10 conjugate (right panel). TIE_len is an average number of nucleotide bases incorporated into corresponding recording tags after 4 cycles of encoding using four individual dNTPs as described above. Left panel shows no specificity for number of nucleotide bases incorporated into corresponding recording tags (incorporation occurs unspecifically because of a high TdT concentration used). In contrast, the right panel of FIG. 10 shows preferential encoding by the TdT-F4R10 conjugate across peptides having F, Y and W NTAA residues, consistent with the known binding selectivity of the F4R10 binder. At the tested concentration, the TdT-F4R10 conjugate inserts on average about 5-8 nt per encoding cycle for most F-P2 peptides. The number of incorporated nucleotide bases can be reduced by changing the concentration of components in the reaction. Thus, binding selectivity of the binder present in the binder-writer conjugate translates into specificity for the writer enzyme, which incorporates nucleic acid moieties (e.g., nucleotide moieties) into terminus of the recording tag, allowing peptide's structural information and binder-peptide kinetic information to be encoded into nucleotide sequence of the recording tag.

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for analyzing a peptide, wherein the peptide and an associated nucleic acid recording tag are joined to a support, the method comprising:
a) contacting the peptide with a first composition comprising a first conjugate and a first nucleic acid moiety, wherein the first conjugate comprises a first binding agent that binds to the peptide, wherein the first binding agent is conjugated to a first writer enzyme that catalyzes covalent addition of the first nucleic acid moiety to a terminus of the nucleic acid recording tag to generate an extended nucleic acid recording tag joined to the support;
b) contacting the peptide with a second composition comprising a second conjugate and a second nucleic acid moiety, wherein the second conjugate comprises a second binding agent that binds to the peptide, wherein the second binding agent is conjugated to a second writer enzyme that catalyzes covalent addition of the second nucleic acid moiety to a terminus of the extended nucleic acid recording tag to generate a further extended nucleic acid recording tag joined to the support; and
c) analyzing the further extended nucleic acid recording tag to obtain information regarding binding kinetics and/or selectivity of the first binding agent binding to the peptide and information regarding binding kinetics and/or selectivity of the second binding agent binding to the peptide, thereby analyzing the peptide.

2. The method of claim 1, further comprising:
contacting the peptide with a third composition comprising a third conjugate and a third nucleic acid moiety, wherein the third conjugate comprises a third binding agent that binds to the peptide, wherein the third binding agent is conjugated to a third writer enzyme that catalyzes covalent addition of the third nucleic acid moiety to a terminus of the further extended nucleic acid recording tag to generate an even further extended nucleic acid recording tag joined to the support, and
analyzing the even further extended nucleic acid recording tag to obtain information regarding binding kinetics and/or selectivity of the third binding agent binding to the peptide.

3. The method of claim 1, wherein the contacting in (a) and the contacting in (b) are performed in a sequential order.

4. The method of claim 1, wherein the contacting in (a) and the contacting in (b) are performed at the same time.

5. The method of claim 1, wherein a plurality of peptides are contacted with the first composition in (a) and with the second composition in (b), and wherein each peptide from the plurality of peptides is independently associated with a nucleic acid recording tag, and each peptide and the associated nucleic acid recording tag is joined to the support.

6. The method of claim 1, further comprising, before (a), modifying an N-terminal amino acid (NTAA) residue of the peptide with a modifying reagent, thereby generating a modified NTAA residue of the peptide.

7. The method of claim 1, wherein the first binding agent binds to a terminal amino acid (TAA) or a modified TAA of the peptide, and the second binding agent binds to the terminal amino acid (TAA) or the modified TAA of the peptide.

8. The method of claim 7, further comprising, before the analyzing, cleaving the peptide to generate a cleaved peptide, thereby removing the TAA or the modified TAA to expose a new TAA, and, optionally, modifying the new TAA to yield a newly modified TAA.

9. The method of claim 8, further comprising: repeating steps (a), (b), and, optionally, the cleaving one or more times to generate an even further extended nucleic acid recording tag joined to the support, by contacting the cleaved peptide with a third or higher order composition comprising a third or higher order conjugate and a third or higher order nucleic acid moiety, wherein the third or higher order conjugate comprises a third or higher order binding agent that binds to a terminal amino acid (TAA) or a modified TAA of the cleaved peptide or of a derivative of the peptide formed after two or more sequential terminal amino acid cleavages, wherein the third or higher order binding agent is conjugated to a writer enzyme that catalyzes covalent addition of the third or higher order nucleic acid moiety to a terminus of the nucleic acid recording tag extended after previous binding events to generate an even further extended nucleic acid recording tag joined to the support; and by
analyzing the even further extended nucleic acid recording tag instead of the further extended nucleic acid recording tag to obtain: information regarding binding kinetics and/or selectivity of the first binding agent binding to the peptide; information regarding binding kinetics and/or selectivity of the second binding agent binding to the peptide; and information regarding binding kinetics and/or selectivity of the third or higher order binding agent binding to the peptide, thereby analyzing the peptide.

10. The method of claim 8, wherein the first and second compositions are Cycle 1 first and second compositions, respectively, and the Cycle 1 first binding agent binds to the modified NTAA residue of the peptide in a) and the Cycle 1 second binding agent binds to the modified NTAA residue of the peptide in b),
wherein the information obtained from Cycle 1 is used to identify the NTAA residue of the peptide, and
wherein the method comprises:
contacting the peptide with a Cycle 2 first composition comprising a Cycle 2 first conjugate and a Cycle 2 first nucleic acid moiety, wherein the Cycle 2 first conjugate comprises a Cycle 2 first binding agent that binds to the newly exposed NTAA residue or modified newly exposed NTAA residue, wherein the Cycle 2 first binding agent is conjugated to a Cycle 2 first writer enzyme that catalyzes covalent addition of the Cycle 2 first nucleic acid moiety to a terminus of the nucleic acid recording tag after extension in Cycle 1;
contacting the peptide with a Cycle 2 second composition comprising a Cycle 2 second conjugate and a Cycle 2 second nucleic acid moiety, wherein the Cycle 2 second conjugate comprises a Cycle 2 second binding agent that binds to the newly exposed NTAA residue or modified newly exposed NTAA residue, wherein the Cycle 2 second binding agent is conjugated to a Cycle 2 second writer enzyme that catalyzes covalent addition of the Cycle 2 second nucleic acid moiety to a terminus of the nucleic acid recording tag after extension using the Cycle 2 first nucleic acid moiety; and
analyzing the nucleic acid recording tag after extension using the Cycle 2 second nucleic acid moiety to obtain: i) information regarding binding kinetics and/or selectivity of the Cycle 2 first binding agent binding to the newly exposed NTAA residue or a modified newly exposed NTAA residue and ii) information regarding binding kinetics and/or selectivity of the Cycle 2 second binding agent binding to the newly exposed NTAA residue or modified newly exposed NTAA residue, thereby identifying the newly exposed NTAA residue.

11. The method of claim 1, wherein the first writer enzyme and the second writer enzyme each is independently a template-independent polymerase, a DNA ligase, or a RNA ligase.

12. The method of claim 1, wherein the first composition and/or the second composition comprise the first nucleic acid moiety and/or the second nucleic acid moiety covalently tethered to the first or second writer enzyme, respectively, via a second linker.

13. The method of claim 12, wherein the second linker comprises a selectively cleavable linkage.

14. The method of claim 1, wherein during the analyzing step, an artificial intelligence (AI) model is applied to calculate probabilities of occurrence of one or more particular types or classes of amino acid residues in corresponding positions in amino acid sequence of the peptide based on a nucleotide sequence of the further extended nucleic acid recording tag.

15. The method of claim 1, wherein the first and/or second writer enzyme catalyzes covalent addition of the first nucleic acid moiety and/or the second nucleic acid moiety to the 3′ hydroxyl of the nucleic acid recording tag or the extended nucleic acid recording tag, respectively.

16. The method of claim 1, wherein the covalent addition of the first nucleic acid moiety and/or the second nucleic acid moiety occurs for a controlled amount of time.

17. A method for analyzing a peptide, wherein the peptide and an associated nucleic acid recording tag are joined to a support, the method comprising the steps of
a) contacting the peptide with a mixture of compositions comprising a first composition and a second composition, wherein (i) the first composition comprises a first conjugate and a first nucleic acid moiety; the first conjugate comprises a first binding agent that binds to the peptide; the first binding agent is conjugated to a first writer enzyme that catalyzes covalent addition of the first nucleic acid moiety to a terminus of the nucleic acid recording tag; and the first nucleic acid moiety is tethered to and controllably cleavable from the first writer enzyme; (ii) the second composition comprises a second conjugate and a second nucleic acid moiety; the second conjugate comprises a second binding agent that binds to the peptide; the second binding agent is conjugated to a second writer enzyme that catalyzes covalent addition of the second nucleic acid moiety to the terminus of the nucleic acid recording tag; and the second nucleic acid moiety is tethered to and controllably cleavable from the second writer enzyme, thereby generating an extended nucleic acid recording tag joined to the support, wherein the extended nucleic acid recording tag comprises covalent addition of the first and/or second nucleic acid moiety;
b) cleaving the first nucleic acid moiety from the first writer enzyme and/or cleaving the second nucleic acid moiety from the second writer enzyme, thereby releasing the first and/or second writer enzyme from the extended nucleic acid recording tag;
c) optionally, repeating steps (a) and (b) one or more times to generate a further extended nucleic acid recording tag joined to the solid support; and
d) analyzing the extended nucleic acid recording tag or the further extended nucleic acid recording tag and obtaining information regarding binding kinetics and/or selectivity of the binding agents bound to the peptide, thereby analyzing the peptide.

18. The method of claim 17, wherein the first binding agent binds to a terminal amino acid (TAA) or a modified TAA of the peptide, and the second binding agent binds to the terminal amino acid (TAA) or the modified TAA of the peptide.

19. The method of claim 18, further comprising, after step (b) and before step (c), cleaving the peptide to generate a cleaved peptide, thereby removing the TAA or the modified TAA to expose a new TAA, and, optionally, modifying the new TAA to yield a newly modified TAA.

20. The method of claim 17, wherein during the analyzing step, an artificial intelligence (AI) model, e.g., an AI model employing probabilistic neural networks (PNN), is applied to calculate probabilities of occurrence of one or more particular types or classes of amino acid residues in corresponding places in amino acid sequence of the peptide based on a nucleotide sequences of the extended and/or the further extended nucleic acid recording tag(s).

21. The method of claim 17, wherein the writer enzyme catalyzes covalent addition of a nucleic acid moiety to the 3′ hydroxyl of the nucleic acid recording tag.

22. The method of claim 17, wherein the covalent addition of a nucleic acid moiety to the terminus of the nucleic acid recording tag occurs for a controlled amount of time.

23. The method of claim 18, further comprising, before the contacting step (b), modifying an N-terminal amino acid (NTAA) residue of the peptide with a modifying reagent, thereby generating a modified NTAA residue of the peptide.

24. The method of claim 17, wherein the first writer enzyme and the second writer enzyme each is independently a template-independent polymerase, a DNA ligase, or a RNA ligase.

25. A composition comprising two or more conjugates, wherein each conjugate comprises a binding agent conjugated via a first linker to a writer enzyme, wherein each binding agent is configured to bind to a peptide, wherein the peptide and an associated nucleic acid recording tag are joined to a support, and each writer enzyme is i) configured to catalyze covalent addition of a nucleic acid moiety onto a terminus of each nucleic acid recording tag, and ii) a template-independent polymerase, a DNA ligase, or a RNA ligase.

26. The composition of claim 25, wherein each binding agent within the two or more of the conjugates is configured to bind to a terminal amino acid (TAA) or a modified TAA of the peptide.

27. The composition of claim 26, wherein each binding agent within the two or more of the conjugates has a different selectivity towards terminal amino acids or modified terminal amino acids of peptides.

28. The composition of claim 25, wherein each writer enzyme within the two or more of the conjugates is essentially the same.

29. The composition of claim 25, wherein each conjugate further comprises a nucleic acid moiety covalently tethered to the writer enzyme via a second linker.
